(19) United States
(12) Patent Application Publication
(10) Pub. No.: US 2018/0221503 A1
(43) Pub. Date: Aug. 9, 2018

(54) COMPOSITIONS AND METHODS FOR IMMUNO-ONCOLOGY THERAPIES

(71) Applicant: TARVEDA THERAPEUTICS, INC., Watertown, MA (US)

(72) Inventors: Sudhakar Kadiyala, Newton, MA (US); Donna T. Ward, Groton, MA (US); Richard Wooster, Natick, MA (US)

(21) Appl. No.: 15/749,180

(22) PCT Filed: Jul. 29, 2016
(86) PCT No.: PCT/US16/44775

§ 371 Date: Jan. 31, 2018

Related U.S. Application Data
(60) Provisional application No. 62/199,414, filed on Jul. 31, 2015.
(60) Provisional application No. 62/332,772, filed on May 6, 2016.

(51) Int. Cl. A61K 47/64; A61K 47/68; A61K 47/69; A61P 35/00

(52) U.S. Cl. CPC A61K 47/646; A61K 47/642; A61K 47/643; A61P 35/00; A61K 47/6845; A61K 47/6937; A61K 47/644

(57) ABSTRACT

The present invention relates to cancer immunotherapy. Conjugates and nanoparticles comprising active agents that can elicit a cancer specific immune response are provided. The conjugates comprise one or more targeting moieties that are connected to the active agents. Nanoparticles comprising the conjugates of the present invention are also provided to increase the delivery of the conjugates, and increase immunogenicity and lower toxicity.

### REFERENCE TO RELATED APPLICATIONS

The present application claims priority to U.S. Provisional Patent Application No. 62/199,414, filed Jul. 31, 2015, entitled COMPOSITIONS AND METHODS FOR IMMUNO-ONCOLOGY THERAPIES, and U.S. Provisional Patent Application No. 62/332,772, filed May 6, 2016, entitled COMPOSITIONS AND METHODS FOR IMMUNO-ONCOLOGY THERAPIES, the contents of each of which are herein incorporated by reference in their entirety.

### FIELD OF THE INVENTION

The present invention relates to the field of immuno-oncology therapy. In particular, the present invention relates to immune modulating conjugates and particles packaging such conjugates.

### BACKGROUND OF THE INVENTION

Cancer is a heterogeneous disease that results from a multi-step process, characterized by uncontrolled tumor cell proliferation, invasion and metastasis. Tumor cells have also the ability to evade cell death and to escape immune system surveillance (Zitvogel et al., Nat. Rev. Immunol. 2006, 6:715-727). With a more detailed understanding of the interaction between the immune system and cancer, cancer immunotherapy has become a promising therapeutic strategy for treatment of cancer.

The immune system can recognize tumor cells, parts of tumor cells or specific substances isolated from tumor cells and respond to these malignant cells. Both the innate and adaptive immune subsystems can respond to tumor cells in vivo. In the adaptive immune response, antigen presenting cells (e.g., dendritic cells) can capture and present tumor specific antigens to naïve T cells producing activated T-cells. Activated cancer antigen specific T cells can recognize and destroy tumor cells presenting epitopes to which the T cells have been primed. The ability to exploit the immune system has brought new insights into the development of novel cancer immunotherapy treatments.

Approaches that aim to enhance cancer specific immune responses have been largely developed for a variety of cancers. Monoclonal antibodies that are engaged with tumor mechanisms are used clinically such as trastuzumab for breast cancer (Kirkwood et al., CA Cancer J. Clin., 2012, 62: 309-335). Cancer vaccinations directed to strengthen the immune system for the destruction of tumors has shown encouraging preclinical results and has been extensively explored (e.g., Palucka and Banchereau, Nat. Rev. Cancer, 2012, 12: 265-277). The recognition of the critical role of T cells, particularly cytotoxic T lymphocytes (CTLs), in cancer for immune-based treatment has contributed to the extensive research and development of strategies to increase their anti-cancer activity. Among various approaches, adoptive T cell immunotherapy has had impressive success in treating malignant and infection diseases. Autologous T cells are cultured and/or engineered ex vivo and adoptively transferred into the patient. T cells are directly targeted in vivo by vaccination or biological compounds. Regardless of the approach taken, these immunotherapies generate a de novo T cell-mediated immune response and/or enhance preexisting functions, which are often suppressed in patients (Reviewed by Perica et al., Adoptive T cell immunotherapy, 2015, Rambam Maimonides Med J. 6(1): e0004).

Cancer immunotherapies may be suitable for a large number of cancer types. Immuno-oncology therapies are now available to patients with advanced melanoma and prostate cancer (Kantoff et al., N Engl J Med, 2010, 363: 411-422).

The present invention provides novel conjugates and nanoparticles for targeted immunotherapy. The conjugates and nanoparticles described here in can increase the delivery of immunologic agents such as tumor specific antigens, artificial antigen presenting cells, T cell agents that can activate T cells, antibodies, cytokines and other immune stimulating agents to a targeted tissue (e.g., a metastatic site, or a lymph node), and/or a particular cell type of interest such as tumor cells and a type of immune cells. The conjugates and nanoparticles provide flexibility for combining different agents that function for different mechanisms to the same conjugate or the same particle; such combinations may synergistically enhance the efficacy of cancer immunotherapy. In addition, the conjugates and nanoparticles are useful in the sustained release of immunologic active agents.

### SUMMARY OF THE INVENTION

The present invention provides compositions for cancer immunotherapy and builds upon previous work by Bilodeau et. al., in WO2014/106208, the contents which are incorporated by reference in their entirety. The compositions include conjugates and nanoparticles, useful for the production of cancer vaccines, and activated T cells for adoptive cellular immunotherapy.

The conjugates of the present invention are constructed to compromise a targeting moiety, a linker and an active agent. In some embodiments, the targeting moiety may specifically target to a tumor cell or an immune cell. The active agent may comprise any agent that can manipulate cancer-specific immune responses positively, such as tumor associated antigens, agents that can enhance antigen presentation by antigen presenting cells such as dendritic cells, agents that can stimulate activation of cancer specific T cells, antibodies, and cytokines, chemokines and other immunoregulatory molecules.

### DETAILED DESCRIPTION OF THE INVENTION

The details of one or more embodiments of the invention are set forth in the accompanying description below. Although any materials and methods similar or equivalent to those described herein can be used in the practice or testing of the present invention, the preferred materials and methods are now described. Other features, objects and advantages of the invention will be apparent from the description. In the description, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present description will control.

A variety of strategies have been developed to elicit cancer specific immune responses. These strategies are developed to increase tumor antigen presentation by antigen presenting cells (APCs, e.g., dendritic cells), or to enhance cancer specific T cell proliferation, migration and cytotoxic function, or to increase cytokine mediated defensive mechanisms, or to modulate the immunosuppressive tumor microenvironment, or in combination of two or more different strategies to increase the efficacy of immunotherapy.

The present conjugates provide platforms for cancer immunotherapy modalities. The conjugate comprise three moieties: an active agent, a targeting moiety and a linker that connects the active agent and the targeting moiety. The active agent may be an agent that can stimulate/increase a cancer specific immune response. Examples of such agents include antibodies specific to a tumor antigen; tumor antigenic peptides (i.e., epitopes) that can increase the antigen presentation to T cells; agents that can stimulate proliferation (e.g., cytokines), expansion, maturation and migration of antigen presenting cells (e.g., dendritic cells), and/or increase antigen capture and processing in antigen presenting cells; agents that enhance cancer specific T cells expansion, proliferation and migration, and/or increase antigen recognition; cytokines and chemokines that positively regulate immune responses; or agents that can inhibit immunosuppressive signals in the tumor tissues.

The targeting moiety of the conjugate can function to deliver an active agent of the conjugate to a targeted area such as a tumor tissue or lymph node, or a type of cell of interest such as T cells, dendritic cells and/or NK cells. In some cases, the targeting moiety itself may have an immune stimulating activity, the same or different from the active agent in the same conjugate. The linker of the conjugate not only connects the active moiety and the targeting moiety, in some cases, but may also control/assist in the release of the active agent to a targeted area or a cell. It some aspects, it may provide a sustained release of the active agent for a period of time.

Design of the present conjugates is flexible and may be configured in various combinations depending on types, origins, metastatic status, and other clinical and pathological status of the cancers to be related. In some embodiments, one or more active agents from the same category such as different tumor antigen peptides from one common tumor associated antigen protein, or from different tumor associated antigen proteins but associated with one type of tumor; or from a combination of tumor associated antigens isolated from a single patient, i.e. personalized, may be connected to a targeting moiety through the linker in a conjugate.

In other embodiments, active agents may function through different mechanisms. Two or more active agent such as tumor specific antigenic epitopes and agents for increasing dendritic cell antigen capture may be connected in one conjugate. In another example, one or more tumor specific antigenic peptide, and one or more immune costimulatory molecule agonists may be included in one conjugate to increase the efficacy of tumor specific T cell activation.

In some embodiments, more than one targeting moiety may be linked to active agents of the conjugate for targeting different tissues, cells or even different intracellular components such as those of the cell surface and cytoplasm.

In addition to the conjugate itself, the present invention also provide particles, nanoparticles and/or polymeric nanoparticles that can encapsulate one or more conjugates of the present invention, providing an improved nanodelivery system. The present nano-delivery system improves pharmacokinetics, targeting of tissues and cells to enhance efficacy, specificity and lower toxicity. The present conjugates designed for increasing immune response, and particles comprising such conjugates provide more specific compositions and methods to fight cancer. APCs such as macrophages are good at phagocytosis and may be stimulated by nanoparticles. The active agents of the conjugates in the nanoparticle are then released inside the APCs. In some embodiments, the active agents are only released within certain environments, such as with the presence of lysozymes. In some embodiments, particles, nanoparticles and/or polymeric nanoparticles target bone marrow and delivers conjugates to bone marrow. Such solid nanoparticles and their preparation are taught in, for example, WO2014/106208, the contents of which are incorporated herein in their entirety.

### Definitions

The terms used in this invention are, in general, expected to adhere to standard definitions generally accepted by those having ordinary skill in the relevant art.

About: As used herein, the term “about” means a range of normal tolerance in the art, for example within 2 standard deviations of the mean. About can be understood as within 50%, 45%, 40%, 35%, 30%, 25%, 20%, 15%, 10%, 9%, 8%, 7%, 6%, 5%, 4%, 3%, 2%, 1%, 0.5%, 0.1%, 0.05%, or 0.01% of the stated value.

Administration: As used herein, the term “administration” means the actual physical introduction of the composition into or onto (as appropriate) the host. Any and all methods of introducing the composition into the host are contemplated according to the invention; the method is not dependent on any particular means of introduction and is not to be so construed. Means of introduction are well-known to those skilled in the art, and also are exemplified herein

Adoptive cellular immunotherapy: As used herein, the terms “adoptive cellular immunotherapy” or “adoptive immunotherapy” or “T cell immunotherapy”, or “Adoptive T cell therapy (ACT)”, are used interchangeably. Adoptive immunotherapy uses T cells that a natural or genetically engineered reactivity to a patient's cancer are generated in vitro and then transferred back into the cancer patient. The injection of a large number of activated tumor specific T cells can induce complete and durable regression of cancers.

Agonist: As used herein, the term “agonist” refers to any substance that binds to a target (e.g. a receptor); and activates or increases the biological activity of the target. For example, an “agonist” antibody is an antibody that activates or increases the biological activity of the antigen(s) it binds.

Antagonist: As used herein, the term “antagonist” refers to any agent that inhibits or reduces the biological activity of the target(s) it binds. For example, an “antagonist” antibody is an antibody that inhibits or reduces biological activity of the antigen(s) it binds.

Antigen: As used herein, the terms “antigen” or “immunogen,” as being used interchangeably, is defined as a molecule that provokes an immune response when it is introduced into a subject or produced by a subject such as tumor antigens which arise by the cancer development itself. This immune response may involve either antibody production, or the activation of specific immunologically-competent cells such as cytotoxic T lymphocytes and T helper cells, or both. An antigen can be derived from organisms, subunits of proteins/antigens, killed or inactivated whole cells or lysates. The term “antigenic” or “immunogenic” refers to a structure that is an antigen. These terms are used interchangeably.

Antigen presenting cells (APCs): As used herein, the term “antigen presenting cells” refers to cells that process antigens and present peptide epitopes on the cell surface via MHC molecules; APCs include dendritic cells (DCs), Langerhans cells, macrophages, B cells, and activated T cells. Dendritic cells (DCs) and macrophages are antigen presenting cells in vivo. The dendritic cells are more efficient APCs than macrophages. These cells are usually found in structural compartments of the lymphoid organs such as the thymus, lymph nodes and spleen, and in the bloodstream and other tissues of the body as well.

Antibodies: As used herein, “antibodies” are specialized proteins called immunoglobulins (Igs) that specifically recognize and bind to specific antigens that caused their stimulation. Antibody production by B lymphocytes in vivo and binding to foreign antigens is often critical as a means of signaling other cells to engulf, kill or remove that substance that contains the foreign antigens from the body. An immunoglobulin is a protein comprising one or more polypeptides substantially encoded by the immunoglobulin kappa and lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Also subclasses of the heavy chain are known. For example, IgG heavy chains in humans can be any of IgG1, IgG2, IgG3 and IgG4 subclass.

Antibodies may exist as full length intact antibodies or as a number of well-characterized fragments produced by digestion with various peptidases or chemicals, such as F(ab′)2, a dimer of Fab which itself is a light chain joined to VH-CH1 by a disulfide bond; an Fab′ monomer, a Fab fragment with the hinge region; and a Fc fragment, a portion of the constant region of an immunoglobulin.

While various antibody fragments are defined in terms of the digestion of an intact antibody, one of skill will appreciate that any of a variety of antibody fragments may be synthesized de novo either chemically or by utilizing recombinant DNA methodology. Thus, the term antibody, as used herein also includes antibody fragments either produced by the modification of whole antibodies or synthesized de novo or antibodies and fragments obtained by using recombinant DNA methodologies. Recombinant antibodies may be conventional full length antibodies, antibody fragments known from proteolytic digestion, unique antibody fragments such as Fv or single chain Fv (scFv), domain deleted antibodies, and the like. An Fv antibody is about 50 Kd in size and comprises the variable regions of the light and heavy chain. A single chain Fv (“scFv”) polypeptide is a covalently linked VH::VL heterodimer.

An antibody may be a non-human antibody, a human antibody, a humanized antibody or a chimeric antibody. The “chimeric antibody” means a genetically engineered fusion of parts of a non-human (e.g., mouse) antibody with parts of a human antibody. Generally, chimeric antibodies contain approximately 33% non-human protein and 67% human protein. Developed to reduce the HAMA response elicited by non-human antibodies, they combine the specificity of the non-human antibody with the efficient human immune system interaction of a human antibody. A human antibody may be a “fully human” antibody. The terms “human” and “fully human” is used to label those antibodies derived from transgenic mice carrying human antibody genes or from human cells. To the human immune system, however, the difference between “fully human” “humanized”, and “chimeric” antibodies may be negligible or nonexistent and as such all three may be of equal efficacy and safety.

Autologous: As used herein, the term “autologous” is meant to refer to any material derived from the same individual to which it is later to be re-introduced into the individual.

Cancer: As used herein, the term “cancer” refers a broad group of various diseases characterized by the uncontrolled growth of abnormal cells in the body. Unregulated cell division and growth divide and grow results in the formation of malignant tumors that invade neighboring tissues and may also metastasize to distant parts of the body through the lymphatic system or bloodstream.

Combination therapy: As used herein, the term “combination therapy” means a therapy strategy that embraces the administration of therapeutic compositions of the present invention (e.g., conjugates comprising one or more neoantigens) and one or more additional therapeutic agents as part of a specific treatment regimen intended to provide a beneficial (additive or synergistic) effect from the co-action of these therapeutic agents. Administration of these therapeutic agents in combination may be carried out over a defined time period (usually minutes, hours, days, or weeks depending upon the combination selected). In combination therapy, combined therapeutic agent may be administered in a sequential manner, or by substantially simultaneous administration.

Compound: As used herein, the term “compound,” as used herein, is meant to include all stereoisomers, geometric isomers, tautomers, and isotopes of the structures depicted. In the present application, compound is used interchangeably with conjugate. Therefore, conjugate, as used herein, is also meant to include all stereoisomers, geometric isomers, tautomers, and isotopes of the structures depicted.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Compounds of the present disclosure also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge. Examples prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

Copolymer: As used herein, the term “copolymer” generally refers to a single polymeric material that is comprised of two or more different monomers. The copolymer can be of any form, such as random, block, graft, etc. The copolymers can have any end-group, including capped or acid end groups.

Cytokine: As used herein, the term “cytokine” refers to a substance secreted by certain cells of the immune system and has a biological effect on other cells. Cytokines can be a number of different substances such as interferons, interleukins and growth factors.

Cytotoxic agent: As used herein, the term “cytotoxic agent” means a substance that inhibits or prevents the function of cells and/or causes destruction of cells, such as radioactive isotopes, chemotherapeutic agents, and toxins.

Cytotoxic T cell: As used herein, the terms “cytotoxic T cell (TC)” or “cytotoxic T lymphocyte (CTL)”, or “T-killer cells”, or “CD8+ T-cell” or “killer T cell” are used interchangeably. This type of white blood cells are T lymphocytes that can recognize abnormal cells including cancer cells, cells that are infected particularly by viruses, and cells that are damaged in other ways and induce the death of such cells.

Epitope: As used herein, the term “epitope” means a small peptide structure formed by contiguous amino acids, or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and about 9, or about 8-15 amino acids. A T cell epitope means a peptide which can be bound by the MHC molecules of class I or II in the form of a peptide-presenting MHC molecule or MHC complex and then, in this form, be recognized and bound by native T cells, cytotoxic T-lymphocytes or T-helper cells, respectively.

Human Leukocyte Antigen (HLA): As used herein, the terms “Human Leokocyte Antigen (HLA)”, “HLA proteins”, “HLA antigens”, “Major Histocompatibility Complex (MHC)”, “MHC molecules”, or “MHC proteins” all refer to proteins capable of binding peptides resulting from the proteolytic cleavage of protein antigens and representing potential T-cell epitopes, transporting them to the cell surface and presenting them there to specific cells, in particular cytotoxic T-lymphocytes or T-helper cells. The major histocompatibility complex in the genome comprises the genetic region whose gene products expressed on the cell surface are important for binding and presenting endogenous and/or foreign antigens and thus for regulating immunological processes. The major histocompatibility complex is classified into two gene groups coding for different proteins, namely molecules of MHC class I and molecules of MHC class II. The molecules of the two MHC classes are specialized for different antigen sources. The molecules of MHC class I present endogenously synthesized antigens, for example viral proteins and tumor antigens. The molecules of MHC class II present protein antigens originating from exogenous sources, for example bacterial products. The cellular biology and the expression patterns of the two MHC classes are adapted to these different roles.

MHC class I molecules (called HLA class I in human) consist of a heavy chain and a light chain and are capable of binding a short peptide with suitable binding motifs, and presenting it to cytotoxic T-lymphocytes. The peptide bound by the MHC molecules of class I originates from an endogenous protein antigen. The heavy chain of the MHC molecules of class I is preferably an HLA-A, HLA-B or HLA-C monomer, and the light chain is β-2-microglobulin.

MHC class II molecules (called HLA class II in human) consist of an α-chain and a β-chain and are capable of binding a short peptide with suitable binding motifs, and presenting it to T-helper cells. The peptide bound by the MHC molecules of class II usually originates from an extracellular of exogenous protein antigen. The α-chain and the β-chain are in particular HLA-DR, HLA-DQ, HLA-DP, HLA-DO and HLA-DM monomers.

Immune cell: As used herein, the term “immune cell” refers to a cell that is capable of participating, directly or indirectly, in an immune response. Immune cells include, but are not limited to T-cells, B-cells, antigen presenting cells, dendritic cells, natural killer (NK) cells, natural killer T (NK) cells, lymphokine-activated killer (LAK) cells, monocytes, macrophages, neutrophils, granulocytes, mast cells, platelets, Langerhan's cells, stem cells, peripheral blood mononuclear cells, cytotoxic T-cells, tumor infiltrating lymphocytes (TIL), etc. “An antigen presenting cell” (APC) is a cell that are capable of activating T cells, and includes, but is not limited to, monocytes/macrophages, B cells and dendritic cells (DCs). “Dendritic cell” or “DC” refers to any member of a diverse population of morphologically similar cell types found in lymphoid or non-lymphoid tissues. These cells are characterized by their distinctive morphology, high levels of surface MHC-class II expression. DCs can be isolated from a number of tissue sources. DCs have a high capacity for sensitizing MHC-restricted T cells and are very effective at presenting antigens to T cells in situ. The antigens may be self-antigens that are expressed during T cell development and tolerance, and foreign antigens that are present during normal immune processes. As used herein, an “activated DC” is a DC that has been pulsed with an antigen and capable of activating an immune cell. “T-cell” as used herein, is defined as a thymus-derived cell that participates in a variety of cell-mediated immune reactions, including CD8+ T cell and CD4+ T cell. “B-cell” as used herein, is defined as a cell derived from the bone marrow and/or spleen. B cells can develop into plasma cells which produce antibodies.

Immune response: As used herein, the term “immune response” means a defensive response a body develops against “foreigner” such as bacteria, viruses and substances that appear foreign and harmful. An anti-cancer immune response refers to an immune surveillance mechanism by which a body recognizes abnormal tumor cells and initiates both the innate and adaptive of the immune system to eliminate dangerous cancer cells.

The innate immune system is a non-specific immune system that comprises the cells (e.g., Natural killer cells, mast cells, eosinophils, basophils; and the phagocytic cells including macrophages, neutrophils, and dendritic cells) and mechanisms that defend the host from infection by other organisms. An innate immune response can initiate the productions of cytokines, and active complement cascade and adaptive immune response. The adaptive immune system is specific immune system that is required and involved in highly specialized systemic cell activation and processes, such as antigen presentation by an antigen presenting cell; antigen specific T cell activation and cytotoxic effect.

Linker: As used herein, the term “linker” refers to a carbon chain that can contain heteroatoms (e.g., nitrogen, oxygen, sulfur, etc.) and which may be 1, 2, 3, 4, 5, 6, 7, 8, 9, 10, 11, 12, 13, 14, 15,16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 atoms long. Linkers may be substituted with various substituents including, but not limited to, hydrogen atoms, alkyl, alkenyl, alkynl, amino, alkylamino, dialkylamino, trialkylamino, hydroxyl, alkoxy, halogen, aryl, heterocyclic, aromatic heterocyclic, cyano, amide, carbamoyl, carboxylic acid, ester, thioether, alkylthioether, thiol, and ureido groups. Those of skill in the art will recognize that each of these groups may in turn be substituted. Examples of linkers include, but are not limited to, pH-sensitive linkers, protease cleavable peptide linkers, nuclease sensitive nucleic acid linkers, lipase sensitive lipid linkers, glycosidase sensitive carbohydrate linkers, hypoxia sensitive linkers, photo-cleavable linkers, heat-labile linkers, enzyme cleavable linkers (e.g., esterase cleavable linker), ultrasound-sensitive linkers, and x-ray cleavable linkers. Linkers may include any of those taught in, for example, WO2014/10628, the contents of which are incorporated herein by reference in their entirety.

Mean particle size: As used herein, the term “mean particle size” generally refers to the statistical mean particle size (diameter) of the particles in the composition. The diameter of an essentially spherical particle may be referred to as the physical or hydrodynamic diameter. The diameter of a non-spherical particle may refer to the hydrodynamic diameter. As used herein, the diameter of a non-spherical particle may refer to the largest linear distance between two points on the surface of the particle. Mean particle size can be measured using methods known in the art such as dynamic light scattering. Two populations can be said to have a “substantially equivalent mean particle size” when the statistical mean particle size of the first population of particles is within 20% of the statistical mean particle size of the second population of particles; for example, within 15%, or within 10%.

The terms “monodisperse” and “homogeneous size distribution,” as used interchangeably herein, describe a population of particles, microparticles, or nanoparticles all having the same or nearly the same size. As used herein, a monodisperse distribution refers to particle distributions in which 90% of the distribution lies within 5% of the mean particle size.

Peptide: As used herein, the term “peptide” refers to a molecule composed of a series of residues, typically L-amino acids, connected one to the other, typically by peptide bonds between the a-amino and carboxyl groups of adjacent amino acids. Peptide sometimes is used interchangeably with the term “polypeptide,” Polypeptides or peptides can be a variety of lengths, either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described. In some embodiments, peptides are less than 50 amino acids in length.

Targeting moiety: As used herein, the term “targeting moiety” refers to a moiety that binds to or localizes to a specific locale. The moiety may be, for example, a protein, nucleic acid, nucleic acid analog, carbohydrate, or small molecule. The locale may be a tissue, a particular cell type, or a subcellular compartment. In some embodiments, a targeting moiety can specifically bind to a selected component of the targeted locale.

Tumor associated antigen (TAA): As used herein, the term “tumor associated antigen (TAA)” refers to an antigenic substance produced in tumor cells. Tumor associated antigens may be encoded by a primary open reading frame of gene products that are differentially expressed by tumors, and not by normal tissues. They may also be encoded by mutated genes, intronic sequences, or translated alternative open reading frames, pseudogenes, antisense strands, or represent the products of gene translocation events. Tumor-associated antigens (TAA) can derive from any protein or glycoprotein synthesized by the tumor cell. TAA proteins can reside in any subcellular compartment of the tumor cell; i.e., they may be membrane-bound, cytoplasmic, nuclear-localized, or even secreted by the tumor cells. A TAA may allow for a preferential recognition of tumor cells by specific T cells or immunoglobulins, therefore activate an anti-tumor immune response to kill tumor cells.

Vaccine: As used herein, the term “vaccine” refers to a composition for generating immunity for the prophylaxis and/or treatment of diseases.

### Compositions of the Invention

Compositions of the present inventions include conjugates comprising a targeting moiety, a linker, and one or more active agents, e.g., one or more immuno-oncological agents conjugated to the targeting moiety through a linker. Nanoparticles that package one or more such conjugates are also provided. The conjugates can be encapsulated into nanoparticles or disposed on the surface of the particles. In particular, conjugates of the present invention and nanoparticles comprising such conjugates may be used as immuno-oncological agents such as cancer vaccines, or as adjuvants to enhance anti-cancer immune responses in combination with other immunotherapies, or to generate cancer vaccines in vitro for in vivo cellular immunotherapy. The conjugates, nanoparticles comprising the conjugates, and/or formulations thereof can provide improved temporospatial delivery of the active agent and/or improved biodistribution compared to delivery of the active agent alone.

Conjugates, nanoparticles and other compositions of the present invention provide a system that is flexible in tailoring the composition and numbers of active agents (e.g., flexible addition and subtraction of active agents connected to the targeting moiety) important for harnessing an anti-tumor immune response, for example, antigen specific T cell activation and response.

Conjugates, nanoparticles and other compositions of the present invention may provide increased targeting properties since the targeting moieties of the conjugates specifically target to a selected tissue and/or certain types of cells of interest.

Conjugates, nanoparticles and other compositions of the present invention may coordinate action of the innate and adaptive phases of the immune system to produce an effective anti-cancer immune response. In some instances, they may be used for active immunotherapy and adoptive immunotherapy of cancer and/or other diseases (e.g., viral infection).

In one embodiment of the present invention, conjugates, nanoparticles and other compositions comprising conjugates may include a B cell immune response in subject.

In another embodiment of the present invention, conjugates, nanoparticles and other compositions comprising conjugates may include a CD4+ T cell immune response in a subject.

In further another embodiment of the present invention, conjugates, nanoparticles and other compositions comprising conjugates may induce a CD8+ T cell immune response in a subject.

In further another embodiment, conjugates, nanoparticles and other compositions of the present invention may also be used for in vivo and ex vivo activation and expansion of lymphocytes including T cells to elicit an anti-tumor immune response.

### I. Conjugates of the Invention

In accordance with the present invention, conjugates comprise at least three moieties: a targeting moiety (or ligand), a linker, and an active agent called a payload that is connected to the targeting moiety via the linker. In some embodiments, the conjugate may be a conjugate between a single active agent and a single targeting moiety with the formula: X—Y—Z, wherein X is the targeting moiety; Y is a linker; and Z is the active agent. In certain embodiments, One targeting moiety can be conjugated to two or more payloads wherein the conjugate has the formula: X—(Y—Z)ₙ. In certain embodiments, one active payload can be linked to two or more targeting ligands wherein the conjugate has the formula: (X—Y)ₙ—Z. In other embodiments, one or more targeting ligands may be connected to one or more active payloads wherein the conjugate formula may be (X—Y—Z)ₙ. In various combinations, the formula of the conjugates maybe, for example, X—Y—Z—Y—X, (X—Y—Z)ₙ—Y—Z, or X—Y—(X—Y—Z)ₙ, wherein X is a targeting moiety; Y is a linker; Z is an active agent. The number of each moiety in the conjugate may vary dependent on types of agents, sizes of the conjugate, delivery targets, particles used to packaging the conjugate, other active agents (e.g., immunologic adjuvants) and routes of administration. Each occurrence of X, Y, and Z can be the same or different, e.g. the conjugate can contain more than one type of targeting moiety, more than one type of linker, and/or more than one type of active agent. n is an integer equal to or greater than 1. In some embodiments, n is an integer between 1 and 50, or between 2 and 20, or between 5 and 40. In some embodiments, n may be an integer of 2, 3, 4, 5, 6, 7, 8. 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 41, 43, 44, 45, 46, 47, 48, 49 or 50.

In some embodiments, the conjugate may comprise pendent or terminal functional groups that allow further modification or conjugation. The pendent or terminal functional groups may be protected with any suitable protecting groups.

Conjugates of the present invention may target discrete pathways involved in critical processes of anti-cancer immune responses. These critical processes may include antigen degradation and processing, activation of dendritic cells to present antigenic epitopes, production of cytokines (e.g., interferons), expression of co-stimulatory ligands, induction of a productive T cell response for example within lymph nodes, migration of activated T cells to the tumor microenvironment in response to chemokines and homing receptor expression, having effector T cells (e.g., CD4+ T cells and CD8+ T cells) gain access to antigen expressing tumor cells and maintenance of sufficient functionality of effector T cell to destroy tumor cells. For example, cancer antigens, as payloads of the conjugates, may be delivered to antigen presenting cells (APCs) (e.g., dendritic cells) through a targeting moiety with increased targeting delivery, therefore, enhancing the immunogenicity of TAAs to induce TAA specific cytotoxic T-lymphocytes (CTL).

In some embodiments, the conjugate comprises a payload that binds to a chimeric antigen receptor (CAR) T cell, a linker, and a targeting moiety that binds to a tumor cell. For example, the targeting moiety may bind to a cell surface protein on tumor cells, such as but not limited to a folate receptor, a somatostatin receptor (SSTR), or a luteinizing hormone-releasing hormone receptor (LHRHR). The payload may be a single chain variable fragment (scFV) that binds to a cell surface protein on CAR T cells.

### A. Payloads

### 1. Tumor Associated Antigens (TAAs) and Antigenic Peptides

As used herein, the terms “payload” and “active agent” are used interchangeably. Payload may be any active agents such as therapeutic agents, prophylactic agents, or diagnostic /prognostic agents. A payload may have a capability of manipulating a physiological function (e.g., anti-cancer immune response) in a subject. One or more, either the same or different payloads may be included in the present conjugate.

In accordance with the present invention, a payload may be an active agent that can boost or provoke an anti-cancer immune response in a subject. Immunotherapy is an advantageous strategy to treat cancer. Any compound that can provoke and/or enhance an immune response to destroy tumor cells in a subject may be included in the present conjugate. Such agents may be tumor associated antigens (TAAs), antigen epitopes including antigen peptides presented by either MHC (major histocompatibility complex) class I or MHC class II molecules; cytokines, chemokines, other immunomodulators, T cell receptors (TCRs), CD (cell differentiation molecules) antigens, antibodies, cytotoxic agents, cell adhesion molecules and any components that are involved in an immune response; or variants thereof. A payload may be a protein including a peptide, a nucleic acid, a sugar, a lipid, a lipoprotein, a glycoprotein, a glycolipid, or a small molecule.

In embodiments that a plural of payloads are included in one conjugate, the plural payloads may belong to the same category such as multiple epitope peptides derived from a single TAA, or multiple different tumor associated antigens isolated from a tumor tissue. In other aspects, a plural of payloads having different functionality such as a mix of tumor associated antigens and co-stimulatory factors may be included in the same conjugate to synergistically enhance the antigen presentation to T cells.

The initiation of an immune response against diseased tumor cells involves presenting a tumor specific antigen to the immune system. It has been known that tumor cells express specific antigens that are not normally expressed by normal cells. Many tumor associated antigens (TAAs) have been identified and antigenic peptides (epitopes) (either MHC class I specific or MHC class II specific) are isolated that can specifically activate an immune response (e.g., cytotoxic T lymphocyte response/CTL response) to attack abnormal tumor cells and promote their lysis in vivo. TAAs and epitope peptides derived from TAAs can be selected as antigens to selectively stimulate cytotoxic T lymphocyte (CTL) response. The ability of a TAA or a TAA peptide to induce CTL response depends on its ability to bind to specific MHC molecules and its ability to break immune tolerance.

There are two types of MHC/HLA molecules used for presenting antigens. (e.g.,TAAs) MHC/HLA class I molecules are expressed on the surface of all cells and MHC/HLA class II are expressed on the surface of professional antigen presenting cells (APCs). MHC/HLA class II molecules bind primarily to peptides derived from proteins made outside of an APC, but can present self (endogenous) antigens. In contrast, HLA class I molecules bind to peptides derived from proteins made inside a cell, including proteins expressed by an infectious agent (e.g., such as a virus) in the cell and by a tumor cell. When the HLA class I proteins reach the surface of the cell these molecules will thus display any one of many peptides derived from the cytosolic proteins of that cell, along with normal “self” peptides being synthesized by the cell. Peptides presented in this way are recognized by T-cell receptors which engage T-lymphocytes in an immune response against the antigens to induce antigen-specific cellular immunity.

In accordance with the present invention, a payload may be a TAA or an antigenic peptide (epitope) derived from a TAA. An antigenic peptide may be a CD8+ T cell epitope that binds to specific MHC (HLA in human) class I molecules with a high affinity. An antigenic peptide may be a CD4+ T cell epitope that binds to specific MHC (HLA in human) class II molecules with a high affinity. The antigenic peptide may be about 5 to 50 amino acids in length. The antigenic peptide may be greater than 5 amino acids in length, or greater than 10 amino acids in length, or greater than 15 amino acids in length, or greater than 20 amino acids in length, or greater than 25 amino acids in length, or greater than 30 amino acids in length, or greater than 35 amino acids in length, or greater than 40 amino acids in length, or greater than 45 amino acids in length. For example, the antigenic peptide may contain 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids. It is generally preferable that the antigenic peptide be as small as possible while still maintaining substantially all of the immunologic activity of the native protein. In some aspects, the HLA class I binding antigenic peptides (epitopes) may have a length of about 6 to about 15 amino acid residues, for example, 6, 7, 8, 9, 10, 11, 12, 13, 14 or 15. In other aspects, the HLA class II binding peptides (epitopes) may have about 6 to about 30 amino acid residues, e.g., 6, 7, 8, 9, 10, 11, 12, 13, 14, 15, 16, 17, 18, 19, 20, 21, 22, 23, 24, 25, 26, 27, 28, 29 or 30 amino acids, preferably to between about 13 and about 20 amino acids, e.g., 13, 14, 15, 16, 17, 18, 19 or 20 amino acids.

In some embodiments, the antigenic epitope from a TAA may be an epitope that induces a B cell response in a subject to generate TAA specific antibody mediated immune responses.

TAAs or TAA derived antigenic peptides may be delivered directly to activate T cells through the targeting moieties of the conjugate. Conjugates of the present invention comprising one or more TAAs and/or antigenic peptides derived from TAAs may provide vaccine platforms that can enhance immunogenicity and reduce toxicity such as autoimmune toxicity.

A TAA payload may be an oncofetal antigen that is typically only expressed at different stages during the development of the fetus and in cancerous somatic cells. Many proteins are normally expressed during fetal development but are transcriptionally repressed after birth or at early stage of infancy, therefore are not present, or are expressed in significantly lower levels in the corresponding normal adult tissue. Some of these developmental proteins are re-expressed in certain tumor cells and become oncofetal antigens. The oncofetal antigens have the potential to be used as tumor markers for diagnosis, treatment monitoring, follow-up after therapy and/or ultimately as targets for specific therapy of malignancy. Examples of oncofetal antigens may include, but are not limited to CEA (carcinoembryonic antigen) in colorectal carcinorma, iLRP/OFA (immature laminin receptor protein/oncofetal antigen) in renal cell carcinoma (RCC), TAG-72 (tumor associated glycoprotein-72) in prostate carcinoma, AFP (alpha-fetoprotein) in hepatocellular carcinoma (HCC), ROR1 (a receptor tyrosine kinase) in many malignant cells such as brain tumors, sperm protein 17, HMGA2 (high mobility group A2) in ovarian carcinoma, oncofetal H19, CR-1 (Cripto-1, a member of epidermal growth factor (EGF)-CFC family), trophoblast glycoprotein precursor and GPC-3 (Glypican-3, a member of heparan sulphate proteoglycans) in HCC. Some examples of T cell epitope peptides derived from oncofetal antigens may be used as payloads, such as those peptides disclosed in U.S. Pat. Nos. 7,718,762; 7,968,097; 7,994,276; 8,080,634; 8,669,230; 8,709,405; and U.S. patent publication NO: 2007/0049960; each of which is incorporated herein by reference in their entirety.

A TAA payload may be an oncoviral antigen that is encoded by tumorigenic transforming viruses (also called oncogenic viruses). Oncogenic viruses, when they infect host cells, can insert their own DNA (or RNA) into that of the host cells. When the viral DNA or RNA affects the host cell's genes, it can push the cell toward becoming cancer. Oncogenic viruses include, but are not limited to, RNA viruses, such as Flaviviridae and Retroviridae, and DNA viruses, such as Hepadnaviridae, Papovaviridae, specifically Papillomaviruses, Adenoviridae, Herpesviridae, and Poxviridae. Some examples of commonly known oncoviruses include human papilloma viruses (HPVs) which are main causes of cervical cancer, Epstein-Barr virus (EBV) which may cause nasopharyngeal cancer, certain types of fast-growing lymphomas (e.g., Burkitt lymphoma) and stomach cancer, hepatitis B, C and D viruses (HBV, HCV and HDV) in hepatocellular carcinoma (HCC), human immunodeficiency virus (HIV) which increases the risk of getting many typese of cancer (e.g., liver cancer, anal cancer and Hodgkin cancer), Kaposi sarcoma herpes virus (KSHV; also known as human herpes virus 8 (HHV8)) which is linked to lymphoma, human T-lymphotrophic virus (HTLV-1) and merkel cell polymavirus (MCV).

A viral antigen can be any defined antigen of a virus that is associated with a cancer in a human. A viral antigen is one that results in a CD8+ T-cell response that can be readily/easily measured. Desirably, the viral antigen is one to which an immune response can be induced or stimulated in a human and is universally recognized. Examples of suitable EBV antigens include, but are not limited to, Epstein-Barr nuclear antigen-1 (EBNA1), latent membrane protein 1 (LMP1), or latent membrane protein 2 (LMP2). Examples of suitable HPV antigens for conjugates include, but are not limited, L1 and L2 protein, and E5, E6, and E7. Examples of suitable KSHV antigens for conjugates may include but are not limited to, latency nuclear antigen (LANA) and v-cyclin. Examples of suitable HIV antigens include, but are not limited to gp160, gp120 and gag protein. It is within the scope of the present invention that any antigenic peptides derived from oncoviral antigens may be used as active payloads of the present conjugates.

A TAA payload may be an overexpressed or accumulated antigen that is expressed by both normal and neoplastic tissue, with the level of expression highly elevated in cancer tissues. Numerous proteins (e.g. oncogenes) are up-regulated in tumor tissues, including but not limited to adipophilin, AIM-2, ALDH1A1, BCLX(L), BING-4, CALCA, CD45, CD274, CPSF, cyclin D1, DKK1, ENAH, epCAM, ephA3, EZH2, FGF5, G250, HER-2/neu, HLA-DOB, Hepsin, IDO1, IGFB3, IL13Ralpha2, Intestinal carboxyl esterase, kallikrein 4, KIF20A, lengsin, M-CSF, MCSP, mdm-2, Meloe, Midkine, MMP-2, MMP-7, MUC-1, MUC5AC, p53, Pax5, PBF, PRAME, PSMA, RAGE-1, RGS5, RhoC, RNF43, RU2A5, SECERNIN 1, SOX10, STEAP1, survivin, Telomerase, TPBG, VEGF, and WT1.

Antigenic peptides derived from TAAs that are overexpressed in tumor tissues can be found in many references. Some examples may be U.S. Pat. No. 7,371,840; 7, 906, 620; U.S. patent publication No. 2010/0074925; the content of each of which is incorporated herein in their entirety.

A TAA payload may be a cancer-testis antigen that is expressed only by cancer cells and adult reproductive tissues such as testis and placenta. A TAA in this category may include, but are not limited to antigens from BAGE family, CAGE family, HAGE family, GAGE family, MAGE family (e.g., MAGE-A1, MAGE-A2, MAGE-A3, MAGE-A6 and MAGE-A13), SAGE family, XAGE family, MCAK, NA88-A (cancer/testis antigen 88), PSAD1, SSX-2, and SLLP-1. As a non-limiting example, NY-ESO-1 is one of the most immunogenic TAAs which expression is limited to testis in healthy subjects, but often overexpressed in various cancers such as HCC, melanoma, ovarian, and breast cancer.

A TAA payload may be a lineage restricted antigen that is expressed largely by a single cancer histotype. A lineage restricted antigen may include, but are not limited to, Melan-A/MART-1, Gp100/pmel17, Tyrosinase, TRP-1/-2, P.polypeptide, MC1R in melanoma; and prostate specific antigen (PSA) in prostate cancer. Any antigenic peptides derived from these TAAs may be used as active payloads of the present conjugates.

A TAA payload may be a mutated antigen that is only expressed by tumor cells as a result of genetic mutations or alterations in transcription. The antigen may be resulted from genetic substitution, insertion, deletion or any other genetic changes of a native cognate protein (i.e. a molecule that is expressed in normal cells). A subset of these mutations can alter protein coding sequences, therefore creating novel, foreign antigens: tumor neoantigen. As used herein, the term “tumor neoantigens” refers to tumor antigens that are present in tumor cells but not normal cells and do not induce deletion of their cognate antigen specific T cells in thymus (i.e., central tolerance). These tumor neoantigens may provide a “foreign” signal, similar to pathogens, to induce an effective immune response needed for cancer immunotherapy. A neoantigen may be restricted to a specific tumor. A neoantigen be a peptide/protein with a missense mutation (missense neoantigen), or a new peptide with long, completely novel stretches of amino acids from novel open reading frames (neoORFs). The neoORFs can be generated in some tumors by out-of-frame insertions or deletions (due to defects in DNA mismatch repair causing microsatellite instability), gene-fusion, read-through mutations in stop codons, or translation of improperly spliced RNA (e.g., Saeterdal et al., Frameshift-mutation-derived peptides as tumor-specific antigens in inherited and spontaneous colorectal cancer, Proc Natl Acad Sci USA, 2001, 98: 13255-13260). Studies have showed that neoORFs generated by frameshift mutations, which are not subject to central tolerance, induce highly specific antitumor immunity, and are thus highly valuable as antigens for cancer immunotherapy.

A series of murine and human studies have revealed that various gene products with missense mutations can encode peptides recognized by cognate cytotoxic T lymphocytes (CTLs) (Sensi and Anichini, Unique tumor antigens: evidence for immune control of genome integrity and immunogenic targets for T cell-mediated patent-specific immunotherapy. Clin Cancer Res., 2006, 12: 5023-5032). As non-limiting examples, these neoantigens may include mutated new peptides derived from alpha-actinin-4, ARTC1, BCR-ABL fusion protein (b3a2), B-RAF, CASP-5, CASP-8, beta-catenin, Cdc27, CDK4, CDKN2A, CLPP, CML-66, COA-1, connexin 37, dek-can fusion protein, EFTUD2, Elongation factor 2, ETV6-AML1 fusion protein, fibronectin, FLT3-ITD, FN1, GPNM8, LDLR-fucosyltransferase AS fusion protein, HLA-A2, HLA-A11, Hsp-70-1B, MART-2, ME1, MUM-1, MUM-2, MUM-3, Myosin class I, NFYC, neo-PAP, OGT, OS-9, p53, pml-RARalpha fusion protein, PRDX5, PTPRK, K-Ras, N-Ras, RBAF600, sirtuin-2, SNRPD1, SYT-SSX1/SSX2 fusion protein, TGF-beta receptor II, etc.

Additional neoantigen peptides may include SF3B1 peptides, MYD peptides, TP53 peptides, Abl peptides, FBXW7 peptides, MAPK peptides, and GNB1 peptides disclosed in US patent publication NO.: 20110293637; the content of which in incorporated herein in its entirety.

Tumor associated mutations are discovered rapidly through DNA and RNA sequencing of tumor and normal tissues. Massively parallel sequencing techniques can sequence the entire genome or exome of tumor and matched normal cells to identify all of the mutations that have occurred in tumor cells. The comprehensive maps of mutated antigens in tumor genomes bring new targets for therapeutic or prophylactic vaccines (Wood L D, et al., The genomic landscapes of human breast and colorectal cancers. Science, 2007, 318:1108-1113; PCT patent publication NO.: WO2014168874; the content of each of which is incorporated by reference in their entirety). In addition to de novo sequencing of tumor genomes to identify tumor specific mutations, many algorithms (e.g., NetMHC, IEDB) are applied to identify potential antigenic peptides (epitopes) generated by these mutations by predicting peptides binding to the cleft of patient-specific HLA (human leukocyte antigen) class I and class II molecules. (Castle et al., Exploiting the mutanome for tumor vaccination. Cancer Res, 2012, 72: 1081-1091).

Accordingly, these new neoantigens identified through large-scale sequencing and algorithm calculation may be linked to conjugates of the present invention as payloads. Novel tumor antigenic peptides are identified by some studies may be used as payloads of the conjugates. See, e.g., Nishimura et al., Cancer immunotherapy using novel tumor associated antigenic peptides identified by genome-wide cDNA microarray analyses, Cancer Sci. 2015, 106(5): 505-511; and Linnemann et al., high-throughput epitope discovery reveals frequent recognition of neo-antigens by CD4+ T cells in human melanoma, Nat. Med., 2015, 21(1): 81-85; the content of each of which is incorporated by reference in their entirety. Conjugates comprising tumor neoantigens may be used as ideal therapeutic and prophylactic vaccines.

A TAA payload may be an idiotypic antigen that is generated from highly polymorphic genes where a tumor cell expresses a specific “clonotype”, i.e., as in B cell, T cell lymphoma/leukemia resulting from clonal aberrancies, such as Immunoglobulin and T cell receptors (TCRs). Idiotypic antigens are a class of nonpathogen-associated neoantigens. For example, the malignant B cells express rearranged and multiply mutated surface immunoglobulins (Ig). Tumor specific idiotypes (e.g., immunoglobulin idiotypes) are regarded as particularly attractive tumor-specific antigens that can be successfully targeted by immunotherapy (e.g., Alejandro et al., Idiotypes as immunogens: facing the challenge of inducing strong therapeutic immune responses against the variable region of immunoglobulins, Front Oncol., 2012, 2: 159

A TAA payload may be a post-translationally altered antigen due to tumor -associated alterations in glycosylation, and other posttranslational modifications. Some examples may include MUC1 in colorectal carcinoma.

Some examples of antigenic peptides and their corresponding genes/proteins, HLA subtypes to which an antigenic peptide binds and tumors associated with them are listed in Table 1 (e.g., Vanern et al., Database of T cell defined human tumor antigens: the 2013 update, Cancer Imus. 2013, 13: 15).

TABLE 1
Examples of peptide antigen epitopes

| | | | | |
|---|---|---|---|---|
| Gene/Protein | Peptide | Position | HLA | Associated tumor |

Tumor antigens resulting from Mutations

| | | | | |
|---|---|---|---|---|
| α-actinin-4 | FIASNGVKLV | 118-127 | A2 | Lung carcinoma |
| ARTC1 | YSVYFNLPADTIYTN* | | DR1 | melanoma |
| BCR-ABL | SSKALQRPV | 926-934 | A2 | Chronic myeloid |
| fusion protein | GFKQSSKAL | 922-930 | B8 | leukemia |
| (b3a2) | ATGFKQSSKALQRPVAS | 920-936 | DR4 | |
| | ATGFKQSSKALQRPVAS | 920-936 | DR9 | |
| B-RAF | EDLTVKIGDFGLATEKSRWSGS | 586-614 | DR4 | melanoma |
| | HQFEQLS | | | |
| CASP-5 | FLIIWQNTM (frameshift product) | 65-75 | A2 | colorectal, gastric, and |
| | | | | endometrial carcinoma |
| CASP-8 | FPSDSWCYF | 476-484 | B35 | head and neck |
| | | | | squamous cell |
| | | | | carcinoma |
| B-catenin | SYLDSGIHF | 29-37 | A24 | melanoma |
| Cdc27 | FSWAMDLDPKGA (The mutation | 760-771 | DR4 | melanoma |
| | is not located in the region | | | |
| | encoding the peptide) | | | |
| CDK4 | ACDPHSGHFV | 23-32 | A2 | melanoma |
| CDKN2A | AVCPWTWLR (frameshift | 125-133 | A11 | melanoma |
| | product) | (p14ARF- | | |
| | | ORF3) | | |
| CLPP | ILDKVLVHL | 240-248 | A2 | melanoma |
| COA-1 | TLYQDDTLTLQAAG (The | 447-460 | DR4/ | colorectal carcinoma |
| | mutation is not located in the | | DR13 | |
| | region encoding the peptide) | | | |
| dek-can fusion | TMKQICKKEIRRLHQY | 342-357 | DR53 | Myeloid leukemia |
| protein | | | | |
| EFTUD2 | KILDAVVAQK | 668-677 | A3 | melanoma |
| Elongation factor | ETVSEQSNV | 581-589 | A68 | lung squamous |
| 2 | | | | carcinoma |
| ETV6-AML | RIAECILGM (not a naturally | 334-342 | A2 | acute lymphoblastic |
| fusion protein | processed peptide) | | | leukemia |
| | IGRIAECILGMNPSR | 332-346 | DP5/ | |
| | | | DP17 | |
| FLT3-ITD | YVDFREYEYY | 591-600 | A1 | acute lymphoblastic |
| | | | | leukemia |
| FN1 | MIFEKHGFRRTTPP | 2050-2063 | DR2 | melanoma |
| GPNMB | TLDWLLQTPK | 179-188 | A3 | melanoma |
| LDLR- | WRRAPAPGA | 315-323 | DR1 | melanoma |
| fucosyltransferase | PVTWRRAPA | 312-320 | DR1 | |
| AS fusion | | | | |
| protein | | | | |
| Hsp70-2 | SLFEGIDIYT | 286-295 | A2 | Renal cell carcinoma |
| | AEPINIQTW | 262-270 | B44 | Bladder tumor |
| MART2 | FLEGNEVGKTY | 446-455 | A1 | melanoma |
| ME1 | FLDEFMEGV | 224-232 | A2 | Non-small cell lung |
| | | | | carcinoma |
| MUM-1 | EEKLIVVLF | 30-38 | B44 | melanoma |
| MUM-2 | SELFRSGLDSY | 123-133 | B44 | melanoma |
| | FRSGLDSYV | 126-134 | Cw6 | |
| MUM-3 | EAFIQPITR | 322-330 | A68 | melanoma |
| Neo-PAP | RVIKNSIRLTL (The mutation is | 724-734 | DR7 | melanoma |
| | not located in the region | | | |
| | encoding the peptide) | | | |
| Myosin class 1 | KINKNPKYK | 911-919 | A3 | melanoma |
| NFYC | QQITKTEV | 275-282 | B52 | lung squamous cell |
| | | | | carcinoma |
| OGT | SLYKFSPFPL (frameshift product) | 28-37 | A2 | Colorectal carcinoma |
| OS-9 | KELEGILLL | 438-446 | B44 | melanoma |
| P53 | VVPCEPPEV | 217-225 | A2 | head and neck |
| | | | | squamous cell |
| | | | | carcinoma |
| Pml-RARα | NSNHVASGAGEAAIETQSSSSE | | DR11 | pro myelocytic |
| fusion protein | EIV | | | leukemia |
| PRDX5 | LLLDDLLVSI | 163-172 | A2 | melanoma |
| PTPRK | PYYFAAELPPRNLPEP | 667-682 | DR10 | melanoma |
| K-ras | VVVGAVGVG | 7-15 | B35 | pancreatic |
| | | | | adenocarcinoma |
| N-ras | ILDTAGREEY | 55-64 | A1 | melanoma |
| RBAF600 | RPHVPESAF | 329-337 | B7 | melanoma |
| SIRT2 | KIFSEVTLK | 192-200 | A3 | melanoma |
| SNRPD1 | SHETVIIEL | 11-19 | B38 | melanoma |
| SYT-SSX fusion | QRPYGYDQIM | 402-410 | B7 | Sarcoma |
| protein | | (SYT) | | |
| TGF-βRII | RLSSCVPVA (frameshift product) | A2 | 131-139 | Colorectal carcinoma |

Oncofetal antigens

| | | | | |
|---|---|---|---|---|
| α-fetoprotein | GVALQTMKQ | 542-550 | A2 | |
| | FMNKFIYEI | 158-166 | A2 | |
| | QLAVSVILRV | 364-373 | DR13 | |
| Glypican-3 | FVGEFFTDV | 144-152 | A2 | |
| | EYILSLEEL | 298-306 | A24 | |
| CEA | IMIGVLVGV | 691-699 | A2 | Gut carcinoma |
| | GVLVGVALI | 694-702 | A2 | |
| | HLFGYSWYK | 61-69 | A3 | |
| | QYSWFVNGTF | 268-277 | A24 | |
| | TYACFVSNL | 652-660 | A24 | |
| | AYVCGIQNSVSANRS | 568-582 | DR3 | |
| | DTGFYTLHVIKSDLVNEEATGQ | 116-140 | DR4 | |
| | FRV | | | |
| | YSWRINGIPQQHTQV | 625-639 | DR4 | |
| | TYYRPGVNLSLSC | 425-437 | DR7 | |
| | EIIYPNASLLIQN | 99-111 | DR7 | |
| | LWWVNNQSLPVSP | 177-189 | DR11/ | |
| | | and | DR13 | |
| | | 355-367 | | |

Cancer-testis antigens(shared tumor specific antigens)

| | | | |
|---|---|---|---|
| BAGE-1 | AARAVFLAL | 2-10 | Cw16 |
| GAGE-1,2,8 | YRPRPRRY | 9-16 | Cw6 |
| GAGE-3,4,5,6,7 | YYWPRPRRY | 10-18 | A29 |
| LAGE-1 | MLMAQEALAFL | ORF2 | A2 |
| | | (1-11) | |
| | SLLMWITQC | 157-165 | A2 |
| | ELVRRILSR | 103-111 | A68 |
| | APRGVRMAV | ORF2 | B7 |
| | | (46-54) | |
| | SLLMWITQCFLPVF | 157-170 | DP4 |
| | QGAMLAAQERRVPRAAEVPR | ORF2 | DR3 |
| | | (14-33) | |
| | AADHRQLQLSISSCLQQL | 139-156 | DR4 |
| | CLSRRPWKRSWSAGSCPGMPH | ORF2 | DR11/ |
| | L | (81-102) | DR12 |
| | AGATGGRGPRGAGA | 37-50 | DR15 |
| MAGE-A1 | EADPTGHSY | 161-169 | A1/B35 |
| | KVLEYVIKV | 278-286 | A2 |
| | SLFRAVITK | 96-104 | A3 |
| | RVRFFFPSL | 289-298 | B7/Cw7 |
| | REPVTKAEML | 120-129 | B37 |
| | KEADPTGHSY | 160-169 | B44 |
| | SAFPTTINF | 62-70 | Cw2 |
| | SAYGEPRKL | 230-238 | Cw3 |
| | TSCILESLFRAVITK | 90-104 | DP4 |
| | PRALAETSYVKVLEY | 268-282 | DP4 |
| | EYVIKVSARVRF | 281-292 | DR15 |
| MAGE-A2 | YLQLVFGIEV | 157-166 | A2 |
| | EYLQLVFGI | 156-164 | A24 |
| | EGDCAPEEK | 212-220 | Cw7 |
| | LLKYRAREPVTKAE | 121-134 | DR13 |
| MAGE-A3 | EVDPIGHLY | 168-176 | A2 |
| | KVAELVHFL | 112-120 | A2 |
| | FPDLESEF | 97-105 | A24 |
| | VAELVHFLL | 113-121 | A24 |
| | MEVDPIGHLY | 167-176 | B18 |
| | EVDPIGHLY | 168-176 | B35 |
| | AELVHFLLL | 114-122 | B40 |
| | MEVDPIGHLY | 167-176 | B44 |
| | WQYFFPVIF | 143-151 | B52 |
| | EGDCAPEEK | 212-220 | Cw7 |
| | KKLLTQHFVQENYLEY | 243-258 | DP4, |
| | | | DQ6 |
| | RKVAELVHFLLLKYR | 111-125 | DP4, |
| | | | DR4 |
| | ACYEFLWGPRALVETS | 267-282 | DR1 |
| | VIFSKASSSLQL | 149-160 | DR4, |
| | | | DR7 |
| | VFGIELMEVDPIGHL | 161-175 | DR7 |
| | GDNQIMPKAGLLIIV | 191-205 | DR11 |
| | TSYVKVLHHMVKISG | 281-295 | DR11 |
| | FLLLKYRAREPVTKAE | 119-134 | DR13 |
| MAGE-A4 | EVDPASNTY | 169-177 | A1 |
| | GVYDGREHTV | 230-239 | A2 |
| | NYKRCFPVI | 143-151 | A24 |
| | SESLKMIF | 156-163 | B37 |
| MAGE-A6 | MVKISGGPR | 290-298 | A34 |
| | EVDPIGHVY | 168-176 | B35 |
| | REPVTKAEML | 127-136 | B37 |
| | EGDCAPEEK | 212-220 | Cw7 |
| | ISGGPRISY | 293-301 | Cw16 |
| MAGE-A9 | ALSVMGVYV | 223-231 | A2 |
| MAGE-A10 | GLYDGMEHL | 254-262 | A2 |
| | DPARYEFLW | 290-298 | B53 |
| | VRIGHLYIL | 170-178 | Cw7 |
| MAGE-C1 | ILFGISLREV | 959-968 | A2 |
| | KVVEFLAML | 1083-1091 | A2 |
| | SSALLSIFQSSPE | 137-149 | DQ6 |
| | SFSYTLLSL | 450-458 | DQ6 |
| | VSSFFSYTL | 779-787 | DR15 |
| MAGE-C2 | LLFGLALIEV | 191-200 | A2 |
| | ALKDVEERV | 336-344 | A2 |
| | SESIKKKVL | 307-315 | B44 |
| | ASSTLYLVF | 42-50 | B57 |
| | SSTLYLVFSPSSFST | 43-57 | DR15 |
| NA88-A | QGQHFLQKV | / | B13 |
| LAGE-2 | SLLMWITQC | 157-165 | A2 |
| (NY-ESO-1) | MLMAQEALAFL | ORF2 | A2 |
| | | (1-11) | |
| | YLAMPFATPME | 91-101 | A24 |
| | ASGPGGGAPR | 53-62 | A31 |
| | LAAQERRVPR | ORF2 | A31 |
| | | (18-27) | |
| | TVSGNILTIR | 127-136 | A68 |
| | APRGPHGGAASGL | 60-72 | B7 |
| | MPFATPMEA | 94-104 | B35 |
| | KEFTVSGNILTI | 124-135 | B49 |
| | MPFATPMEA | 94-102 | B51 |
| | FATPMEAEL | 96-104 | B52 |
| | FATPMEAELAR | 96-106 | C12 |
| | LAMPFATPM | 92-100 | Cw3 |
| | ARGPESRLL | 80-88 | Cw6 |
| | SLLMWITQCFLPVF | 157-170 | DP4 |
| | LLEFYLAMPFATPMEAELARRS | 87-111 | DP4, |
| | LAQ | | DR1 |
| | EFYLAMPFATPM | 89-100 | DR1 |
| | PGVLLKEFTVSGNILTIRLTAAD | 119-143 | DR1 |
| | HR | | |
| | RLLEFYLAMPFA | 86-97 | DR2 |
| | QGAMLAAQERRVPRAAEVPR | ORF2 | DR3 |
| | | (14-33) | |
| | PFATPMEAELARR | 95-107 | DR4 |
| | PGVLLKEFTVSGNILTIRLT | 119-138 | DR4 |
| | VLLKEFTVSG | 121-130 | DR4 |
| | AADHRQLQLSISSCLQQL | 139-156 | DR4 |
| | LLEFYLAMPFATPMEAELARRS | 87-111 | DR4, |
| | LAQ | | DR7 |
| | LKEFTVSGNILTIRL | 123-137 | DR5b |
| | PGVLLKEFTVSGNILTIRLTAAD | 119-143 | DR7 |
| | HR | | |
| | KEFTVSGNILT | 124-134 | DR8 |
| | LLEFYLAMPFATPM | 87-100 | DR9 |
| | AGATGGRGPRGAGA | 37-50 | DR15 |
| SAGE | LYATVIHDI | 715-723 | A24 |
| SSX-2 | KASEKIFYV | 41-49 | A2 |
| | EKIQKAFDDIAKYFSK | 19-34 | DP1 |
| | FGRLQGISPKI | 101-111 | DR1 |
| | WEKMKASEKIFYVYMKRK | 37-54 | DR3 |
| | KIFYVYMKRKYEAMT | 45-59 | DR4 |
| | KIFYVYMKRKYEAM | 45-58 | DR11 |
| SSX-4 | INKTSGPKRGKHAWTHRLRE | 151-170 | DP10 |
| | YFSKKEWEKMKSSEKIVYVY | 31-50 | DR3 |
| | MKLNYEVMTKLGFKVTLPPF | 51-70 | DR8 |
| | KHAWTHRLRERKQLVVYEEI | 161-180 | DR8, |
| | | | DR52 |
| | LGFKVTLPPFMRSKRAADFH | 61-80 | DR11 |
| | KSSEKIVYVYMKLNYEVMTK | 41-60 | DR15 |
| TAG-1 | SLGWLFLLL | 78-86 | A2 |
| | LSRLSNRLL | 42-50 | B8 |
| TRAG-3 | CEFHACWPAFTVLGE | 34-48 | DR1, |
| | | | DR4, |
| | | | DR7 |
| XAGE-1b | RQKKIRIQL | 21-29 | A2 |
| (GAGED2a) | HLGSRQKKIRIQLRSQ | 17-32 | DR4 |
| | CATWKVICKSCISQTPG | 33-49 | DR9 |

Antigens overexpressed in tumors

| | | | | Normal tissue |
|---|---|---|---|---|
| Gene/Protein | Peptide | Position | HLA | expression |
| Adipophilin | SVASTITGV | 129-137 | A2 | adipocytes, macrophages |
| ALDH1A1 | RSDSGQQARY | intron | A1 | mucosa, keratinocytes |
| CALCA | VLLQAGSLHA | 16-25 | A2 | Thyroid |
| CD45 | KFLDALISL | 556-564 | A24 | proliferating cells, |
| | | | | testis, multiple tissues |
| CD274 | LLNAFTVTV | 15-23 | A2 | multiple tissues (lung, |
| | | | | heart, dendritic cells, |
| | | | | etc.) and induced by |
| | | | | IFN-γ |
| CPSF | KVHPVIWSL | 250-258 | A2 | ubiquitous (low level) |
| | LMLQNALTTM | 1360-1369 | A2 | |
| Cyclin D1 | LLGATCMFV | 101-109 | A2 | ubiquitous (low level) |
| | NPPSMVAAGSVVAAV | 198-212 | DR4 | |
| DKK1 | ALGGHPLLGV | 20-29 | A2 | testis, prostate, |
| | | | | mesenchymal stem cells |
| ENAH | TMNGSKSPV | 502-510 | A2 | breast, prostate stroma |
| | | | | and epithelium of colon- |
| | | | | rectum, pancreas, |
| | | | | endometrium |
| EpCAM | RYQLDPKFI | 173-181 | A24 | Epithelial cells |
| EphA3 | DVTFNIICKKCG | 356-367 | DR11 | Many tissues |
| EZH2 | FMVEDETVL | 120-128 | A2 | ubiquitous (low level) |
| | FINDEIFVEL | 165-174 | A2 | |
| | KYDCFLHPF | 291-299 | A24 | |
| | KYVGIEREM | 735-743 | A24 | |
| FGF5 | NTYASPRFK | 172-176 | A3 | Brain and kidney |
| G250/CAIX | HLSTAFARV | 254-262 | A2 | stomach, liver, pancreas |
| HER-2/neu | KIFGSLAFL | 369-377 | A2 | Ubiquitous (low level) |
| | IISAVVGIL | 654-662 | A2 | |
| | ALCRWGLLL | 5-13 | A2 | |
| | ILHNGAYSL | 435-443 | A2 | |
| | RLLQETELV | 689-697 | A2 | |
| | VVLGVVFGI | 665-673 | A2 | |
| | HLYQGCQVV | 48-56 | A2 | |
| | YLVPQQGFFC | 1023-1032 | A2 | |
| | PLQPEQLQV | 391-399 | A2 | |
| | TLEEITGYL | 402-410 | A2 | |
| | ALIHHNTHL | 466-474 | A2 | |
| | PLTSIISAV | 650-658 | A2 | |
| | VLRENTSPK | 754-762 | A3 | |
| | TYLPTNASL | 63-71 | A24 | |
| HLA-DOB | FLLGLIFLL | 232-240 | A2 | B lymphocytes, |
| | | | | monocytes, blood cells |
| IDO1 | ALLEIASCL | 199-207 | A2 | lymph nodes, placenta, |
| | | | | and many cell types in |
| | | | | the course of |
| | | | | inflammatory response |
| IGF2B3 | NLSSAEVVV | 515-523 | A2 | Ubiquitous (low level) |
| | RLLVPTQFV | 199-207 | A3 | |
| IL13Rα | WLPFGFILI | 345-353 | A2 | |
| Kallikrein 4 | FLGYLILGV | 11-19 | A2 | prostate and ovarian |
| | SVSESDTIRSISIAS | 125-139 | DP4 | cancer |
| | LLANGRMPTVLQCVN | 155-169 | DR4 | |
| | RMPTVLQCVNVSVVS | 160-174 | DR7 | |
| KIF20A | LLSDDDVVV | 12-20 | A2 | ubiquitous (low level) |
| | AQPDTAPLPV | 284-293 | A2 | |
| | CIAEQYHTV | 809-817 | A2 | |
| Lengsin | FLPEFGISSA | 270-279 | A2 | eye lens and low level in |
| | | | | multiple tissues |
| M-CSF | LPAVVGLSPGEQEY | Alt ORF | B35 | liver and kidney |
| MCSP | VGQDVSVLFRVTGALQ | 693-708 | DR11 | endothelial cells, |
| | | | | chondrocytes, smooth |
| | | | | muscle cells |
| Mdm-2 | VLFYLGQY | 53-60 | A2 | brain, muscle and lung |
| Meloe | TLNDECWPA | 36-44 | A2 | ubiquitous (low level) |
| | ERISSTLNDECWPA | 31-44 | DQ2 | |
| | FGRLQGISPKI | 32-44 | DQ6 | |
| | TSREQFLPSEGAA | 11-23 | DR1 | |
| | CPPWHPSERISSTL | 24-37 | DR11 | |
| Midkine | ALLALTSAV | 13-21 | A2 | ubiquitous (low level) |
| | AQCQETIRV | 114-122 | A2 | |
| | LTLLALLALTSAVAK | 9-23 | DR4 | |
| MMP-7 | SLFPNSPKWTSK | 96-107 | A3 | ubiquitous (low level) |
| MUC1 | STAPPVHNV | 950-958 | A2 | glandular epithelia |
| | LLLLTVLTV | 12-20 | A2 | |
| | PGSTAPPAHGVT | repeated | DR3 | |
| MUC5AC | TCQPTCRSL | 716-724 | A24 | mucosal cells, respiratory |
| | | | | tract, and stomach |
| | | | | epithelia |
| p53 | LLGRNSFEV | 264-277 | A2 | ubiquitous (low level) |
| | RMPEAAPPV | | A2 | |
| | SQKTYQGSY | 99-107 | B46 | |
| | PGTRVRAMAIYKQ | 153-165 | DP5 | |
| | HLIRVEGNLRVE | 193-204 | DR14 | |
| PAX5 | TLPGYPPHV | 311-319 | A2 | hemopoietic system |
| PBF | CTACRWKKACQR | 499-510 | B55 | ovary, pancreas, spleen, |
| | | | | liver |
| PRAME | VLDGLDVLL | 100-108 | A2 | testis, ovary, |
| | SLYSFPEPEA | 142-151 | A2 | endometrium, adrenals |
| | ALYVDSLFFL | 300-309 | A2 | |
| | SLLQHLIGL | 425-433 | A2 | |
| PSMA | NYARTEDFF | 178-186 | A24 | prostate, CNS, liver |
| RAGE-1 | LKLSGVVRL | 352-360 | A2 | Retina |
| | SPSSNRIRNT | 11-20 | B7 | |
| RGS5 | LAALPHSCL | 5-13 | A2 | heart, skeletal muscle, |
| | GLASFKSFLK | 74-83 | A3 | pericytes |
| RhoC | RAGLQVRKNK | 176-185 | A3 | ubiquitous (low level) |
| RNF43 | ALWPWLLMAT | 11-20 | A2 | |
| | NSQPVVVLCL | 721-729 | A24 | |
| Secernin 1 | KMDAEHPEL | 196-204 | A2 | Ubiquitous |
| SOX10 | AWISKPPGV | 332-340 | A2 | ubiquitous (low level) |
| | SAWISKPPGV | 331-340 | A2 | |
| STEAP1 | MIAVFLPIV | 292-300 | A2 | Prostate |
| | HQQYFYKIP1LVINK | 102-116 | A2 | |
| Survivin | ELTLGEFLKL | 95-104 | A2 | Ubiquitous |
| | TLGEFLKLDRERAKN | 97-111 | DR1 | |
| Telomerase | RLVDDFLLV | 865-873 | A2 | testis, thymus, bone |
| | RPGLLGASVLGLDDI | 672-686 | DR7 | marrow, lymph nodes |
| | LTDLQPYMRQFVAHL | 766-780 | DR11 | |
| TPBG | RLARLALVL | 17-25 | A2 | multiple tissues |
| WT1 | TSEKRPFMCAY | 317-327 | A1 | testis, ovary, bone |
| | CMTWNQMNL | 235-243 | A24 | marrow, spleen |
| | LSHLQMHSRKH | 337-347 | DP5 | |
| | KRYFKLSBLQMHSRKH | 332-347 | DP5, | |
| | | | DR5 | |

Others

| | | | | |
|---|---|---|---|---|
| MART-1 | ILTVILGVL | 32-40 | A2 | Melanoma |
| | EAAGIGILTV | 26-35 | B35 | |
| | RNGYRALMDKS | 51-61 | Cw7 | |
| | YTTAEEAAGIGILTVILGVLLLI | 51-61 | DP5 | |
| | GCWYCRR | | | |
| | EEAAGIGILTVI | 25-36 | DQ6 | |
| | APPAYEKLpSAEQ | 100-111 | DR1 | |
| | RNGYRALMDKSLHVGTQCAL | 51-73 | DR4 | |
| | TRR | | | |
| | MPREDAHFIYGYPKKGHGHS | 1-20 | DR11 | |
| PAP | FLFLLFFWL | 18-26 | A2 | prostate cancer |
| | TLMSAMTNL | 112-120 | A2 | |
| | ALDVYNGLL | 299-307 | A2 | |
| PSA | FLTPKKLQCV | 165-174 | A2 | prostate carcinoma |
| | VISNDVCAQV | 178-187 | A2 | |
| RAB38 | VLHWDPETV | 50-58 | A2 | Melanoma |
| TRP-1 | MSLQRQFLR | Alt. ORF | A31 | Melanoma |
| | ISPNSVFSQWRVVCDSLEDYD | 277-297 | DR4 | |
| | SLPYWNFATG | 245-254 | DR15 | |
| | SQWRVVCDSLEDYDT | 284-298 | DR17 | |
| TRP-2 | SVYDFFVVVL | 180-188 | A2 | Melanoma |
| | TLDSQVMSL | 360-368 | A2 | |
| | LLGPGRPYR | 197-205 | A31 | |
| | LLGPGRPYR | 387-395 | Cw8 | |
| | QCTEVRADTRPWSGP | 60-74 | DR3 | |
| | ALPYWNFATG | 241-250 | DR15 | |
| Tyrosinase | KCDICTDEY | 243-251 | A1 | Melanoma |
| | SSDYVIPIGTY | 146-156 | A1 | |
| | MLLAVLYCL | 1-9 | A2 | |
| | CLLWSFQTSA | 8-17 | A2 | |
| | YMDGTMSQV | 369-377 | A2 | |
| | AFLPWHRLF | 206-214 | A24 | |
| | QCSGNFMGF | 90-98 | A26 | |
| | TPRLPSSADVEF | 309-320 | B35 | |
| | QNILLSNAPLGPQFP | 56-70 | DR4 | |
| | SYLQDSDPDSFQD | 450-462 | DR4 | |
| | FLLHHAFVDSIFEQWLQRHRP | 386-406 | DR15 | |

*The mutation creates a start codon (ATG) that opens an alternative ORF encoding the antigenic peptide. This peptide is recognized by regulatory T cells (Tregs).

Additionally, payloads of the present conjugates may be tumor specific antigens and/or their antigenic peptides disclosed in U.S. Pat. Nos. 8,961,985; 8,951,975; 8,933,014; 8,889,616; 8,895,514; 8,889,616; 8,871,719; 8,697,631; 8,669,230; 8,647,629; 8,653, 035; 8,569,244; 8,455,615; 8,492,342; 8, 318, 677; 8, 258, 260 ; 8,212,000; 8,211,999; 8,147,838; 8,119,139; 8,080,634; 8,067,529; 8,034,334; 8,007,810; 7,994,276; 7,939,627; 7,833,970; 7,833,969; 7,846,446; 7,807,642; 7,247,615; 6,063,900; U.S. Patent publication Nos.: 2015/0147347; 2015/0125477; 2015/0125478; 2015/0110797; 2015/0010587; 2014/0348902; 2014/0322253; 2014/0256648; 2014/0255437; 2014/0178409; 2014/0154281; 2013/0108664; 2012/0308590; 2011/0229504; 2011/0212116; 2011/0052614; PCT patent publication NOs.: WO2015/082499; WO2015/071763; WO2015/018805; WO2014/188721; WO2014/136453; WO2014/141683; WO2014/141652; WO2014/106886; WO2014/087626; WO2014/010232; WO2014/010231; WO2014/010229; WO2013/135553; WO2000023584; the content of each of which is herein incorporated by reference in their entirety.

Antigenic peptides may also include those identified by methods disclosed in, e.g., U.S. Pat. Nos. 9,090,322; 8,945,573; 8,883,164; and US patent publication NOs.: 2014/0370040; the content of each of which is herein incorporated by reference in their entirety.

Other potential TAAs and antigenic peptides may include those discussed by, e.g., Akiyama et al., Cancer Immunol. Immunother. 2012, 61: 2311-2319; Alisa et al., J Immunol 2008, 180: 5109-5117; Alves et al., Cancer Res 2003; 63: 8476-8480; Anderson et al., Cancer Res 2004, 64: 5456-5460; Bae et al., Br J Haematol 2012, 157: 687-701; Belle et al., Eur J Haematol 2008, 81: 26-35; Bund et al., Exp Hematol 2007, 35: 920-930; Chen et al., Neoplasia 2008, 10: 977-986; Coleman et al., Int J Cancer 2011, 128: 2114-2124; Dong et al., Cancer Lett 2004, 211: 219-25; Erfurt et al., Int J Cancer 2009, 124: 2341-2346; Flad et al., Proteomics 2006, 6: 364-374; Fleischhauer et al., Cancer Res 1998, 58: 2969-2972; Friedman et al., J Immunol 2004, 172: 3319-3327; Gardyan et al., Int J Cancer, 2015, 136911): 2588-2597; Gomi et al., J Immunol 1999, 163: 4994-5004; Greiner et al., Blood 2005, 106: 938-945; Greiner et al., Blood 2012, 120: 1282-1289; Hardwick et al., Cancer Immun. 2013, 13: 16; Harz et al., J Immunol. 2014, 193(6): 3146-3154; Hernandez et al., Proc Natl Acad Sci USA 2002, 99: 12275-80; Hundemer et al., Exp Hematol 2006, 34: 486-496; Ito et al., Int J Cancer 2000, 88: 633-639; Kao et al., J Exp Med 2001, 194: 1313-1323; Kawahara et al., Oncol Rep 2011, 25: 469-476; Keogh et al., Cancer Res 2000, 60: 3550-3558; Kierstead et al., Br J Cancer 2001, 85: 1738-1745; Kikuchi et al., Int J Cancer 1999, 81: 459-466;Koga et al., Tissue Antigens 2003, 61: 136-145; Li et al., Clin Exp Immunol 2005, 140: 310-319; Li et al., Med oncol., 2014, 31(12): 293; Maccalli et al., Clin Cancer Res 2008, 14: 7292-7303; Machlenkin et al., Cancer Res 2005, 65: 6435-6442; Mahlendorf et al., Cancer Biol. Ther. 2013, 14: 254-261;Maletzki et al., Eur. J. Cancer 2013, 49: 2587-2595; Meier et al., Cancer Immunol Immunother 2005, 54: 219-228; Nonaka et al., Tissue Antigens 2002, 60: 319-327; Sedegah et al., PLos One, 2014, 9(9): e106241; Quintarelli et al., Blood 2011, 117: 3353-3362; Tang et al., Mol Med Rep. 2015, 12(2): 1741-1752; and Tu et al., J Immunother 2012, 35: 235-244; the content of each of which is herein incorporated by reference in their entirety.

In some embodiments, payloads of the conjugates may be TAA or antigenic peptide analogs. An antigenic peptide analog such as a neoantigen analog may be a molecule that is not identical, but retains the biological activity (e.g., immunogenicity) and/or has analogous structural features to a corresponding naturally occurring tumor specific antigen such as neoantigen. TAA and antigenic peptide analogs may be substituted and/or homologous peptides related to a naturally occurring antigenic peptide, such as altered peptide ligands (Kersh and Allen, Essential flexibility in the T-cell recognition of antigen. Nature. 1996, 380: 495-498). Those substitutes and homologs retain similarities to the original peptides and are recognized in a highly similar fashion (e.g., Macdonald et al., T cell allorecognition via molecular mimicry. Immunity. 2009, 31:897-908). The peptide analogs are intended to increase characteristics of naturally occurring antigenic peptides such as resistance against peptide degradation and enhancing the activity of the native epitope to induce cytotoxic T lymphocytes.

In some embodiments, TAA or antigenitc peptide analogs may be biochemically modified as necessary to provide some desired attributes such as improved pharmacological characteristics, while increasing or at least retaining substantially all of the biological activity of the unmodified antigenic peptides to bind the desired MHC molecules and activate the appropriate T cells. Such modifications may also increase the protease resistance, membrane permeability, or half-life without altering, for example, ligand binding.

Accordingly, a TAA or an antigen peptide may be subject to various modifications, such as substitutions, either conservative or non-conservative, where such changes might provide for certain advantages in their use, such as improved MHC molecule binding. By conservative substitutions is meant replacing an amino acid residue with another which is biologically and/or chemically similar, e.g., one hydrophobic residue for another, or one polar residue for another. The substitutions include combinations such as Gly, Ala; Val, Ile, Leu, Met; Asp, Glu; Asn, Gln; Ser, Thr; Lys, Arg; and Phe, Tyr. The effect of single amino acid substitutions may also be probed using D-amino acids. Such modifications may be made using well known peptide synthesis procedures, as described in e.g., Stewart & Young, Solid Phase Peptide Synthesis, (Rockford, Ill., Pierce), 2d Ed. (1984).

The TAA and antigenic peptide may also be modified by extending or decreasing the amino acids of the peptide, such as by the addition or deletion of amino acids.

In one embodiment, an antigenic peptide may include amino acid minics and unnatural amino acids, such as 4-chlorophenylalanine, D- or L-naphylalanine; D- or L-phenylglycine; D- or L-2-thieneylalanine; D- or L-1, -2, 3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoro-methyl)-phenylalanine; D-p-fluorophenylalanine; D- or L-p-biphenyl-phenylalanine; D- or L-p-methoxybiphenylphenylalanine; D- or L-2-indole(alkyl)alanines; and, D- or L-alkylalanines, where the alkyl group can be a substituted or unsubstituted methyl, ethyl, propyl, hexyl, butyl, pentyl, isopropyl, iso-butyl, sec-isotyl, iso-pentyl, or a non-acidic amino acid residues. Aromatic rings of a non-natural amino acid include, e.g., thiazolyl, thiophenyl, pyrazolyl, benzimidazolyl, naphthyl, furanyl, pyrrolyl, and pyridyl aromatic rings. Modified peptides with amino acid mimetics or unnatural amino acid residues may manifest increased stability in vivo.

In addition, an antigenic peptide may be modified by N-terminal acylation, e.g., by alkanoyl (C₁-C₂₀) or thioglycolyl acetylation, and/or C-terminal amidation, e.g., ammonia, methylamine, etc. In some instances these modifications may provide sites for connecting to a linker within the conjugate.

In some embodiments, a mixture of antigenic peptides derived from a single TAA may be used as payloads of the present conjugates. In some instances, the peptide mixture may be a mixture of HLA class I specific epitopes and HLA class II specific epitopes.

In some embodiments, more than one antigenic peptide may be included into a conjugate. The peptides may be selected from a spectrum of different antigens that are associated with a particular cancer. Multiple TAA payloads may enhance the coverage of tumor antigens from a target cancer and therefore enhance the capability of antigen presentation and infiltrate sufficient effector T cells to kill tumor cells. There are several advantages using multiple antigens including i): increasing likelihood of generating a robust immune response against at least some of the antigens; and ii): decreasing the likelihood of a tumor escaping the immune response by immunoediting, because it must downregulate multiple targets. As a non-limiting example, two, three, four, five, six or seven antigens from a list of known HCC specific antigens: alpha-fetoprotein (AFP), glypican-3 (GPC3), NY-ESO-1, SSX-2, melanoma antigen gene-A (MAGE-A), telomerase reverse transcriptase (TERT), and hepatocellular carcinoma-associated antigen-519/targeting protein for Xklp-2 (HCA519/TPX2), may be selected as payloads of a conjugate. Such conjugates may enhance an immune response against HCC tumor cells.

In some aspects, Conjugates comprising antigen payloads may comprise at least two or more neoantigenic peptides. In some embodiments the composition contains at least two distinct peptides. Preferably, the at least two distinct peptides are derived from the same polypeptide (e.g., the same TAA). By distinct polypeptides is meant that the peptide vary by length, amino acid sequence or both.

In some embodiments, payloads of the conjugates of the present invention may comprise between 1 to 20 antigen peptides, for example, 2, 3,4, 5, 6, 7, 8, 9, 10, 11, 12, 13, 14, 15, 16, 17, 18, 19, or 20 different antigen peptides. In other aspects, more than 20 antigen peptides may be included in the conjugates as payloads.

In some embodiments, antigen payloads may be “personalized” tumor antigens from a subject who has a tumor. As used herein, the term “personalized tumor antigens” refers to individual patient specific neoantigens that are encoded by a collective of the individual patient's tumor-specific alternations and mutations. In other aspects, tumor antigen payloads may be “shared” tumor antigens. As used herein, the term “shared tumor antigens” refers to a collective of neoantigens that are commonly presented in a specific type of tumor for example breast tumor.

In accordance with the present invention, for the activation of fully functional cytotoxic T lymphocytes, TAA-derived CD4+ T helper cell epitopes may be induced in a conjugate along with CD8+ T-cell epitopes.

In some embodiments, TAAs may be lipid molecules, polysaccharides, saccharides, nucleic acids, haptens, carbohydrate, or the combinations thereof.

### 2. APC Activation, Maturation and Migration

Antigen presenting cells (APCs), in particular dendritic cells (DCs) are required for presenting a TAA to T cells and activating cancer specific immune responses. Many strategies have been developed to enhance activity of DCs to elicit a specific immune response. Accordingly, payloads of the present conjugates may be any active agents that can increase APCs (i.e. DCs) activity. The active agents may function at any step during the process of dendritic cell maturation, migration, activation and antigen presentation, and/or cytokine production.

In some embodiments, a payload may be an active agent that can promote DCs recruitment, maturation and migration along the lymphatic vessels and into the Lymph Node (LN) (e.g., tumor draining lymph node), therefore, promoting scanning a vast T cell repertoire within the LN.

In some embodiments, a payload may be an agent that can enhance antigen presentation of DCs, i.e. converting antigens into peptide-MHC complexes. The active agent may increase antigen uptake from e.g., death cells of tumors, and efficiently extract peptides from them.

In some embodiments, an active agent may be a chemokine that binds to a chemokine receptor on DCs to regulate DCs. Migration of antigen loaded dendritic cells into lymphatic vessels to lymph node to encounter T cells requires chemokine stimulation and induction of the chemokine receptors (e.g., CCR7). DCs express a panel of inflammatory chemokine receptors including CCR1, CCR2, CCR4, CCR5, CCR6, CCR 8, CCR9, CXCR3, CX3CR, CXCR4 and CCR7, each of which binds to one or more ligands to regulate different aspects of DC maturation, migration, and interaction with naïve T cells in lymph nodes. Some ligands that bind to and activate these receptors include, but are not limited to, CCL3 (MIP1α), CCL5 (RANTES), CCL7 (MCP-3), CCL8 (MCP-2), CCL9 (MRP-2), CCL14 (HCC1), CCL16 (HCC4) which are CCR1 ligands; CCL2 (MCP-1), CCL7 (MCP-3), CCL12 (MCP-5), CCL8 (MCP-2), CCL16 (HCC4) which are CCR2 ligands; CCL17 (TARC), CCL19 (MIP-3β, ELC) which are CCR4 ligands; CCL3 (MIP1α), CCL4 (MIP1β), CCL5 (RANTES), CCL8 (MCP-2), CCL11 (eotaxin), CCL14 (HCC1), CCL16 (HCC4) which are CCR5 ligands; CCL20 (MIP-3α), a ligand of CCR6; CCL1 (TCA3), a ligand of CCR8; CCL25 (TECK), a ligand of CCR9; CXCL9 (Mig), CXCL10 (IP10), CXCL11 (ITAC) which are ligands of CXCR3; CX3C11 (fractalkine), a ligand of CX3CR; CCL12 (SDF-1), a ligand of CXCR4; CCL19 (MIP-3β, ELC), CCL21 (6-Ckine, SLC) which are ligands of CCR7.

For instance, the chemokine receptor CCR7 on DCs, when binding to its ligand CCL19 and CCL21 can regulate the migratory speed of DCs, directing DCs to secondary lymphoid nodes and to elicit an adaptive immune response. (Riol-Blanco et al., The chemokine receptor CCR7 activates in dendritic cells two signaling modules that independently regulate chemotaxis and migratory speed. J Immnuno., 2007, 174(7):4070-80; and Verdijk et al., Maximizing dendritic cell migration in cancer immunotherapy. 2008, Expert Opin Biol Ther., 8(7): 865-874).

In some embodiments, a payload may be a cytokine that can stimulate/regulate the expression both MHC/HLA class I and class II molecules on APCs (i.e. DCs). Interferon-γ (IFN-γ), for example, increases the expression of MHC/HLA class I and MHC/HLA class II molecules, and can induce the expression of MHC/HLA class II molecules on certain cell types that do not normally express them. Interferons also enhance the antigen presenting function of MHC/HLA class I molecules by inducing the expression of key components of the intracellular machinery that enables peptides to be loaded onto the MHC molecules.

Payloads may also be other agents that can stimulate and induce antigen presenting function of other cells for example, γδ T cells. As non-limiting examples, some small molecular weight non-peptide compounds that can stimulate and induce antigen presenting function of γδ T cells may include isopentenyl pyrophosphate (IPP) and others disclosed by Brandes et al (U.S. Pat. No. 8,153,426, which is incorporated herein by reference in its entirety).

It is indicated in many studies that in some tumor cells, antigen presentation is reduced or impaired due to impairment of one or more components of MHC class I/II antigen presenting pathway. For example, mutations which cause a reduced expression of a component, e.g., reduced expression of MHC class I gene due to changes in methylation or chromatin structure, or cause a mutated component that has reduced or no function. Impairments in these components typically affect processing (e.g., proteolysis) of proteins to form peptide epitopes, or transporting peptide to the endoplasmic reticulum, or formation or transport of peptide/MHC molecule (pMHC) complex to the cell surface. As non-limiting examples, components may be MHC class I alpha chain polypeptide, beta2m macroglobulin and TAP.

In certain embodiment, the payload of the conjugate may be a MHC/HLA molecule or a variant thereof that contains sequences to match any known TAA or peptide epitope. Conjugates comprising such molecules may mimic DC derived function to directly activate CD8+ and CD4+ T cells inducing a strong immunogenic response against tumor. The antigen presenting molecules may be MHC/HLA class I or class II molecules.

MHC/HLA class I molecules are cell surface glycoproteins and are heterodimeric and composed of a polymorphic, MHC-encoded, approximately 45 kD α chain, which is non-covalently associated with an approximately 12 kD β-2 microglobulin (β-2m). The extracellular portion of the MHC Class I α chain is divided into three domains, α-1, α-2, and α-3, each approximately 90 amino acids long and encoded on separate exons. The α-3 domain and β-2m are relatively conserved and show amino-acid sequence homology to immunoglobulin constant domains. The polymorphic α-1 and α-2 domains show no significant sequence homology to immunoglobulin constant or variable region. The polymorphic α-1 (approximately 90 amino acids) and α-2 (approximately 92 amino acids) domains are responsible to antigen recognition. The α-2 domain is attached to the less-polymorphic, membrane-proximal α-3 (approximately 92 amino acids) domain which is followed by a conserved transmembrane (25 amino acids) and an intra-cytoplasmic (approximately 30 amino acids) segment.

The classical class I gene family includes the highly polymorphic human class I molecules HLA-A, HLA-B, and HLA-C. -B, and -C genes encode molecules that bind antigenic peptides, and present the peptides to CD8⁺ T cells, thereby initiating a cytotoxic T cell (CTL) response during infection. Extensive allelic polymorphisms are observed in the HLA-A, B and C genes, concentrated primarily among nucleotides that encode residues within the peptide binding grooves of the HLA class I molecules, which determine specificity for the associated peptide ligands.

In some embodiments, payloads may be a polypeptide encoded by any of the known HLA genetic loci, as well as polypeptides encoded by genetic loci not yet discovered so long as these can present antigen to a T cell in a manner effective to activate the T cell receptor. Examples of known HLA class I genetic alleles include: for HLA-A: A*01, A*02, A*03, A*11, A*23, A*24, A*25, A*26, A*28, A*29, A*30, A*31, A*32, A*33, A*34, A*36, A*43, A*66, A*68, A*74 and A*80; for HLA-B: B*07, B*08, B*13, B*14, B*15, B*18, B*27, B*35, B*37, B*38, B*39, B*40, B*41, B*42, B*44, B*45, B*46, B*47, B*48, B*49, B*50, B*51, B*52, B*53, B*54, B*55, B*56, B*57, B*58, B*59, B*67, B*73, B*78, B*81, B*82 and B*83; and for HLA-C: C*01, C*02, C*03, C*04, C*05, C*06, C*07, C*08, C*12, C*14, C*15, C*16, C*17 and C*18.

The polypeptides of HLA class II α and β chain proteins may include polypeptides from genetic loci for HLA-DRA, HLA-DRB1, HLA-DRB3, HLA-DRB4, HLA-DRB5, HLA-DQA, HLA-DQB, HLA-DOA, HLA-DOB, HLA-DMA, HLA-DMB, HLA-DPA and HLA-DPB.

The MHC/HLA polypeptides selected for inclusion in the present conjugates may also include polypeptide variants such as a modified polypeptide.

In some embodiments, conjugates comprising HLA-A, HLA-C, TAP and beta2m polypeptides may be delivered to tumor cells to restore antigen presentation in tumor cells, therefore activate and expand tumor specific cytotoxic T lymphocytes (CTL) to kill tumor cells.

In some aspects, HLA-A, HLA-B and HLA-C, TAP and beta2m payloads of the conjugates may be connected to a targeting moiety through the linker. Such conjugates, in some aspects, may be fused or co-conjugated with one or more TAAs or peptide epitopes. The peptide-MHC molecule (pMHC) complexes may be delivered to a subject directly targeted to tumor cells.

In addition to dendritic cells, accumulating evidence demonstrates that B cells can serve for the antigen-presenting function, beside antibody mediated mechanisms. CD40 Activated antigen-presenting B cells have been shown to efficiently induce both CD4⁺ and CD8⁺ T cells responses in vitro and in vivo. B cell-based vaccines as an alternative to DC-based vaccines for cancer immunotherapy (von Bergwelt-Baildon et al., Human primary and memory cytotoxic T lymphocyte responses are efficiently induced by means of CD40-activated B cells as antigen-presenting cells: potential for clinical application, Blood, 2012, 99:3319-3325). In some embodiments, the conjugate of the present invention may comprise an active agent that can activate B cell antigen presentation.

### 3. T Cell Activation or NK Cell Activation

During a cancer specific immune response, effector T cells (e.g. CD4+ T cells and CD8+ T cells) which are activated by tumor antigen specific APCs can recognize antigen specific tumor cells to kill them. In accordance to the present invention, a payload may an agent that can active effector T cells, or assist T cells in killing tumor cells, or increase the specificity of effector T cells to specific tumor cells.

In some embodiment, the active agent may be an agent that can enhance TAA processing and presentations such as other signals that are provided to T cells by natural antigen presenting cells (APCs). T cell immune responses are mediated by the signals received from APCs. In addition to the interaction between a T cell receptor (TCR) and specific tumor antigen in the form of a peptide/major histocompatibility complex (pMHC) on APCs, co-stimulation between T cells and APCs can amplify antigen-specific T cell responses (Michel, et al., Immunity, 2001, 15(6):935-945). Co-stimulation can be mediated by the interaction between receptors on APCs and their corresponding receptors on T cells. Additionally, cytokines secreted by activated APCs after T cell encounters can stimulate T cell response (Schluns and Lefrancois, Cytokine control of memory T-cell development and survival. Nat. Rev. Immunol., 2003, 3(4):269-79). Accordingly, active agents of the present conjugates may be one or more co-stimulatory agents. In addition to tumor antigens/MHC complexes, such co-stimulatory agents may impact expansion, survival, effector function, and memory of stimulated T cells, the co-stimulatory agents may include but are not limited to antigens, polyclonal T cell receptor activators, co-stimulatory and targeting molecules, and cytokines, which allow for control over the signals provided to T cells by natural APCs. These fully activated signals can be transmitted to the nucleus and result in clonal expansion of T cells, upregulation of activation markers on the cell surface, differentiation into effector cells and induction of cytotoxicity or cytokine secretion.

In some embodiments, the active agent may be a polyclonal T cell receptor activator. As used herein, a polyclonal TCR activator can activate T cells in the absence of specific antigens. Suitable polyclonal T cell activators include the mitogenic lectins concanavalin-A (ConA), phytohemagglutinin (PHA) and pokeweed mitogen (PWM), and antibodies that crosslink the T cell receptor/CD3 complex. Exemplary antibodies that crosslink the T cell receptor include the HIT3a, UCHT1 and OKT3 monoclonal antibodies.

In some embodiments, the active agent may be a co-stimulatory molecule, or any compound that has similar function. Activation and proliferation of T cells are also regulated by both positive and negative signals from costimulatory molecules. One extensively characterized T cell costimulatory pathway is B7-CD28, in which CD80 (B7-1) and CD86 (B7-2) on APCs can interact with stimulatory CD28 receptor and the inhibitory CTLA-4 (CD152) receptor on T cells, respectively. In conjunction with signaling through the T cell receptor, CD28 ligation increases antigen-specific proliferation of T cells, enhances production of cytokines, stimulates differentiation and effector function, and promotes survival of T cells.

In some aspects, a conjugate of the present invention may comprise at least one costimulatory molecule or agent that can stimulate those co-stimulatory effects, as an active agent to be connected to the targeting moiety through the linker. As used herein, the term “co-stimulatory molecule”, in accordance with its meaning in immune T cell activation, refers to a group of immune cell surface receptor/ligands which engage between T cells and APCs and generate a stimulatory signal in T cells which combines with the stimulatory signal in T cells that results from T cell receptor (TCR) recognition of antigen/MHC complex (pMHC) on APCs. Exemplary co-stimulatory molecules, also referred to as “co-stimulators”, include, but are not limited to, CD7, B7-1 (CD80), B7-2 (CD86), 4-1BBL receptor (CD137), 4-1BB ligand (CD137-L), OX40L, inducible co-stimulatory ligand (ICOS-L), intercellular adhesion molecule (ICAM), CD2, CD5, CD9, CD30L, CD40, CD70, CD83, HLA-G, MICA, MICB, HVEM, lymphotoxin beta receptor, 3/TR6, ILT3, ILT4, HVEM, glucocorticoid-induced tumor necrosis factor receptor ligand (GITR-L), an agonist or antibody that binds Toll ligand receptor and a ligand that specifically binds with B7-H3. Other exemplary co-stimulatory molecules that can be used include antibodies that specifically bind with a co-stimulatory molecule present on a T cell, such as, but not limited to, CD27, CD28, 4-IBB, OX40, CD30, CD40, PD-1, ICOS, lymphocyte function-associated antigen-1 (LFA-1), CD2, CD7, LIGHT, NKG2C, B7-H3, and a ligand that specifically binds with CD83. Other suitable costimulatory molecules include, but are not limited to, costimulatory variants and fragments of the natural ligands described above.

As a non-limiting example, a variant may be a soluble form of a co-stimulatory molecule. The soluble form of a co-stimulatory molecule is a fragment of a full length co-stimulatory molecule only containing one or more extracellular domains of the co-stimulatory molecule (e.g., U.S. Pat. No. 8, 268,788). The soluble form of a co-stimulatory molecule derived from an APC retains the ability of the native co-stimulatory molecule to bind to its cognate receptor/ligand on T cells and stimulate T cell activation. A non-limiting example is a soluble form of CD137-L.

In other aspects, the active agent of the conjugate may be a T cell adhesion molecule that can increase the binding association between the antigen-loaded/activated APCs and T cells. Suitable adhesion molecules include, but are not limited to, CD11 a (LFA-1), CD1 1 c, CD49d/29(VLA-4), CD50 (ICAM-2), CD54 (ICAM-1), CD58 (LFA-3) CD102 (ICAM-3) and CD106 (VCAM), and antibodies to their ligands. Other suitable adhesion molecules include antibodies to selectins L, E, and P.

In some embodiments, the active agent of the conjugate may be a cytokine or other immunoregulatory agent. Cytokines may be secreted by activated APCs after T cell encounters and impact expansion, survival, effector function, and memory of stimulated T cells. In some embodiments, at least one cytokine may be connected to the targeting moiety through the linker. Suitable cytokines include, but are not limited to, hematopoietic growth factors, interleukins, interferons, immunoglobulin superfamily molecules, tumor necrosis factor family molecules and chemokines. Preferred cytokines include, but are not limited to, granulocyte macrophage colony stimulating factor (GM-CSF), tumor necrosis factor alpha (TNFα), tumor necrosis factor beta (TNFβ), macrophage colony stimulating factor (M-CSF), interleukin-1 (IL-1), interleukin-2 (IL-2), interleukin-4 (IL-4), interleukin-5 (IL-5), interleukin-6 (IL-6), interleukin-10 (IL-10), interleukin-12 (IL-12), interleukin-15 (IL-15), interleukin-21 (IL-21), interferon alpha (IFNα), interferon beta (IFNβ), interferon gamma (IFNγ), and interferon-gamma inducing factor (IGIF), and variants and fragments thereof.

In some embodiments, TAAs and/or antigenic peptides derived from TAAs, costimulatory factors, T cell adhesion molecules and cytokines secreted by activated APCs may be connected to the targeting moiety through the linker in one conjugate. Alternatively, conjugates comprising each individual agent may be packaged into one particle or a formulation of the present invention.

In certain embodiment, a payload may be a T cell receptor (TCR) or a TCR analog (e.g., engineered CAR) having antigenic specificity for a TAA, e.g., any antigen peptide as discussed above. Mature T cells express a unique αβ TCR that can bind to peptides presented by MHC molecules. Unlike antibodies, TCRs generally have low affinity for ligands, facilitating a rapid scanning of antigen peptide-MHC complexes. Particularly, CDR3 loops of a TCR primarily engage the binding with antigen peptide presented in the MHC groove, while CDR1 and CDR2 loops can contact with the tops of the MHC helices (Garcia and Adams, How the T cell receptor sees antigen-a structural view. Cell. 2005, 122: 333-336; Rudolph et al., How TCRs bind MHCs, peptides, and coreceptors. Annual Review of Immunology. 2006, 24: 419-466).

Tumor specific TCRs may be obtained from spontaneously occurring tumor-specific T cells in patients, such as the melanocyte differentiation antigens MART-1 and gp100, as well as the MAGE antigens and NY-ESO-1, with expression in a broader range of cancers. TCRs may also be isolated from viral infected cells in some viral-associated malignancies. Additionally, TCRs specific to a TAA may also be identified by, for example, allogeneic TCR and transgenic mice expressing human a HLA molecule. Alternatively, recombinant technology can be used to generate TCRs on phage display libraries, which can be used to identify novel high affinity tumor-specific TCRs (Zhao et al., High-affinity TCRs generated by phage display provide CD4+ T cells with the ability to recognize and kill tumor cell lines. J. Immunol. 2007, 179:5845-5854). Isolated TCRs may be used as active agents of the conjugates of the present invention.

In one example, a TCR active agent of the conjugate of the present invention may be a CDR3 region peptide of TCR against a specific TAAs such as WT-1 as disclosed in US patent publication NO. 2014/0315735; the content of which is herein incorporated by reference in its entirety.

In other embodiments, the TCR may be γδ T-cell receptors consisting of a γ chain and a δ chain polypeptide. γδ T-cell receptors may be specialized to bind certain kinds of ligands, including heat-shock proteins and nonpeptide ligands such as mycobacterial lipid antigens. It seems likely that γδ T-cell receptors are not restricted by the ‘classical’ MHC class I and class II molecules. They may bind the free antigen, much as immunoglobulins do, and/or they may bind to peptides or other antigens presented by non-classical MHC-like molecules. These are proteins that resemble MHC class I molecules but are relatively nonpolymorphic.

In accordance with the present invention, a TCR analog may be a chimeric antigen receptor (CAR) that can recognize a specific cell surface tumor antigen independent of MHC/HLA molecules and employs one or more signaling molecules to activate genetically modified T cells for killing, proliferation, and cytokine production. An engineered chimeric antigen receptor (CAR) may be composed of an antibody-derived targeting domain (i.e., an extracellular domain derived from tumor-specific antibody) fused with T-cell signaling domains that, when expressed by a T-cell, endows the T-cell with antigen specificity determined by the targeting domain of the CAR.

The targeting domain of a CAR may be derived from any antibody that specifically recognizes a tumor specific antigen. In some aspects, a single-chain variable fragment (ScFv) of antibodies are used in the extracellular domain of CARs, which are joined through hinge and transmembrane regions to intracellular signaling domains. Tumor-specific antibodies may be generated through immunization of mice. Recombinant techniques can be used to humanize antibodies, or mice expressing human immunoglobulin genes can be used to generate fully human antibodies.

As discussed previously, complete T cell activation is a complex process involving several signals including a primary initiating signal and secondary costimulatory signals. Inclusion of such signals in CARs can enable responses against cancer cells. For example, inclusion of a primary signaling molecule CD3-ζ in CARs can induce T cell activation. Inclusion of the cytoplasmic domain of CD28, CD134 or 4-1BB (CD137) in CARS can lead to increased cytokine production in response to a TAA (e.g., Carpenito et al., Control of large, established tumor xenografts with genetically retargeted human T cells containing CD28 and 4-1BB (CD137) domains. Proc Natl Acad Sci USA. 2009, 106:3360-3365).

CARs specific for a wide range of TAAs have been developed, for example, CD19 specific CAR for leukemia (Kochenderfer et al., adoptive transfer of syngeneic T cells transduced with a chimeric antigen receptor that recognizes murine CD19 can eradicate lymphoma and normal B cells. Blood, 2010, 116: 3875-3886), Chmielewski et al., T cells that target carcinoembryonic antigen eradicate orthotopic pancreatic carcinomas without inducing autoimmune colitis in mice. Gastroenterology. 2012, 143:1095-1107; Westwood et al. Adoptive transfer of T cells modified with a humanized chimeric receptor gene inhibits growth of Lewis-Y-expressing tumors in mice. Proc Natl Acad Sci USA. 2005, 102:19051-19056).

In some embodiment, the active agent of the conjugate may be co-receptors of TCRs such as CD4 and CD8. The payload may be a full length of co-receptors CD4 and CD8, or a domain thereof that can bind to a MHC/HLA molecule. In one example, the payload may be a CD4 immunoglobulin-like domain that can bind to an invariant site of the MHC class II molecule, such as the β2 domain. In another example, the payload may be a CD8 domain that can bind to an invariant site of the MHC class I molecule, such as the α3 domain. CD4 and CD8 co-receptors that bind to MHC class II and I molecules respectively, can markedly increase the sensitivity of a T cell to antigen presented by MHC molecules on APCs.

Conjugates comprising TCRs, CARs or co-receptors, or variants thereof may be used to engineered T cells for adoptive immunotherapy. A detailed discussion of adoptive T cell immunotherapy is described in the following sections.

In some embodiments, the active agent of the conjugate is a CD3-binding agent, such as a peptide or derivative that binds to CD3, a CD3 antibody or a CD3-binding fragment thereof. Activation of cytotoxic T cell may occur via binding of the CD3 antigen as effector antigen on the surface of the cytotoxic T cell by the conjugates of the present invention. CD3 (cluster of differentiation 3) complex, or CD3 antigen, is a T cell co-receoptor that helps to activate T cells. CD3 complex may comprise several chians: CD3D (CD3 delta chain), CD3G (CD3 gamma chain), CD3E (CD3 epsilon chain) and/or CD247 (CD3 zeta chain). The CD3-binding agent, CD3 antibody or the CD3-binding fragment may bind to any epitope on any of the chains.

CD3 antigens are cell-surface proteins and are bound to the membrances of all mature T cells. Conjugates of the present invention comprising CD3 binding agents may bind to and activate T cells in the absence of independent TCR/MHC binding. The activated T cell can then exert a cytotoxic effect on tumor cells. In one embodiment, CD3 antigents do not internalize upon binding of the conjugates.

The CD3 binding agent may be a Fab fragment of a CD3 antibody, a single CDR CD3 antibody, a single chain variable fragment (scFv) of a CD3 antibody, a single-chain antibody mimic that is much smaller than an antibody such as nanofitin® (Affilogic). Non-limiting examples of CD3 antibodies or fragments thereof include, a humanized CD3-specific scFv disclosed by Liddy et al. (Nature Medicine, vol.18(6):980 (2012)), a single-chain anti-CD3 antibody derived from UCHT1 disclosed by Kuo et al. (Protein Engineering, Design & Selection, vol.25(10):561 (2012)), an anti-CD3 scFv comprising an amino acid sequence of SEQ ID No.2 in CA2561826 to Wang et al., an anti-CD3 portion of an anti-CD3&anti-EpCAM bispecific antibody (SEQ ID No.1) disclosed in WO2005061547 to Baeuerle et al., a reshaped Fab antibody against human CD3, a reshaped single-domain antibody against human CD3 or a reshaped scFv against human CD3 disclosed in US20050175606 to Huang et al., anti-CD3 V_{H} disclosed in US20050079170 to Gall et al., any CD3-binding scFv including scFv(UCHT-1)-PE38 disclosed in US20020142000 to Digan et al., the contents of each of which are incorporated herein by reference in their entirety.

Alternatively, the active agent of the conjugate activates other effector cells, such as natural killer cells. In some embodiments, the active agent of the conjguate is a CD16 antibody or a CD16-binding fragment thereof. CD16 is an Fc receptor found on the surface of natural killer cells. Conjugates of the present invention binds to CD16 on natural killer cells and activate natural killer cells. Non-limiting examples of CD16 antibodies or CD16-binding fragment thereof include monoclonal antibody of the IgG1 class against human CD16 antigen disclosed in U.S. Pat. No. 5,643,759 to Pfreundschuh, FV antibody constructs comprising binding sites for a CD16 receptor as disclosed in WO2001011059 to Arndt et al., antibodies exhibiting high affinity for the CD16 receptor disclosed in US20060127392 to de Romeuf et al., the contents of each of which are incorporated herein by reference in their entirety.

In some embodiments, the active agent of the conjuate binds to a universal CAR T cell and activates the CAR T cell. The binding between the active agent and the CAR T cell may occur only in the tumor microenvironment, or is activated by light, heat, radiation, or chemical agents such as but not limited to tetracycline.

In some embodiments, the binding site on the CAR T cell, or the active agent may comprise a masking moiety described herein. The binding of the active agent to the CAR T cell may be inhibited or hindered by the masking moiety. For example, the binding may be sterically hindered by the presence of the masking moiety or may be inhibited by the charge of the masking moiety.

Cleavage of the masking moiety, a conformation change, or a chemical transformation may unmask/activate the binding site on the CAR T cells or the active agent. The masking/unmasking process may be reversible or irreversible.

As a non-limiting example, CAR T cells may be constructed by fusing an anti-fluorescein isothiocyanate (FITC) scFv to a CD3 zeta chain containing the intracellular domain of CD137. The active agent may comprise fluorescein. Therefore, the active agent binds to the CAR T cells and activates T cell cytotoxcity.

### 4. Cytokines, Chemokines and Immunoregulatory Molecules

In addition to cytokines, chemokines and growth factors that involve in APC maturation and migration, and T cell activation, as described previously, an immunoregulatory profile is required to trigger an efficient immune response and balance the immunity in a subject. In certain embodiment, a payload of a conjugate of the present invention may be an immunoregulatory molecule. Conjugates may comprise more than one immunoregulatory molecules as payloads, e.g., two, three, four, five, six, seven or more immunoregulatory molecules.

Examples of suitable immunoregulatory cytokines include, but are not limited to, interferons (e.g., IFNα, IFNβ and IFNγ), interleukins (e.g., IL-1, IL-2, IL-3, IL-4, IL-5, IL-6, IL-7, IL-8, IL-9, IL-10, IL-12 and IL-20), tumor necrosis factors (e.g., TNFα and TNFβ), erythropoietin (EPO), FLT-3 ligand, gIp10, TCA-3, MCP-1, MIF, MIP-1α, MIP-1β, Rantes, macrophage colony stimulating factor (M-CSF), granulocyte colony stimulating factor (G-CSF), and granulocyte-macrophage colony stimulating factor (GM-CSF), as well as functional fragments thereof. The most preferred immunomodulatory cytokine is GM-CSF, such as human GM-CSF, including a functional fragment thereof. An alternatively preferred immunomodulatory cytokine is IL-2 or a functional fragment thereof. Any immunomodulatory chemokine that binds to a chemokine receptor, i.e., a CXC, CC, C, or CX3C chemokine receptor, can be used in the context of the present invention. Examples of chemokines include, but are not limited to, MIP-3α (Lax), MIP-3β, Hcc-1, MPIF-1, MPIF-2, MCP-2, MCP-3, MCP-4, MCP-5, Eotaxin, Tarc, Elc, I309, IL-8, GCP-2 Groα., Gro-β., Nap-2, Ena-78, Ip-10, MIG, I-Tac, SDF-1, and BCA-1 (Blc), as well as functional fragments thereof.

In some embodiments, an immunoregulatory payload may be a T cell growth factor, derivative thereof, or any agent that can stimulate T cell proliferation and/or enhance T cell survival during an immune response, resulting in a more effective immune response and increased memory T cell function. T cell growth factors may include, but are not limited to, interleukin (IL)-2, IL-7, IL-IL-9, IL-12, IL-14, IL-15, IL-16, IL-21 and IL-23. In particular, the active agent may be IL-12 alone, or 2 interleukins in different combinations such as IL-2 and IL-7, IL-2 and IL-15, IL-7 and IL-15, IL-2, IL-7 and IL-15, IL-12 and IL-7, IL-12 and IL-15, or IL-12 and IL2.

In some embodiments, an immunoregulatory payload may be a cytokines that can provide a stimulating environment for T cells differentiation. In the context of CD4+ T cells, Naive CD4⁺ T cells have the capacity to differentiate into either polarized Th1, Th2 or Th0 cells with the capacity to produce type 1 (IFN-γ), type 2 (IL-4) or type 0 (IFN-γ+IL-4) cytokines, respectively.

In some embodiments, a payload of a conjugate of the present invention may be any other immunomodulator that can modulate the activity of the immune system. The “immunomodulator” can be a cytokine, a chemokine or an adjuvant, for example, obtained from any suitable source, such as a mammal, e.g., a human.

The cytokine payload may be a full length of a cytokine or functional variants thereof. As used herein, the term “functional variant” as used herein is synonymous with “biologically equivalent variant, “biologically equivalent derivative,” or “biologically equivalent analog”. A function variant may be a functional portion, fusion, or variant of a cytokine, e.g., is capable of engaging respective receptors and initiating signal transduction. Examples of function variants include cytokines lacking their signal peptides, conservative amino acid substitutions, or amino acid substitution at non-essential regions.

As non-limiting examples, cytokine payloads may be a recombinant interferon (rSIFN-co) with changed spatial configuration disclosed by Wei (PCT patent publication No. WO2014/106459, the content of which is incorporated herein by reference it its entirety).

### 5. Antibodies

In certain embodiments, a payload may be an antibody, a fragment of an antibody or a derivative thereof. Antibodies may be immuno-specific for a tumor cell antigen or against immuno-modulatory factors. An antibody that can recognize a TAA and/or a TAA antigenic peptide may be a monoclonal antibody or a polyclonal antibody. The antibody may be generated by standard hybridoma techniques, phase display and recombinant techniques. In some examples, antibodies may recognize tumor antigens that are overexpressed in tumor cells, or tumor antigens associated with Leukaemias and lymphomas such as cell differentiation (CD) antigens, e.g. CD19, CD20, CD21, CD25 and CD37 in non-hodgkin lymphoma, CD33 in acute myeloid leukemia; CD5 in T cell leukemia, or glycoproteins on the cell surface. In other examples, antibodies may recognize non protein antigens such as glycolipids, e.g., ganglioside, and carbohydrates that are associated with tumors. In other examples, antibodies may recognize any one of TAAs as discussed hereinabove.

Some examples of antibodies that can recognize a specific antigen epitope may include, without limitation, anti-HER2, anti-EGFR as disclosed in U.S. Pat. No.: 9,023,362 and 8,722,362; anti-FcγRIIB as disclosed in U.S. Pat. No. 8,784,808; and antibodies against PSCA (prostate stem cell antigen) as disclosed in U.S. Pat. No. 8,404,817;

In some embodiments, a payload may be an agonist antibody that can manipulate a process of a cancer specific immune response. As non-limiting examples, an agonist antibody may be an antibody specific to 4-1BB (CD137) (e.g., PCT patent publication NO. 2006/088464 to Chen et al.; the content of which is incorporated by reference in its entirety). Stimulation of CD137 by agonistic antibody induces vigorous T-cell proliferation and prevents activation-induced cell death, and induces dendritic and NK cell activation as well.

In other aspects, the active agent of the conjugate may be an agonist antibody that specifically binds to an costimulatory molecule selected from CD28, B7-1 (CD80), B7-2 (CD86), 4-1BB (CD137), 4-1BB ligand (CD137-L), OX40, OX40L, inducible co-stimulatory ligand (ICOS-L), ICOS, intercellular adhesion molecule (ICAM), CD30, CD30L, CD40, CD27, CD70, CD83, HLA-G, MICA, MICB, HVEM, lymphotoxin beta receptor, 3/TR6, ILT3, ILT4, HVEM, GITR, GITR-L, TLR agonist, B7-H3, B7-H3 ligand, CD226, ICOS, LFA-1, CD2, CD7, LIGHT, NKG2D, and DNAM-1.

In other aspects, the active agent of the conjugate may be an antagonist antibody that specifically binds to a coinhibitory molecule selected from CTLA-4, PD-1, PD-L1, PD-L2, TIM-3, LAG-3, BTLA, CD160, C200R, TIGIT, KLRG-1, KIR, 2B4/CD244, VISTA and Ara2R.

In some embodiments, an antibody payload may be a bispecific antibody (bsAb) or multiple specific antibody (msAb) (Weidle et al., Tumor-Antigen-Binding Bispecific Antibodies for Cancer Treatment, Seminars in Oncology, 2014, 41(5): 653-660). As used herein, the term “bispecific antibody” refers to an antibody construct that is capable of redirecting immune effector cells to the tumor microenvironment. Clinical studies of various bsAb constructs have shown impressive results in terms of immune effector cell retargeting, target dependent activation and the induction of anti-tumor responses. Some examples of bispecific antibodies include bispecific antibody against TIM-3 and PD-1 in WO201159877 to Kuchroo et al., the content of which is incorporated by reference in its entirety.

### 6. Cell Surface Antigens

In some embodiments, payloads may be cell surface antigens or fragments thereof. The cell surface antigens may be tumor antigents, which are present by MHC I or MHC II molecules on the surface of tumor cells. Tumor antigens may be tumor specific antigens (TSA), which are present only on tumor cells and not on any other cells, or tumor associated antigens (TAA), which are present on some tumor cells and also some normal cells. Tumor antigens may be cancer testis antigens (CTAs), melanocyte differentiation angiens, mutated proteins, overexpressed proteins, and viral antigens. The cell surface antigens may be shared tumor antigens, or neoantigens. Neoantigens, as used herein, refers to tumor-specific antigens derived from mutated proteins that are present only in the tumor. Neoantigens may be identified with any suitable method known in the art, such as reverse immunology comprising the steps of mutanome screening of a subject using massive parallel sequencing (MPS), computational eptitope prediction, and experimental validation of cancer neoantigens disclosed by Yoshimura et al. in J. of Clinical & Cellular Immunology, vol.6:2 (2015), the contents of which are incorporated herein by reference in their entirety.

The cell surface antigens may be recognized by the immune system of a subject. Conjugtes of the present invention comprising such cell surface antigens and targeting moieties attach to a group of target cells in the subject, turning the cells into antigen-presenting cells (APCs) and allowing the cells to be recognized by the immune system of the subject. The attachement of the conjugates of the present invention to the target cells may be in vivo or ex vivo. The receptors on the target cells that bind to the targeting moieties of the conjugates do not internalize after the attachment.

### 7. Other Immunoactive Agents

In some embodiments, cytotoxic agents may be used as payloads (referring to U.S. Pat. No. 6,572,856) (induce innate immune response to destroy cancer cells). One immunotherapeutic approach involves conjugating cytotoxic agents to monoclonal antibodies (mAbs) specific for a particular cancer cell epitope, therefore treating cancers using tissue specific delivery of anti-cancer agents. The cytotoxic agents may include, but are not limited to maytansinoids, auristatins, calicheamicins, CC-1065, duocarmycins, anthracyclines, and doxorubicin derivatives. In some embodiments, cytotoxic agents may be cytotoxic protein including diphtheria toxin, Pseudomonas exotoxin, or cytotoxic portions or variants thereof.

In some embodiments, the active agent of the conjugate of the present invention may be a complement component (e.g., 21 plasma protein C3b)

In some embodiments, the active agent of the conjugate of the present invention may further include an immunomodulatory adjuvant. The immunomodulatory adjuvants are molecules that can increase the immunogenicity of a TAA or conquer the immune tolerance in the tumor microenvironment. (Sun and Liu, Listeriolysin O as a strong immunogenic molecule for the development of new anti-tumor vaccines. Hum Vaccin Immunother, 2013, 9(5): 1058-1068).

In some embodiments, a payload of a conjugate may be a TLR (toll like receptor) agonist. As used herein, the term “TLR agonist” refers to a compound that acts as an agonist of a TLR. TLR agonists can trigger broad inflammatory responses that elicit rapid innate immune response and promote the activation of the adaptive immune response. Examples of TLR agonists include, but are not limited to, polyinosinic acid (poly I:C), an agonist for TLR3; Cytosine-phosphorothioate-guanine (CpG), an agonist for TLR9; imiquimod, a TLR-7 agonist; resiquimod, a TLR-7/8 agonist; loxoribine, a TLR-7/8 agonist; sialyl-Tn (STn), a carbohydrate associated with the MUCI mucin on a number of human cancer cells and a TLR4 agonist; monophosphoryl lipid A (MPL), a TLR-4 agonist; FSL-1, a TLR-2 agonist; CFA, a TLR2 agonist and Pam3Cys, a TLR-1/2 agonist. In some aspects, a TLR agonist may be a TLR1 agonist, a TLR2 agonist, a TLR 3 agonist, a TLR4 agonist, a TLR5 agonist, a TLR6 agonist, a TLR7 agonist, a TLR8 agonist, a TLR9 agonist, or a TLR 10 agonist. In one example, a TLR agonist may be an agonist disclosed in U.S. Pat. No. 7,993,659, which is incorporated herein by reference in its entirety.)

In some embodiments, a payload of the conjugate of the present invention may be mifamurtide. Mifamurtide, muramyl tripeptide phophatidylethanolamine (MTP-PE), is a synthetic analog of a muramyl dipeptide (MDP). Mifamurtide has a longer half-life than MDP, but has similar pharmacological behaviors. The intracellular pattern recognition molecule NOD2 detects mifamurtide and enhances NF-κB signaling. Therefore, conjugates of the present invention comprising mifamurtide can be recognized by NOD2 and can stimulate the production of IL-1β, IL-6 and TNF-α via the activation of NF-κB signaling in moncytes and macrophages.

### B. Linkers

The conjugates contain one or more linkers attaching the active agents and targeting moieties. The linker, Y, is bound to one or more active agents and a targeting ligand to form a conjugate, wherein the conjugate releases at least one active agent upon delivery to a target cell. The linker can be a C₁-C₁₀ straight chain alkyl, C₁-C₁₀ straight chain 0-alkyl, C₁-C₁₀ straight chain substituted alkyl, C₁-C₁₀ straight chain substituted O-alkyl, C₄-C₁₃ branched chain alkyl, C₄-C₁₃ branched chain O-alkyl, C₂-C₁₂ straight chain alkenyl, C₂-C₁₂ straight chain O-alkenyl, C₃-C₁₂ straight chain substituted alkenyl, C₃-C₁₂ straight chain substituted O-alkenyl, polyethylene glycol, polylactic acid, polyglycolic acid, poly(lactide-co-glycolide), polycarprolactone, polycyanoacrylate, ketone, aryl, heterocyclic, succinic ester, amino acid, aromatic group, ether, crown ether, urea, thiourea, amide, purine, pyrimidine, bypiridine, indole derivative acting as a cross linker, chelator, aldehyde, ketone, bisamine, bis alcohol, heterocyclic ring structure, azirine, disulfide, thioether, hydrazone and combinations thereof. For example, the linker can be a C₃ straight chain alkyl or a ketone. The alkyl chain of the linker can be substituted with one or more substituents or heteroatoms. In some embodiments the linker contains one or more atoms or groups selected from —O—, —C(═O)—, —NR, —O—C(═O)—NR—, —S—, —S—S—. The linker may be selected from dicarboxylate derivatives of succinic acid, glutaric acid or diglycolic acid.

In some embodiments, the alkyl chain of the linker may optionally be interrupted by one or more atoms or groups selected from —O—, —C(═O)—, —NR, —O—C(═O)—NR—, —S—, —S—S—. The linker may be selected from dicarboxylate derivatives of succinic acid, glutaric acid or diglycolic acid.

In some embodiments, the linker may be cleavable and is cleaved to release the active agent. In one embodiment, the linker may be cleaved by an enzyme. As a non-limiting example, the linker may be a polypeptide moiety, e.g. AA in WO2010093395 to Govindan, the content of which is incorporated herein by reference in its entirety; that is cleavable by intracellular peptidase. Govindan teaches AA in the linker may be a di, tri, or tetrapeptide such as Ala-Leu, Leu-Ala-Leu, and Ala-Leu-Ala-Leu. In another example, the cleavable linker may be a branched peptide. The branched peptide linker may comprise two or more amino acid moieties that provide an enzyme cleavage site. Any branched peptide linker disclosed in WO1998019705 to Dubowchik, the content of which is incorporated herein by reference in its entirety, may be used as a linker in the conjugate of the present invention. As another example, the linker may comprise a lysosomally cleavable polypeptide disclosed in U.S. Pat. No. 8,877,901 to Govindan et al., the content of which is incorporated herein by reference in its entirety. As another example, the linker may comprise a protein peptide sequence which is selectively enzymatically cleavable by tumor associated proteases, such as any Y and Z structures disclosed in U.S. Pat. No. 6,214,345 to Firestone et al., the content of which is incorporated herein by reference in its entirety. In some embodiments, the linker may be cleavable by lysozyme.

In one embodiment, the cleaving of the linker is non-enzymatic. Any linker disclosed in US 20110053848 to Cleemann et al., the contents of which are incorporated herein by reference in their entirety, may be used. For example, the linker may be a non-biologically active linker represented by formula (I).

In one embodiment, the linker may be a beta-glucuronide linker disclosed in US 20140031535 to Jeffrey, the contents of which are incorporated herein by reference in their entirety. In another embodiment, the linker may be a self-stabilizing linker such as a succinimide ring, a maleimide ring, a hydrolyzed succinimide ring or a hydrolyzed maleimide ring, disclosed in US20130309256 to Lyon et al., the contents of which are incorporated herein by reference in their entirety. In another embodiment, the linker may be a human serum albumin (HAS) linker disclosed in US 20120003221 to McDonagh et al., the contents of which are incorporated herein by reference in their entirety. In another embodiment, the linker may comprise a fullerene, e.g., C₆₀, as disclosed in US 20040241173 to Wilson et al., the contents of which are incorporated herein by reference in their entirety. In another embodiment, the linker may be a recombinant albumin fused with polycysteine peptide as disclosed in U.S. Pat. No. 8,541,378 to Ahn et al., the contents of which are incorporated herein by reference in their entirety. In another embodiment, the linker comprises a heterocycle ring. For example, the linker may be any heterocyclic 1,3-substituted five- or six-member ring, such as thiazolidine, disclosed in US 20130309257 to Giulio, the content of which is incorporated herein by reference in its entirety.

In some embodiments, the linker may be used with compositions of the invention are well known in the art, and include, e.g., thyroglobulin, albumins such as human serum albumin., tetanus toxoid, polyamino acid residues such as poly L-lysine, poly L-glutamic acid, influenza virus proteins, hepatitis B virus core protein, and the like

In some embodiments, the linker may be a hydrophilic linker as disclosed by Zhao et al. in PCT patent publication NO., WO2014/080251; the content of which is incorporated by reference in its entirety. The hydrophilic linkers may contain phosphinate, sulfonyl, and/or sulfoxide groups to link active agents (payloads) to a cell-targeting moiety.

In other embodiments, the linker promotes cellular internalization. In certain embodiments, the linker promotes cellular internalization. A variety of linkers that can be used with the present compositions and methods are described in WO 2004/010957, US2012/0141509, and US2012/0288512, which are incorporated by reference herein in their entirety.

In some embodiments, the linker of the conjugate may be optional. In this context, the active agent and the targeting moiety of the conjugated are directly connected to each other.

### C. Targeting Moieties

In accordance with the present invention, a conjugate can contain one or more targeting moieties or targeting ligands. For example, the conjugate can include an active agent with multiple targeting moieties each attached via a different linker. The conjugate can have the structure X—Y—Z—Y—X where each X is a targeting moiety that may be the same or different, each Y is a linker that may be the same or different, and Z is the active agent (payload).

Targeting ligands or moieties can be polypeptides (e.g., antibodies), peptides, antibody mimetics, nucleic acids (e.g., aptamers), glycoproteins, small molecules, carbohydrates, lipids, nanoparticles.

One barrier in developing cancer vaccine using tumor specific antigens is the less effective delivery of antigens to the antigen presenting cells (APCs). Increasing delivery of tumor specific antigens can enhance antigen presentation. In one embodiment, a targeting moiety may particularly target a conjugate of the present invention to an immune cell, a tumor cell or a location where an anti-cancer immune response occurs.

In some embodiments, the targeting moiety does not substantially interfere with efficacy of the therapeutic agent in vivo. In some cases, the targeting moiety itself can be an active agent. In other aspects, the targeting moiety may contain adjuvant activity, in addition to targeted binding to a cell of interest.

In some embodiments, the targeting moiety, X, may be a peptide such as a TAA peptide epitope (e.g., an amino acid sequence motif) that can specifically bind to a MHC/HLA protein (HLA class I or class II). Peptide epitopes may be any one discussed above as payloads of the conjugates. In this context, a conjugate may contain two or more the same or different antigen epitopes that are connected through a linker; the antigen epitopes will serve as active agents and targeting moieties.

Peptide antigens can be attached to MHC class I/II molecules by affinity binding within the cytoplasm before they are presented on the cell surface. The affinity of an individual peptide antigen is directly linked to its amino acid sequence and the presence of specific binding motifs in defined positions within the amino acid sequence. Such defined amino acid motifs may be used as targeting moieties.

In some embodiments, the targeting moiety, X, may be other peptides such as somatostatin, octeotide, LHRH (luteinizing hormone releasing hormone), epidermal growth factor receptor (EGFR) binding peptide, aptide or bipodal peptide, RGD-containing peptides, a protein scaffold such as a fibronectin domain, a single domain antibody, a stable scFv, or other homing peptides.

As non-limiting examples, a protein or peptide based targeting moiety may be a protein such as thrombospondin, tumor necrosis factors (TNF), annexin V, an interferon, angiostatin, endostatin, cytokine, transferrin, GM-CSF (granulocyte-macrophage colony-stimulating factor), or growth factors such as vascular endothelial growth factor (VEGF), hepatocyte growth factor (HGF), (platelet-derived growth factor (PDGF), basic fibroblast growth factor (bFGF), and epidermal growth factor (EGF).

In some embodiments, the targeting moiety is an antibody, an antibody fragment, RGD peptide, folic acid or prostate specific membrane antigen (PSMA). In some embodiments, the protein scaffold may be an antibody-derived protein scaffold. Non-limiting examples include single domain antibody (dAbs), nanobody, single-chain variable fragment (scFv), antigen-binding fragment (Fab), Avibody, minibody, CH2D domain, Fcab, and bispecific T-cell engager (BiTE) molecules. In some embodiments, scFv is a stable scFv, wherein the scFv has hyperstable properties. In some embodiments, the nanobody may be derived from the single variable domain (VHH) of camelidae antibody.

In some embodiments, the targeting moiety is a tumor cell binding moiety. For example, it may bind to a somatostatin receptor (SSTR) such as SSTR2 on tumor cells or luteinizing hormone releasing hormone receptor (LHRHR or GNRHR) such as GNRHR1 on tumor cells.

In some embodiments, the tumor cell binding moiety binds to a cell surface protein selected from the group consisting of CD20, carcinoembryonic antigen (CEA), epithelial cell adhesion molecule (EpCAM), and CD19. Non-limiting examples of CD19 binding agents that may be used as a tumor cell binding moiety in the conjugates include any CD19 binding agent disclosed in Dreier et al. (J Immunol., vol.170:4397 (2003)), in Klinger et al. (Blood, vol.119:6226 (2012)), or blinatumomab, a bispecific single-chain antibody targeting CD3 and CD19 antigen disclosed in Topp et al. (J Clin Oncol., vol.29:2493 (2011)). Non-limiting examples of CD20 binding agents include anti-CD20/CD3 T cell-dependent bispecific antibody disclosed in Sun et al. (Sci Transl Med., vol.7:287 (2015)) or anti-CD3×anti-CD20 bispecific antibody disclosed in Gall et al. (Exp Hematol., vol.33(4):452 (2005)). Non-limiting examples of CEA binding agents include CEA/CD3-bispecific T cell-engaging (BiTE) antibody disclosed in Osada et al. (Cancer Immunol Immunother., vol.64(6):677 (2015)). Non-limiting examples of EpCAM binding agents include EpCAM/CD3-bispecific T-cell engaging antibody MT110 disclosed in Cioffi et al. (Clin. Cancer Res., vol.18(2):465 (2012)).

In some embodiments, the targeting moiety is a protein scaffold. The protein scaffold may be a non-antibody-derived protein scaffold, wherein the protein scaffold is based on nonantibody binding proteins. The protein scaffold may be based on engineered Kunitz domains of human serine protease inhibitors (e.g., LAC1-D1), DARPins (designed ankyrin repeat domains), avimers created from multimerized low-density lipoprotein receptor class A (LDLR-A), anticalins derived from lipocalins, knottins constructed from cysteine-rich knottin peptides, affibodies that are based on the Z-domain of staphylococcal protein A, adnectins or monobodies and pronectins based on the 10^{th} or 14^{th} extracellular domain of human fibronectin III, Fynomers derived from SH3 domains of human Fyn tyrosine kinase, or nanofitins (formerly Affitins) derived from the DNA binding protein Sac7d.

In some embodiments, the protein scaffold may be based on a fibronectin domain. In some embodiments, the protein scaffold may be based on fibronectin type III (FN3) repeat protein. In some embodiments, the protein scaffold may be based on a consensus sequence of multiple FN3 domains from human Tenascin-C (hereinafter “Tenascin”). Any protein scaffold based on a fibronectin domain disclosed in U.S. Pat. No. 8,569,227 to Jacobs et al., the content of which is incorporated herein by reference in its entirety; may be used as a targeting moiety of the conjugate of the invention.

In some embodiments, the protein scaffold may be any protein scaffold disclosed in Mintz and Crea, BioProcess, vol.11(2):40-48 (2013), the contents of which are incorporated herein by reference in their entirety. Any of the protein scaffolds disclosed in Tables 2-4 of Mintz and Crea may be used as a targeting moiety of the conjugate of the invention.

In some embodiments, the targeting moiety is an arginylglycylaspartic acid (RGD) peptide, a tripeptide composed of L-arginine, glucine and L-aspartic acid, which is a common cell targeting element for cellular attachment via integrins.

In some embodiments, a targeting moiety may be an antibody that specifically binds to a TAA and/or an antigenic peptide (epitope). As one skilled in the art can envision, an antibody fragment (e.g., an Fc fragment of an antibody) may be used for the same purpose.

In addition to tumor cells specific antigen or antigen epitopes, antibodies may be specific to a ubiquitous antigenic site on various cancers. Many studies have revealed that cancer cells share certain common characteristics. Many types of human cancer cells are characterized by substantial abnormalities in the glycosylation patterns of their cell-surface proteins and lipids (e.g., Hakomori et. al., 1996, Cancer Res. 56:5309-18; and Springer et al., 1997, J Mol Med 75:594-602). These differences have led to the identification of antigenic determinants on cancer cells. Natural IgM antibodies to these epitopes are present in the circulation and can be used as a targeting moiety of a conjugate of the present invention.

As non-limiting examples, the antibody targeting moiety may be connected to one or more components of the complement system (or other cytotoxic agents) to induce complement mediated tumor cell lysis. In this context, a conjugate may have a formula of (one or more cytotoxic agents)-linker-mAb.

In some embodiments, the targeting moiety is an antibody mimetic such as a monobody, e.g., an ADNECTIN™ (Bristol-Myers Squibb, New York, N.Y.), an Affibody® (Affibody AB, Stockholm, Sweden), Affilin, nanofitin (affitin, such as those described in WO 2012/085861, an Anticalin™, an avimers (avidity multimers), a DARPin™, a Fynomer™, Centyrin™, and a Kunitz domain peptide. In certain cases, such mimetics are artificial peptides or proteins with a molar mass of about 3 to 20 kDa. Nucleic acids and small molecules may be antibody mimetic.

In some embodiments, the targeting moiety X may be an aptide or bipodal peptide. X may be any D-Aptamer-Like Peptide (D-Aptide) or retro-inverso Aptide which specifically binds to a target comprising: (a) a structure stabilizing region comprising parallel, antiparallel or parallel and antiparallel D-amino acid strands with interstrand noncovalent bonds; and (b) a target binding region I and a target binding region II comprising randomly selected n and m D-amino acids, respectively, and coupled to both ends of the structure stabilizing region, as disclosed in US Pat. Application No. 20140296479 to Jon et al., the content of which is incorporated herein by reference in its entirety. X may be any bipodal peptide binder (BPB) comprising a structure stabilizing region of parallel or antiparallel amino acid strands or a combination of these strands to induce interstrand non-covalent bonds, and target binding regions I and II, each binding to each of both termini of the structure stabilizing region, as disclosed in US Pat. Application No. 20120321697 to Jon et al., the content of which is incorporated herein by reference in its entirety. X may be an intracellular targeting bipodal-peptide binder specifically binding to an intracellular target molecule, comprising: (a) a structure-stabilizing region comprising a parallel amino acid strand, an antiparallel amino acid strand or parallel and antiparallel amino acid strands to induce interstrand non-covalent bonds; (b) target binding regions I and II each binding to each of both termini of the structure-stabilizing region, wherein the number of amino acid residues of the target binding region I is n and the number of amino acid residues of the target binding region II is m; and (c) a cell-penetrating peptide (CPP) linked to the structure-stabilizing region, the target binding region I or the target binding region II, as disclosed in US Pat. Application No. 20120309934 to Jon et al., the content of which is incorporated herein by reference in its entirety. X may be any bipodal peptide binder comprising a β-hairpin motif or a leucine-zipper motif as a structure stabilizing region comprising two parallel amino acid strands or two antiparallel amino acid strands, and a target binding region I linked to one terminus of the first of the strands of the structure stabilizing region, and a target binding region II linked to the terminus of the second of the strands of the structure stabilizing region, as disclosed in US Pat. Application No. 20110152500 to Jon et al., the content of which is incorporated herein by reference in its entirety. X may be any bipodal peptide binder targeting KPI as disclosed in WO2014017743 to Jon et al, any bipodal peptide binder targeting cytokine as disclosed in WO2011132939 to Jon et al., any bipodal peptide binder targeting transcription factor as disclosed in WO201132941 to Jon et al., any bipodal peptide binder targeting G protein-coupled receptor as disclosed in WO2011132938 to Jon et al., any bipodal peptide binder targeting receptor tyrosine kinase as disclosed in WO2011132940 to Jon et al., the content of each of which is incorporated herein by reference in their entirety. X may also be bipodal peptide binders targeting cluster differentiation (CD7) or an ion channel.

In some embodiments, the targeting moiety is a stabilized peptide. Intramolecular crosslinkers are used to maintain the peptide in the desired configuration, for example using disulfide bonds, amide bonds, or carbon-carbon bonds to link amino acid side chains. Such peptides which are conformationally stabilized by means of intramolecular cross-linkers are sometimes referred to as “stapled” peptides. The cross-linkers connect at least two amino acids of the peptide. The cross-linkers may comprise at least 5, 6, 7, 8, 9, 10, 11, or 12 consecutive carbon-carbon bonds. The cross-linkers may comprise at least 5, 6, 7, 8, 9, 10, 11, or 12 carbon atoms. Stapled peptides may penetrate cell membranes and bind to an intracellular receptor.

In one non-limiting example, the stapled peptide is a cross-linked alpha-helical polypeptide comprising a crosslinker wherein a hydrogen atom attached to an α-carbon atom of an amino acid of the peptide is replaced with a substituent of formula R—, wherein R— is alkyl, alkenyl, alkynyl, arylalkyl, cycloalkylalkyl, heteroalkyl, or heterocycloalkyl, unsubstituted or substituted with halo-, as disclosed in US 20140323701 to Nash et al., the contents of which are incorporated herein by reference in their entirety. In another example, the stapled peptides have improved in vivo half life such as any stapled peptide disclosed in US 20100298201 to Nash et al., the contents of which are incorporated herein by reference in their entirety. In another example, the tumor cell binding moiety may be any stapled peptide disclosed in U.S. Pat. No. 9,175,045 to Nash et al., the contents of which are incorporated herein by reference in their entirety, wherein the stapled peptide possesses reduced affinity to serum proteins while still remaining sufficient affinity to cell membranes. In another example, the cross-linker of the stapled peptide links the α-positions of at least two amino acids, such as any stapled peptide disclosed in U.S. Pat. No. 9,175,047 to Nash et al., the contents of which are incorporated herein by reference in their entirety. In another example, the tumor cell binding moiety comprise any stapled peptide disclosed in U.S. Pat. No. 8,927,500 to Guerlavais et al., the contents of which are incorporated herein by reference in their entirety, wherein the stapled peptide has homology to p53 protein and can bind to the MDM2 and/or MDMX proteins. In another example, the stapled peptide generates a reduced antibody response. Any stapled peptide disclosed in U.S. Pat. No. 8,808,694 to Nash et al., the contents of which are incorporated herein by reference in their entirety, may be used as a tumor cell binding moiety. In another example, the stapled peptide may be any polypeptide with optimized protease stability disclosed in US 20110223149 to Nash et al., the contents of which are incorporated herein by reference in their entirety.

In some embodiments, the targeting moiety is a nanofitin® (Affilogic). Nanofitin, as used as herein, refers to a single-chain antibody mimic that are much smaller than antibodies. Nanofitins are small and stable, lack disulfide bridges, and can be produced at high levels. The molecular weight of nanofitins are below 10 KDa, preferably around 7 KDa. Because of their small size and short half-life, nanofitins may both accumulate specifically at the site of the tumor and be cleared from the serum rapidly, therefore reducing off-target toxicity compared to long lasting antibodies. Conjugates comprise nanofitins may deliver an active agent deeper into a tumor. Nanofitins may bind intracellular targets and affect intracellular protein-protein interaction.

In certain embodiments, the targeting moiety may be a bispecific T-cell engagers, an aptamer such as RNA, DNA or an artificial nucleic acid; a small molecule; a carbohydrate such as mannose, galactose or arabinose; a lipid, a vitamin such as ascorbic acid, niacin, pantothenic acid, carnitine, inositol, pyridoxal, lipoic acid, folic acid (folate), riboflavin, biotin, vitamin B12, vitamin A, E, and K.

In some embodiments, the targeting moiety may comprise a nucleic acid targeting moiety. In general, a nucleic acid targeting moiety is any nucleic acid that binds to an organ, tissue, cell, or a component associated therewith such as extracellular matrix component, and intracellular compartment. In some embodiments, the targeting moiety may be an aptamer, which is generally an oligonucleotide (e.g., DNA, RNA, or an analog or derivative thereof) that binds to a particular target, such as a polypeptide. In one embodiment, the targeting moiety may be an aptamer that targets to an immune cell (e.g., dendritic cells). Aptamers may be generated from libraries of single-stranded nucleic acids against different molecules via CELL-SELEX method in which whole living cells (e.g., dendritic cells) are used as targets for the aptamers (Ganji et al., Aptamers: new arrows to target dendritic cells, J Drug Target. 2015, 7: 1-12).

In some embodiments, the targeting moiety may be a non-immunoreactive ligand. For example, the non-immunoreactive ligand may be insulin, insulin-like growth factors I and II, lectins, apoprotein from low density lipoprotein, etc. as disclosed in US 20140031535 to Jeffrey, the content of which is incorporated herein by reference in its entirety. Any protein or peptide comprising a lectin disclosed in WO2013181454 to Radin, the content of which is incorporated herein by reference in its entirety, may be used as a targeting moiety.

In some embodiments, targeting moieties may be Lymph Node-targeting nanoparticle (NP)-conjugates (Jeanbart et al., Enhancing efficacy of anticancer vaccines by targeted delivery to tumor-draining lymph nodes. Cancer Immunol Res., 2014, 2(5): 436-437; the content of which is incorporated by reference in its entirety.

In some embodiments, the conjugate may have a terminal half-life of longer than about 72 hours and a targeting moiety may be selected from Table 1 or 2 of US 20130165389 to Schellenberger et al., the contents of which are incorporated herein by reference in their entirety. The targeting moiety may be an antibody targeting delta-like protein 3 (DLL3) in disease tissues such as lung cancer, pancreatic cancer, skin cancer, etc., as disclosed in WO2014125273 to Hudson, the contents of which are incorporated herein by reference in their entirety. The targeting moiety may also any targeting moiety in WO2007137170 to Smith, the contents of which are incorporated herein by reference in their entirety. The targeting moiety binds to glypican-3 (GPC-3) and directs the conjugate to cells expressing GPC-3, such as hepatocellular carcinoma cells.

In some embodiments, the targeting moiety may be a modified viral surface protein or fragments thereof.

In some embodiments, the targeting moiety may be an antigen recognition domain/sequence of TCR molecules. The nature of antigen recognition of such moieties will bind to an antigen-MHC molecule complex on the surface of cells, therefore deliver an active payload linked to the targeting moieties through a linker in the conjugate to the tumor cells.

In some embodiments, targeting moieties may be derived from the binding domains of the MHC class I and II molecules, for example, the α3 domain of the α chain of the MHC class I molecule. The α3 domain in the MHC class I molecule can specifically bind to CD8 on T cells, and the binding between CD8 and the α3 domain may deliver tumor antigen payloads near to the surface of T cells and activate TCR to bind the tumor antigens. In another example, the targeting moiety may be the (32 domain of the MHC class II molecules.

In some embodiments, the targeting moiety may be a cell binding element such as a ligand which binds to a cell surface receptor. In specific embodiments, the cell binding element may be selected from the group consisting of a Fc fragment, a toxin cell binding domain, a cytokine, a chemokine, a small peptide and an antibody. In some examples, the cytokines, chomekines and other immunomodulatory molecules are ligands of cell receptors on certain types of immune cells such as APCs (e.g., DCs), T cells, B cells, NK cells and macrophages.

In some embodiments, targeting moieties may be used to deliver antigens to APCs (Frenz et al., Antigen presenting cell selective drug delivery by glycan-decorated nanocarriers. Eur J Pharm Biopharm, 2015, Feb. 19, pii: S0939-6411), such as DEC-205 antibody as targeting moieties for targeted delivery of antigens to APCs.

In some embodiments, targeting moieties may be a single-chain antibody mimic that are much smaller than antibodies such as nanofitin® (Affilogic) disclosed in copending U.S. Application No. 62/308,908, or peptides which are conformationally stabilized by means of intramolecular cross-linkers referred to as “stapled” peptides disclosed in copending U.S. Application No. 62/291,212, the contents of each of which are incorporated herein by reference in their entirety.

### Masked Targeting Moiety Complex

In some embodiments, the targeting moiety may be a targeting moiety complex comprising a target binding moiety (TBM) and a masking moiety (MM). In some embodiments, MM may be attached to TBM directly, via a non-cleavable moiety, or via a cleavable moiety (CM). In some other embodiments, MM is bound to the payload or the linker of the conjugate directly, via a non-cleavable moiety, or via a cleavable moiety (CM).

TBM may be any targeting moiety discussed above including small molecules, peptides or derivatives, an antibody or a fragment thereof. In some embodiments, TBM may be a peptide comprising between 5 to 50 amino acids, between 10 to 40 amino acids, or between 20 to 30 amino acids. In some embodiments, TBM may be small molecules.

The binding of TBM to its target is inhibited or hindered by MM. For example, the binding may be sterically hindered by the presence of MM or may be inhibited by the charge of MM. Leaving of MM upon cleavage of CM, a conformation change, or a chemical transformation may unmask TBM. The masking/unmasking process may be reversible or irreversible.

In one example wherein TBM is attached to MM with a CM, TBM might be less accessible to its target when CM is uncleaved. Upon cleavage of CM, MM no longer interferes with the binding of the targeting moiety to its target, thereby activating the conjugates of the present invention. The cleavable moiety prevents binding of the conjugates of the present invention at nontreatment sites. Such conjugates can further provide improved biodistribution characteristics.

MM may be selected from a plurality of polypeptides based on its ability to inhibit binding of the TBM to the target in an uncleaved state and allow binding of the TBM to the target in a cleaved state.

CM may locate between TBM and MM in the targeting moiety complex, or may locate within MM. CM may be cleaved by an enzyme such as protease. CM may comprise a peptide that may be a substrate for an enzyme selected from the group consisting of MMP1, MMP2, MMP3, MMP8, MMP9, MMP14, plasmin, PSA, PSMA, CATHEPSIN D, CATHEPSIN K, CATHEPSIN S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, and TACE. For example, CM may comprise a protease substrate such as a plasmin substrate, a caspase substrate or a matrix metalloprotease (MMP) substrate (e.g., a substrate of MMP-1, MMP-2, MMP-9, or MMP-14). Alternatively, CM may be cleaved by a reducing agent capable of reducing a disulfide bond between a cysteine-cysteine pair. CM may comprise a cysteine-cysteine pair capable of forming a reducible disulfide bond. Reducing agents of particular interest include cellular reducing agents such as proteins or other agents that are capable of reducing a disulfide bond under physiological conditions, e.g., glutathione, thioredoxin, NADPH, flavins, and ascorbate.

In one example, the targeting moiety complex may be any activatable binding polypeptides (ABPs) disclosed in U.S. Pat. No. 9,169,321 to Daugherty et al. (CytomX), the contents of which are incorporated herein by reference in their entirety. For example, the targeting moiety complex may be an enzyme activatable binding polypeptide (ABP) that binds CTLA-4, VEGF, or VCAM-1. In other examples, the the targeting moiety complex may be an activatable binding polypeptide (ABP) that binds epidermal growth factor disclosed in U.S. Pat. No. 9,120,853 to Lowman et al., an ABP that binds Jagged 1 or Jagged 2 disclosed in U.S. Pat. No. 9,127,053 to West et al., activatable anti-CD3 antibodies disclosed in WO2016014974 to Irving et al., activatable antibodies that bind to interleukin-6 receptor (IL6R) disclosed in WO2014052462 to West et al., activatable proproteins disclosed in US20150203559 to Stagliano et al., any modified antibody or activatable antibody disclosed in US20140024810 to Stagliano et al., WO2015089283 to Desnoyers et al., WO2015066279 to Lowman et al., WO2015048329 to Moore et al., US20150079088 to Lowman et al., WO2014197612 to Konradi et al., US20140023664 to Lowman et al., the contents of each of which are incorporated herein by reference in their entirety.

In some embodiments, the targeting moiety may be a targeting moiety complex comprising a target binding moiety (TBM) and a photocleavable moiety. The binding of TBM to its target is reversibly inhibite by the photocleavable moiety. TBM may be any targeting moiety discussed above including small molecules, peptides or derivatives, an antibody or a fragment thereof. A “photocleavable moiety” means any agent attached to the antibody which can be removed on exposure to electromagnetic energy such as light energy of any desired vaπety whether visible, UV, X-ray or the like (e g microwave). The photocleavable moiety may be a reagent which couples to hydroxy or amino residues present in TBM. Thus phosgene, diphosgene, DCCI or the like may be used to generate photocleavable esters, amides, carbonates and the like from a wide range of alcohols. For example, substituted arylalkanols are employed, particularly nitorphenyl methyl alcohol, 1-nitrophenylethan-1-ol and substituted analogues. The photocleavable moiety may be located at or about the binding site of TBM.

In one example, the targeting moiety complex may comprise any photocleavable moiety disclosed in WO1996034892 to Self et al., the contents of which are incorporated herein by reference in their entirety. TBM may be an antibody component that retain the active site and bind to a tumor cell marker. TBM may also be any antibody component made against suitable cells such as T-cells, cytotoxic T-cell clones, cytotoxic T-cells and activated peripheral blood lymphocytes, CD3+ lymphocytes, CD 16+ lymphocytes, Fc gamma R1 11, the low affinity Fc gamma receptor for polymorphonuclear leucocytes, macrophages and large granular lymphocytes, B-lymphocyte markers, myeloid cells, T Lymphocyte CD2, CD3, CD4, CD8, dengue virus, lymphokine activated killer (LAK) cells, NK cells or monocytes. TBM may be a monoclonal antibody anti-CD-3 OKT3 against T-cells, or a monoclonal antibody that binds to tumor antigen carcinoembrionic antigen (CEA).

In certain embodiments, the targeting moiety or moieties of the conjugate are present at a predetermined molar weight percentage from about 1% to about 10%, or about 10% to about 20%, or about 20% to about 30%, or about 30% to about 40%, or about 40% to about 50%, or about 50% to about 60%, or about 60% to about 70%, or about 70% to about 80%, or about 80% to about 90%, or about 90% to about 99% such that the sum of the molar weight percentages of the components of the conjugate is 100%. The amount of targeting moieties of the conjugate may also be expressed in terms of proportion to the active agent(s), for example, in a ratio of ligand to active agent of about 10:1, 9:1, 8:1, 7:1, 6:1, 5:1, 4:1, 3:1, 2:1, 1:1, 1:2, 1:3, 1:4; 1:5, 1:6, 1:7, 1:8, 1:9, or 1:10.

### D. Pharmacokinetic Modulating Unit

The conjugates of the present invention may further comprise at least one external linker connected to a reacting group that reacts with a functional group on a protein or an engineered protein or derivatives/analogs/mimics thereof, or comprise at least one external linker connected to a pharmacokinetic modulating unit. The external linkers connecting the conjugates and the reacting group or the pharmacokinetic modulating units may be cleavable linkers that allow release of the conjugates. Hence, the conjugates may be separated from the protein or pharmacokinetic modulating units as needed.

In some embodiments, the conjugates comprise at least one reacting group that reacts with a functional group on a protein or an engineered protein or derivatives/analogs/mimics thereof. The reaction between the reacting group and the functional group may happen in vivo after administration or is performed prior to administration. The protein may be a naturally occurring protein such as a serum or plasma protein, or a fragment thereof. Particular examples include thyroxine-binding protein, transthyretin, α1-acid glycoprotein (AAG), transferrin, fibrinogen, albumin, an immunoglobulin, α-2-macroglobulin, a lipoprotein, or fragments thereof. The reaction between the reacting group and the functional group may be reversible.

In one example, the functional group is on human serum albumin (HSA or albumin) or its derivative/analog/mimic. Albumin is the most abundant plasma protein (35-50 g/L in human serum) with a molecular weight of 66.5 KDa and an effective diameter of 7.2 nm (Kratz, J. of Controlled Release, vol.132:171, (2008), the contents of which are incorporated herein by reference in their entirety). Albumin has a half-life of about 19 days. Albumin preferentially accumulates in malignant and inflamed tissues due to a leaky capillary and an absent or defective lymphatic drainage system. Albumin accumulates in tumors such as solid tumors also because albumin is a major energy and nutrition source for turmor growth. The function group may be the cysteine-34 position of albumin that has an accessible free thiol group. Reacting groups that react with a functional group on albumin or it derivative/analog/mimic may be selected from a disulfide group, a vinylcarbonyl group, a vinyl acetylene group, an aziridine group, an acetylene group or any of the following groups:

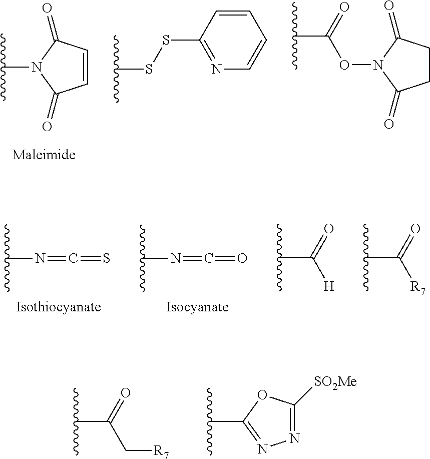

where R⁷ is Cl, Br, F, mesylate, tosylate, O-(4-nitrophenyl), O-pentafluorophenyl, and wherein optionally the activated disulfide group, the vinylcarbonyl group, the vinyl acetylene group, the aziridine group, and the acetylene group may be substituted. The reacting group may also be any protein-binding moiety disclosed in U.S. Pat. No. 9,216,228 to Kratz et al., the contents of which are incorporated herein by reference in their entirety, selected from the group consisting of a maleinimide group, a halogenacetamide group, a halogenacetate group, a pyridylthio group, a vinylcarbonyl group, an aziridine group, a disulfide group, a substituted or unsubstituted acetylene group, and a hydroxysuccinimide ester group. In some cases, the reacting group is a disulfide group. The disulfide group undergoes an exchange with a thiol group on a protein or an engineered protein or a polymer or derivatives/analogs/mimics thereof, such as albumin, to form a disulfide between the conjugate and the protein or an engineered protein or a polymer or derivatives/analogs/mimics thereof.

In another example, the functional group is on transthyretin or its derivative/analog/mimic. Transthyretin is a 55 KDa serum protein that has an in vivo half-life of around 48 h. Reacting groups that react with a functional group on transthyretin or it derivative/analog/mimic may be selected from AG10 (structure shown below) or its derivative disclosed by Penchala et al. in Nature Chemical Biology, vol.11:793, (2015) or formula (I), (II), (III) or (IV) (structures shown below) disclosed in U.S. Pat. No. 5,714,142 to Blaney et al., the contents of each of which are incorporated herein by reference in their entirety. Any transthyretin-selective ligand disclosed on pages 5-8 of Blaney et al. or their derivatives may be used as a reacting group, such as but not limited to, tetraiodothyroacetic acid, 2,4,6-triiodophenol, flufenamic acid, diflunisal, milrinone, EMD 21388.

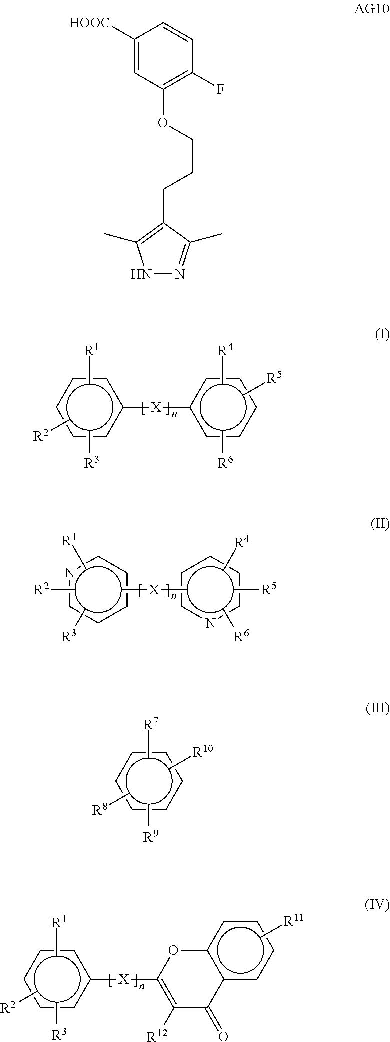

In some cases, the reacting group may be any protein binding moiety may be any protein binding moiety disclosed in U.S. Pat. No. 9,216,228 to Kratz, the contents of which are incorporated herein by reference in their entirety, such as a maleimide group, a halogenacetamide group, a halogenacetate group, a pyridylthio group, a vinylcarbonyl group, an aziridin group, a disulfide group, a substituted or unsubstituted acetylene group, and a hydroxysuccinimide ester group.

In some embodiments, the conjugates comprise at least one pharamacokinetic modulating unit. The pharmacokinetic modulating unit may be a natural or synthetic protein or fragment thereof. For example, it may be a serum protein such as thyroxine-binding protein, transthyretin, α1-acid glycoprotein (AAG), transferrin, fibrinogen, albumin, an immunoglobulin, α-2-macroglobulin, a lipoprotein, or fragments thereof. The pharmacokinetic modulating unit may also be a natural or synthetic polymer, such as polysialic acid unit, a hydroxyethyl starch (HES) unit, or a polyethylene glycol (PEG) unit. Further, the pharmacokinetic modulating unit may be a particle, such as dendrimers, inorganic nanoparticles, organic nanoparticles, and liposomes.

The pharmacokinetic modulating unit or pharmacokinetic modulating units have a total molecular weight of at least about 10 KDa, at least about 20 KDa, at least about 30 KDa, at least about 40 KDa or at least about 50 KDa. Generally, the pharmacokinetic modulating unit or pharmacokinetic modulating units have a total molecular weight between about 10 KDa and about 70 KDa. Preferably, the pharmacokinetic modulating unit or pharmacokinetic modulating units have a total molecular weight between about 30 KDa and about 70 KDa, between about 40 KDa and about 70 KDa, between about 50 KDa and about 70 KDa, between about 60 KDa and about 70 KDa.

### II. Particles and Nanoparticles

Particles comprising one or more conjugates can be polymeric particles, lipid particles, solid lipid particles, solid lipid nanoparticles, solid nanoparticles, inorganic particles, or combinations thereof (e.g., lipid stabilized polymeric particles). In some embodiments, the conjugates are substantially encapsulated or particularly encapsulated in the particles. In some embodiments, the conjugates are disposed on the surface of the particles. The conjugates may be attached to the surface of the particles with covalent bonds, or non-covalent interactions. In some embodiments, the conjugates of the present invention self-assemble into a particle.

As used herein, the term “encapsulate” means to enclose, surround or encase. As it relates to the formulation of the conjugates of the invention, encapsulation may be substantial, complete or partial. The term “substantially encapsulated” means that at least greater than 50, 60, 70, 80, 85, 90, 95, 96, 97, 98, 99, 99.9, 99.99 or greater than 99.999% of conjugate of the invention may be enclosed, surrounded or encased within the particle. “Partially encapsulation” means that less than 10, 10, 20, 30, 40 50 or less of the conjugate of the invention may be enclosed, surrounded or encased within the particle. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the invention are encapsulated in the particle. Encapsulation may be determined by any known method. In some embodiments, the particles are polymeric particles or contain a polymeric matrix. The particles can contain any of the polymers described herein or derivatives or copolymers thereof. The particles will generally contain one or more biocompatible polymers. The polymers can be biodegradable polymers. The polymers can be hydrophobic polymers, hydrophilic polymers, or amphiphilic polymers. In some embodiments, the particles contain one or more polymers having an additional targeting moiety attached thereto. In some embodiments, the particles are inorganic particles, such as but not limited to, gold nanoparticles and iron oxide nanoparticles.

The size of the particles can be adjusted for the intended application. The particles can be nanoparticles or microparticles. The particle can have a diameter of about 10 nm to about 10 microns, about 10 nm to about 1 micron, about 10 nm to about 500 nm, about 20 nm to about 500 nm, or about 25 nm to about 250 nm. In some embodiments the particle is a nanoparticle having a diameter from about 25 nm to about 250 nm. In some embodiments, the particle is a nanoparticle having a diameter from about 50 nm to about 150 nm. In some embodiments, the particle is a nanoparticle having a diameter from about 70 nm to about 130 nm. In some embodiments, the particle is a nanoparticle having a diameter of about 100 nm. It is understood by those in the art that a plurality of particles will have a range of sizes and the diameter is understood to be the median diameter of the particle size distribution. Polydispersity index (PDI) of the particles may be ≤about 0.5, ≤about 0.2, or ≤about 0.1. Drug loading may be ≥about 1%, ≥about 5%, ≥about 10%, or ≥out 20%. Drug loading, as used herein, refers to the weight ratio of the conjugates of the invention and depends on maximum tolerated dose (MTD) of free drug conjugate. Particle ζ-potential (in 1/10^{th} PBS) may be ≤0 mV or from about −10 to 0 mV. Drug released in vitro from the particle at 2 h may be less than about 60%, less than about 40%, or less than about 20%. Regarding pharmacokinetics, plasma area under the curve (AUC) in a plot of concentration of drug in blood plasma against time may be at least 2 fold greater than free drug conjugate, at least 4 fold greater than free drug conjugate, at least 5 fold greater than free drug conjugate, at least 8 fold greater than free drug conjugate, or at least 10 fold greater than free drug conjugate. Tumor PK/PD of the particle may be at least 5 fold greater than free drug conjugate, at least 8 fold greater than free drug conjugate, at least 10 fold greater than free drug conjugate, or at least 15 fold greater than free drug conjugate. The ratio of Cₘₐₓ of the particle to Cₘₐₓ of free drug conjugate may be at least about 2, at least about 4, at least about 5, or at least about 10. Cₘₐₓ, as used herein, refers to the maximum or peak serum concentration that a drug achieves in a specified compartment or test area of the body after the drug has been administrated and prior to the administration of a second dose. The ratio of MTD of a particle to MTD of free drug conjugate may be at least about 0.5, at least about 1, at least about 2, or at least about 5. Efficacy in tumor models, e.g., TGI %, of a particle is better than free drug conjugate. Toxicity of a particle is lower than free drug conjugate.

In various embodiments, a particle may be a nanoparticle, i.e., the particle has a characteristic dimension of less than about 1 micrometer, where the characteristic dimension of a particle is the diameter of a perfect sphere having the same volume as the particle. The plurality of particles can be characterized by an average diameter (e.g., the average diameter for the plurality of particles). In some embodiments, the diameter of the particles may have a Gaussian-type distribution. In some embodiments, the plurality of particles have an average diameter of less than about 300 nm, less than about 250 nm, less than about 200 nm, less than about 150 nm, less than about 100 nm, less than about 50 nm, less than about 30 nm, less than about 10 nm, less than about 3 nm, or less than about 1 nm. In some embodiments, the particles have an average diameter of at least about 5 nm, at least about 10 nm, at least about 30 nm, at least about 50 nm, at least about 100 nm, at least about 150 nm, or greater. In certain embodiments, the plurality of the particles have an average diameter of about 10 nm, about 25 nm, about 50 nm, about 100 nm, about 150 nm, about 200 nm, about 250 nm, about 300 nm, about 500 nm, or the like. In some embodiments, the plurality of particles have an average diameter between about 10 nm and about 500 nm, between about 50 nm and about 400 nm, between about 100 nm and about 300 nm, between about 150 nm and about 250 nm, between about 175 nm and about 225 nm, or the like. In some embodiments, the plurality of particles have an average diameter between about 10 nm and about 500 nm, between about 20 nm and about 400 nm, between about 30 nm and about 300 nm, between about 40 nm and about 200 nm, between about 50 nm and about 175 nm, between about 60 nm and about 150 nm, between about 70 nm and about 130 nm, or the like. For example, the average diameter can be between about 70 nm and 130 nm. In some embodiments, the plurality of particles have an average diameter between about 20 nm and about 220 nm, between about 30 nm and about 200 nm, between about 40 nm and about 180 nm, between about 50 nm and about 170 nm, between about 60 nm and about 150 nm, or between about 70 nm and about 130 nm. In one embodiment, the particles have a size of 40 to 120 nm with a zeta potential close to 0 mV at low to zero ionic strengths (1 to 10 mM), with zeta potential values between +5 to −5 mV, and a zero/neutral or a small -ve surface charge.

In some embodiments, the particles of the invention may comprise more than one conjugates. The conjugates may be different, e.g., comprising different payloads. In some embodiments, the particles of the invention may comprises conjugates having different PK values. Conjugates in the same particle are protected by the particle and are released at the same time. In some embodiments, linkers of the conjugates are cleaved under the same condition and payloads of the conjugates are released at the same time. In some embodiments, linkers of the conjugates are cleaved under different conditions and payloads of the conjugates are released sequentially. In one particlular embodiment, the particles of the invention may comprise a first conjugate having immune stimulating agents as payloads and a second conjugate having antigens as payloads. The linkers of the first conjugate are cleaved before the linkers of the second conjugate, thereby releaving the immune stimulating agents and then the antigens.

In some embodiments, the weight percentage of the conjugate in the particles is at least about 0.05%, 0.1%, 0.5%, 1%, 5%, 10%, 15%, 20%, 25%, 30%, 35%, 40%, 45%, or 50% such that the sum of the weight percentages of the components of the particles is 100%. In some embodiments, the weight percentage of the conjugate in the particles is from about 0.5% to about 10%, or about 10% to about 20%, or about 20% to about 30%, or about 30% to about 40%, or about 40% to about 50%, or about 50% to about 60%, or about 60% to about 70%, or about 70% to about 80%, or about 80% to about 90%, or about 90% to about 99% such that the sum of the weight percentages of the components of the particles is 100%.

### A. Polymers

The particles of the invention may contain one or more polymers. Polymers may contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide, collectively referred to herein as “PLA”, and caprolactone units, such as poly(ϵ-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker.

The particles may contain one or more hydrophilic polymers. Hydrophilic polymers include cellulosic polymers such as starch and polysaccharides; hydrophilic polypeptides; poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol); polyoxazoline; and copolymers thereof.

The particles may contain one or more hydrophobic polymers. Examples of suitable hydrophobic polymers include polyhydroxyacids such as poly(lactic acid), poly(glycolic acid), and poly(lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof.

In certain embodiments, the hydrophobic polymer is an aliphatic polyester. In some embodiments, the hydrophobic polymer is poly(lactic acid), poly(glycolic acid), or poly(lactic acid-co-glycolic acid).

The particles can contain one or more biodegradable polymers. Biodegradable polymers can include polymers that are insoluble or sparingly soluble in water that are converted chemically or enzymatically in the body into water-soluble materials. Biodegradable polymers can include soluble polymers crosslinked by hydolyzable cross-linking groups to render the crosslinked polymer insoluble or sparingly soluble in water.

Biodegradable polymers in the particle can include polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose such as methyl cellulose and ethyl cellulose, hydroxyalkyl celluloses such as hydroxypropyl cellulose, hydroxy-propyl methyl cellulose, and hydroxybutyl methyl cellulose, cellulose ethers, cellulose esters, nitro celluloses, cellulose acetate, cellulose propionate, cellulose acetate butyrate, cellulose acetate phthalate, carboxylethyl cellulose, cellulose triacetate, cellulose sulphate sodium salt, polymers of acrylic and methacrylic esters such as poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene and polyvinylpryrrolidone, derivatives thereof, linear and branched copolymers and block copolymers thereof, and blends thereof. Exemplary biodegradable polymers include polyesters, poly(ortho esters), poly(ethylene imines), poly(caprolactones), poly(hydroxyalkanoates), poly(hydroxyvalerates), polyanhydrides, poly(acrylic acids), polyglycolides, poly(urethanes), polycarbonates, polyphosphate esters, polyphosphazenes, derivatives thereof, linear and branched copolymers and block copolymers thereof, and blends thereof. In some embodiments the particle contains biodegradable polyesters or polyanhydrides such as poly(lactic acid), poly(glycolic acid), and poly(lactic-co-glycolic acid).

The particles can contain one or more amphiphilic polymers. Amphiphilic polymers can be polymers containing a hydrophobic polymer block and a hydrophilic polymer block. The hydrophobic polymer block can contain one or more of the hydrophobic polymers above or a derivative or copolymer thereof. The hydrophilic polymer block can contain one or more of the hydrophilic polymers above or a derivative or copolymer thereof. In some embodiments the amphiphilic polymer is a di-block polymer containing a hydrophobic end formed from a hydrophobic polymer and a hydrophilic end formed of a hydrophilic polymer. In some embodiments, a moiety can be attached to the hydrophobic end, to the hydrophilic end, or both. The particle can contain two or more amphiphilic polymers.

### B. Lipids

The particles may contain one or more lipids or amphiphilic compounds. For example, the particles can be liposomes, lipid micelles, solid lipid particles, or lipid-stabilized polymeric particles. The lipid particle can be made from one or a mixture of different lipids. Lipid particles are formed from one or more lipids, which can be neutral, anionic, or cationic at physiologic pH. The lipid particle is preferably made from one or more biocompatible lipids. The lipid particles may be formed from a combination of more than one lipid, for example, a charged lipid may be combined with a lipid that is non-ionic or uncharged at physiological pH.

The particle can be a lipid micelle. Lipid micelles for drug delivery are known in the art. Lipid micelles can be formed, for instance, as a water-in-oil emulsion with a lipid surfactant. An emulsion is a blend of two immiscible phases wherein a surfactant is added to stabilize the dispersed droplets. In some embodiments the lipid micelle is a microemulsion. A microemulsion is a thermodynamically stable system composed of at least water, oil and a lipid surfactant producing a transparent and thermodynamically stable system whose droplet size is less than 1 micron, from about 10 nm to about 500 nm, or from about 10 nm to about 250 nm. Lipid micelles are generally useful for encapsulating hydrophobic active agents, including hydrophobic therapeutic agents, hydrophobic prophylactic agents, or hydrophobic diagnostic agents.

The particle can be a liposome. Liposomes are small vesicles composed of an aqueous medium surrounded by lipids arranged in spherical bilayers. Liposomes can be classified as small unilamellar vesicles, large unilamellar vesicles, or multi-lamellar vesicles. Multi-lamellar liposomes contain multiple concentric lipid bilayers. Liposomes can be used to encapsulate agents, by trapping hydrophilic agents in the aqueous interior or between bilayers, or by trapping hydrophobic agents within the bilayer.

The lipid micelles and liposomes typically have an aqueous center. The aqueous center can contain water or a mixture of water and alcohol. Suitable alcohols include, but are not limited to, methanol, ethanol, propanol, (such as isopropanol), butanol (such as n-butanol, isobutanol, sec-butanol, tert-butanol, pentanol (such as amyl alcohol, isobutyl carbinol), hexanol (such as 1-hexanol, 2-hexanol, 3-hexanol), heptanol (such as 1-heptanol, 2-heptanol, 3-heptanol and 4-heptanol) or octanol (such as 1-octanol) or a combination thereof.

The particle can be a solid lipid particle. Solid lipid particles present an alternative to the colloidal micelles and liposomes. Solid lipid particles are typically submicron in size, i.e. from about 10 nm to about 1 micron, from 10 nm to about 500 nm, or from 10 nm to about 250 nm. Solid lipid particles are formed of lipids that are solids at room temperature. They are derived from oil-in-water emulsions, by replacing the liquid oil by a solid lipid.

Suitable neutral and anionic lipids include, but are not limited to, sterols and lipids such as cholesterol, phospholipids, lysolipids, lysophospholipids, sphingolipids or pegylated lipids. Neutral and anionic lipids include, but are not limited to, phosphatidylcholine (PC) (such as egg PC, soy PC), including 1,2-diacyl-glycero-3-phosphocholines; phosphatidylserine (PS), phosphatidylglycerol, phosphatidylinositol (PI); glycolipids; sphingophospholipids such as sphingomyelin and sphingoglycolipids (also known as 1-ceramidyl glucosides) such as ceramide galactopyranoside, gangliosides and cerebrosides; fatty acids, sterols, containing a carboxylic acid group for example, cholesterol; 1,2-diacyl-sn-glycero-3-phosphoethanolamine, including, but not limited to, 1,2-dioleylphosphoethanolamine (DOPE), 1,2-dihexadecylphosphoethanolamine (DHPE), 1,2-distearoylphosphatidylcholine (DSPC), 1,2-dipalmitoyl phosphatidylcholine (DPPC), and 1,2-dimyristoylphosphatidylcholine (DMPC). The lipids can also include various natural (e.g., tissue derived L-α-phosphatidyl: egg yolk, heart, brain, liver, soybean) and/or synthetic (e.g., saturated and unsaturated 1,2-diacyl-sn-glycero-3-phosphocholines, 1-acyl-2-acyl-sn-glycero-3-phosphocholines, 1,2-diheptanoyl-SN-glycero-3-phosphocholine) derivatives of the lipids.

Suitable cationic lipids include, but are not limited to, N-[1-(2,3-dioleoyloxy)propyl]-N,N,N-trimethyl ammonium salts, also references as TAP lipids, for example methylsulfate salt. Suitable TAP lipids include, but are not limited to, DOTAP (dioleoyl-), DMTAP (dimyristoyl-), DPTAP (dipalmitoyl-), and DSTAP (distearoyl-). Suitable cationic lipids in the liposomes include, but are not limited to, dimethyldioctadecyl ammonium bromide (DDAB), 1,2-diacyloxy-3-trimethylammonium propanes, N-[1-(2,3-dioloyloxy)propyl]-N,N-dimethyl amine (DODAP), 1,2-diacyloxy-3-dimethylammonium propanes, N-[1-(2,3-dioleyloxy)propyl]-N,N,N-trimethylammonium chloride (DOTMA), 1,2-dialkyloxy-3-dimethylammonium propanes, dioctadecylamidoglycylspermine (DOGS), 3-[N-(N′,N′-dimethylamino-ethane)carbamoyl]cholesterol (DC-Chol); 2,3-dioleoyloxy-N-(2-(sperminecarboxamido)-ethyl)-N,N-dimethyl-1-propanaminium trifluoro-acetate (DOSPA), β-alanyl cholesterol, cetyl trimethyl ammonium bromide (CTAB), diC₁₄-amidine, N-ferf-butyl-N′-tetradecyl-3-tetradecylamino-propionamidine, N-(alpha-trimethylammonioacetyl)didodecyl-D-glutamate chloride (TMAG), ditetradecanoyl-N-(trimethylammonio-acetyl)diethanolamine chloride, 1,3-dioleoyloxy-2-(6-carboxy-spermyl)-propylamide (DOSPER), and N,N,N′,N′-tetramethyl-, N′-bis(2-hydroxylethyl)-2,3-dioleoyloxy-1,4-butanediammonium iodide. In one embodiment, the cationic lipids can be 1-[2-(acyloxy)ethyl]2-alkyl(alkenyl)-3-(2-hydroxyethyl)-imidazolinium chloride derivatives, for example, 1-[2-(9(Z)-octadecenoyloxy)ethyl]-2-(8(Z)-heptadecenyl-3-(2-hydroxyethyl)imidazolinium chloride (DOTIM), and 1-[2-(hexadecanoyloxy)ethyl]-2-pentadecyl-3-(2-hydroxyethyl)imidazolinium chloride (DPTIM). In one embodiment, the cationic lipids can be 2,3-dialkyloxypropyl quaternary ammonium compound derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimetyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

Suitable solid lipids include, but are not limited to, higher saturated alcohols, higher fatty acids, sphingolipids, synthetic esters, and mono-, di-, and triglycerides of higher saturated fatty acids. Solid lipids can include aliphatic alcohols having 10-40, preferably 12-30 carbon atoms, such as cetostearyl alcohol. Solid lipids can include higher fatty acids of 10-40, preferably 12-30 carbon atoms, such as stearic acid, palmitic acid, decanoic acid, and behenic acid. Solid lipids can include glycerides, including monoglycerides, diglycerides, and triglycerides, of higher saturated fatty acids having 10-40, preferably 12-30 carbon atoms, such as glyceryl monostearate, glycerol behenate, glycerol palmitostearate, glycerol trilaurate, tricaprin, trilaurin, trimyristin, tripalmitin, tristearin, and hydrogenated castor oil. Suitable solid lipids can include cetyl palmitate, beeswax, or cyclodextrin.

Amphiphilic compounds include, but are not limited to, phospholipids, such as 1,2 distearoyl-sn-glycero-3-phosphoethanolamine (DSPE), dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), and dilignoceroylphatidylcholine (DLPC), incorporated at a ratio of between 0.01-60 (weight lipid/w polymer), for example, between 0.1-30 (weight lipid/w polymer). Phospholipids which may be used include, but are not limited to, phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and β-acyl-y-alkyl phospholipids. Examples of phospholipids include, but are not limited to, phosphatidylcholines such as dioleoylphosphatidylcholine, dimyristoylphosphatidylcholine, dipentadecanoylphosphatidylcholine dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcho-line (DBPC), ditricosanoylphosphatidylcholine (DTPC), dilignoceroylphatidylcholine (DLPC); and phosphatidylethanolamines such as dioleoylphosphatidylethanolamine or 1-hexadecyl-2-palmitoylglycerophos-phoethanolamine. Synthetic phospholipids with asymmetric acyl chains (e.g., with one acyl chain of 6 carbons and another acyl chain of 12 carbons) may also be used.

### C. Immunological Conjugates

The particles contain one or more immunological conjugates as described above. The conjugates can be present on the interior of the particle, on the exterior of the particle, or both The term “immunological conjugates” as used herein refers of any conjugates that can modulate an immune response in a subject, in particular, an anti-cancer immune response. The conjugates may comprise any combination of the payloads, linkers and targeting moieties as described in the previous sections.

### D. Hydrophobic Ion Pairing Complexes

The particles may comprise hydrophobic ion-pairing complexes or hydrophobic ioin-pairs formed by one or more conjugates described above and counterions.

Hydrophobic ion-pairing (HIP) is the interaction between a pair of oppositely charged ions held together by Coulombic attraction. HIP, as used here in, refers to the interaction between the conjugate of the present invention and its counterions, wherein the counterion is not H⁺ or HO⁻ ions. Hydrophobic ion-pairing complex or hydrophobic ion-pair, as used herein, refers to the complex formed by the conjugate of the present invention and its counterions. In some embodiments, the counterions are hydrophobic. In some embodiments, the counterions are provided by a hydrophobic acid or a salt of a hydrophobic acid. In some embodiments, the counterions are provided by bile acids or salts, fatty acids or salts, lipids, or amino acids. In some embodiments, the counterions are negatively charged (anionic). Non-limited examples of negative charged counterions include the counterions sodium sulfosuccinate (AOT), sodium oleate, sodium dodecyl sulfate (SDS), human serum albumin (HSA), dextran sulphate, sodium deoxycholate, sodium cholate, anionic lipids, amino acids, or any combination thereof. Non-limited examples of positively charged counterions include 1,2-dioleoyl-3-trimethylammonium-propane (chloride salt) (DOTAP), cetrimonium bromide (CTAB), quaternary ammonium salt didodecyl dimethylammonium bromide (DMAB) or Didodecyldimethylammonium bromide (DDAB). Without wishing to be bound by any theory, in some embodiments, HIP may increase the hydrophobicity and/or lipophilicity of the conjugate of the present invention. In some embodiments, increasing the hydrophobicity and/or lipophilicity of the conjugate of the present invention may be beneficial for particle formulations and may provide higher solubility of the conjugate of the present invention in organic solvents. Without wishing to be bound by any theory, it is believed that particle formulations that include HIP pairs have improved formulation properties, such as drug loading and/or release profile. Without wishing to be bound by any theory, in some embodiments, slow release of the conjugate of the invention from the particles may occur, due to a decrease in the conjugate's solubility in aqueous solution. In addition, without wishing to be bound by any theory, complexing the conjugate with large hydrophobic counterions may slow diffusion of the conjugate within a polymeric matrix. In some emobodiments, HIP occurs without covalent conjuatation of the counterion to the conjugate of the present invention.

Without wishing to be bound by any theory, the strength of HIP may impact the drug load and release rate of the particles of the invention. In some embodiments, the strength of the HIP may be increased by increasing the magnitude of the difference between the pKa of the conjugate of the present invention and the pKa of the agent providing the counterion. Also without wishing to be bound by any theory, the conditions for ion pair formation may impact the drug load and release rate of the particles of the invention.

In some embodiments, any suitable hydrophobic acid or a combination thereof may form a HIP pair with the conjugate of the present invention. In some embodiments, the hydrophobic acid may be a carboxylic acid (such as but not limited to a monocarboxylic acid, dicarboxylic acid, tricarboxylic acid), a sulfinic acid, a sulfenic acid, or a sulfonic acid. In some embodiments, a salt of a suitable hydrophobic acid or a combination thereof may be used to form a HIP pair with the conjugate of the present invention. Examples of hydrophobic acids, saturated fatty acids, unsaturated fatty acids, aromatic acids, bile acid, polyelectrolyte, their dissociation constant in water (pKa) and logP values were disclosed in WO2014/043,625, the content of which is incorporated herein by reference in its entirety. The strength of the hydrophobic acid, the difference between the pKa of the hydrophobic acid and the pKa of the conjuagate of the present invention, logP of the hydrophobic acid, the phase transition temperature of the hydrophobic acid, the molar ratio of the hydrophobic acid to the conjugate of the present invention, and the concentration of the hydrophobic acid were also disclosed in WO2014/043,625, the content of which is incorporated herein by reference in its entirety.

In some embodiments, particles of the present invention comprising a HIP complex and/or prepared by a process that provides a counterion to form HIP complex with the conjugate may have a higher drug loading than particles without a HIP complex or prepared by a process that does not provide any counterion to form HIP complex with the conjugate. In some embodiments, drug loading may increase 50%, 100%, 2 times, 3 times, 4 times, 5 times, 6 times, 7 times, 8 times, 9 times, or 10 times.

In some embodiments, the particles of the invention may retain the conjugate for at least about 1 minute, at least about 15 minutes, at least about 1 hour, when placed in a phosphate buffer solution at 37° C.

### E. Immunological Adjuvants

The particles may further comprise one or more immunologic adjuvants. As used herein, the term “immunologic adjuvant” refers to a compound or a mixture of compounds that acts to accelerate, prolong, enhance or modify immune responses when used in conjugation with an immunogen (e.g., neoantigens). Adjuvant may be non-immunogenic when administered to a host alone, but that augments the host's immune response to another antigen when administered conjointly with that antigen. Specifically, the terms “adjuvant” and “immunologic adjuvant” are used interchangeably in the present invention. Adjuvant-mediated enhancement and/or extension of the duration of the immune response can be assessed by any method known in the art including without limitation one or more of the following: (i) an increase in the number of antibodies produced in response to immunization with the adjuvant/antigen combination versus those produced in response to immunization with the antigen alone; (ii) an increase in the number of T cells recognizing the antigen or the adjuvant; and (iii) an increase in the level of one or more cytokines.

Adjuvants may be aluminium based adjuvants including but not limiting to aluminium hydroxide and aluminium phosphate; saponins such as steroid saponins and triterpenold saponins; bacterial flagellin and some cytokines such as GM-CSF. Adjuvants selection may depend on antigens, vaccines and routes of administrations.

Adjuvants may include, but are not limited to, alpha glucose bearing glycosphingolipid compounds disclosed by Chen et al (US Patent publication NO. 2015/0071960, the content of which is incorporated herein by reference in its entirety). Those compounds when added into the present particles in combination with conjugates of the present invention, can elevate invariant natural killer T (iNKT) cells and increases cytokine and/or chemokine production, where the cytokine production is sufficient to transactivate downstream immune cells including dendritic cells, natural killer cells, B cells, CD+4 T and CD8+ T cells.

In some embodiments, adjuvants improve the adaptive immune response to a vaccine antigen by modulating innate immunity or facilitating transport and presentation. Adjuvants act directly or indirectly on antigen presenting cells (APCs) including dendritic cells (DCs). Adjuvants may be ligands for toll-like receptors (TLRs) and can directly affect DCs to alter the strength, potency, speed, duration, bias, breadth, and scope of adaptive immunity. In other instances, adjuvants may signal via proinflammatory pathways and promote immune cell infiltration, antigen presentation, and effector cell maturation. This class of adjuvants includes mineral salts, oil emulsions, nanoparticles, and polyelectrolytes and comprises colloids and molecular assemblies exhibiting complex, heterogeneous structures (Powell et al., Clin Exp. Vaccine Res., Polyionic vaccine adjuvants: another look at aluminum salts and polyelectrolytes. 2015, 4(1):23-45).

In one example, the particles further comprise pidotimod as an adjuvant.

### F. Additional Active Agents

The particles can contain one or more additional active agents in addition to those in the conjugates. The additional active agents can be therapeutic, prophylactic, diagnostic, or nutritional agents as listed above. The additional active agents can be present in any amount, e.g. from about 1% to about 90%, from about 1% to about 50%, from about 1% to about 25%, from about 1% to about 20%, from about 1% to about 10%, or from about 5% to about 10% (w/w) based upon the weight of the particle. In one embodiment, the agents are incorporated in a about 1% to about 10% loading w/w.

### G. Additional Targeting Moieties

The particles can contain one or more targeting moieties targeting the particle to a specific organ, tissue, cell type, or subcellular compartment in addition to the targeting moieties of the conjugate. The additional targeting moieties can be present on the surface of the particle, on the interior of the particle, or both. The additional targeting moieties can be immobilized on the surface of the particle, e.g., can be covalently attached to polymer or lipid in the particle. In preferred embodiments, the additional targeting moieties are covalently attached to an amphiphilic polymer or a lipid such that the targeting moieties are oriented on the surface of the particle.

### III. Pharmaceutical Formulations and Vaccines

In some embodiments, conjugates, particles of the present invention may be formulated as vaccines, provided as liquid suspensions or as freeze-dried products. Suitable liquid preparations may include, but are not limited to, isotonic aqueous solutions, suspensions, emulsions, or viscous compositions that are buffered to a selected pH.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit. As used herein, the term “active ingredient” refers to any chemical and biological substance that has a physiological effect in human or in animals, when exposed to it. In the context of the present invention, the active ingredient in the formulations may be any conjugates and particles as discussed herein above.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

The conjugates or particles of the present invention can be formulated using one or more excipients to: (1) increase stability; (2) permit the sustained or delayed release (e.g., from a depot formulation of the monomaleimide); (3) alter the biodistribution (e.g., target the monomaleimide compounds to specific tissues or cell types); (4) alter the release profile of the monomaleimide compounds in vivo. Non-limiting examples of the excipients include any and all solvents, dispersion media, diluents, or other liquid vehicles, dispersion or suspension aids, surface active agents, isotonic agents, thickening or emulsifying agents, and preservatives. Excipients of the present invention may also include, without limitation, lipidoids, liposomes, lipid nanoparticles, polymers, lipoplexes, core-shell nanoparticles, peptides, proteins, hyaluronidase, nanoparticle mimics and combinations thereof. Accordingly, the formulations of the invention may include one or more excipients, each in an amount that together increases the stability of the monomaleimide compounds.

In some embodiments, the conjugates or particles of the present invention are formulated in aqueous formulations such as pH 7.4 phosphate-buffered formulation, or pH 6.2 citrate-buffered formulation; formulations for lyophilization such as pH 6.2 citrate-buffered formulation with 3% mannitol, pH 6.2 citrate-buffered formulation with 4% mannitol/1% sucrose; or a formulation prepared by the process disclosed in U.S. Pat. No. 8,883,737 to Reddy et al. (Endocyte), the contents of which are incorporated herein by reference in their entirety.

In some embodiments, the conjugates or particles of the present invention targets folate receptors and are formulated in liposomes prepared following methods by Leamon et al. in Bioconjugate Chemistry, vol.14 738-747 (2003), the contents of which are incorporated herein by reference in their entirety. Briefly, folate-targeted liposomes will consist of 40 mole % cholesterol, either 4 mole % or 6 mole % polyethylene glycol (Mr~2000)-derivatized phosphatidylethanolarnine (PEG2000-PE, Nektar, Ala., Huntsville, Ala.), either 0.03 mole % or 0.1 mole % folate-cysteine-PE(13400-PE and the remaining mole % will be composed of egg phosphatidylcholine, as disclosed in U.S. Pat. No. 8,765,096 to Leamon et al. (Endocyte), the contents of which are incorporated herein by reference in their entirety. Lipids in chloroform will be dried to a thin film by rotary evaporation and then rehydrated in PBS containing the drug. Rehydration will be accomplished by vigorous vortexing followed by 10 cycles of freezing and thawing. Liposomes will be extruded 10 times through a 50 nm pore size polycarbonate membrane using a high-pressure extruder. Similarly, liposomes not targeting folate receptors may be prepared identically with the absence of folate-cysteine-PEG3400-PE.

In some embodiments, the conjugates or particles of the present invention are formulated in parenteral dosage forms including but limited to aqueous solutions of the conjugates or particles, in an isotonic saline, 5% glucose or other pharmaceutically acceptable liquid carriers such as liquid alcohols, glycols, esters, and amides, as disclosed in U.S. Pat. No. 7,910,594 to Vlahov et al. (Endocyte), the contents of which are incorporated herein by reference in their entirety. The parenteral dosage form may be in the form of a reconstitutable lyophilizate comprising the dose of the conjugates or particles. Any prolonged release dosage forms known in the art can be utilized such as, for example, the biodegradable carbohydrate matrices described in U.S. Pat. Nos. 4,713,249; 5,266,333; and 5,417,982, the disclosures of which are incorporated herein by reference, or, alternatively, a slow pump (e.g., an osmotic pump) can be used.

In some embodiments, the parenteral formulations are aqueous solutions containing carriers or excipients such as salts, carbohydrates and buffering agents (e.g., at a pH of from 3 to 9). In some embodiments, the conjugates or particles of the present invention may be formulated as a sterile non-aqueous solution or as a dried form and may be used in conjunction with a suitable vehicle such as sterile, pyrogen-free water. The preparation of parenteral formulations under sterile conditions, for example, by lyophilization under sterile conditions, may readily be accomplished using standard pharmaceutical techniques well-known to those skilled in the art. The solubility of a conjugates or particles used in the preparation of a parenteral formulation may be increased by the use of appropriate formulation techniques, such as the incorporation of solubility-enhancing agents.

In some embodiments, the conjugates or particles of the present invention may be prepared in an aqueous sterile liquid formulation comprising monobasic sodium phosphate monohydrate, dibasic disodium phosphate dihydrate, sodium chloride, potassium chloride and water for injection, as disclosed in US 20140140925 to Leamon et al., the contents of which are incorporated herein by reference in their entirety. For example, the conjugates or particles of the present invention may be formulated in an aqueous liquid of pH 7.4, phosphate buffered formulation for intravenous administration as disclosed in Example 23 of WO2011014821 to Leamon et al. (Endocyte), the contents of which are incorporated herein by reference in their entirety. According to Leamon, the aqueous formulation needs to be stored in the frozen state to ensure its stability.

In some embodiments, the conjugates or particles of the present invention are formulated for intravenous (IV) administration. Any formulation or any formulation prepared according to the process disclosed in US 20140030321 to Ritter et al. (Endocyte), the contents of which are incorporated herein by reference in their entirety, may be used. For example, the conjugates or particles may be formulated in an aqueous sterile liquid formulation of pH 7.4 phosphate buffered composition comprising sodium phosphate, monobasic monohydrate, disodium phosphate, dibasic dehydrate, sodium chloride, and water for injection. As another example, the conjugates or particles may be formulated in pH 6.2 citrated-buffered formulation comprising trisodium citrate, dehydrate, citric acid and water for injection. As another example, the conjugates or particles may be formulated with 3% mannitol in a pH 6.2 citrate-buffered formulation for lyophilization comprising trisodium citrate, dehydrate, citric acid and mannitol. 3% mannitol may be replaced with 4% mannitol and 1% sucrose.

In some embodiments, the particles comprise biocompatible polymers. In some embodiments, the particles comprise about 0.2 to about 35 weight percent of a therapeutic agent; and about 10 to about 99 weight percent of a biocompatible polymer such as a diblock poly(lactic) acid-poly(ethylene)glycol as disclosed in US 20140356444 to Troiano et al. (BIND Therapeutics), the contents of which are incorporated herein by reference in their entirety. Any therapeutically particle composition in U.S. Pat. Nos. 8,663,700, 8,652,528, 8,609,142, 8,293,276 and 8,420,123, the contents of each of which are incorporated herein by reference in their entirety, may also be used.

In some embodiments, the particles comprise a hydrophobic acid. In some embodiments, the particles comprise about 0.05 to about 30 weight percent of a substantially hydrophobic acid; about 0.2 to about 20 weight percent of a basic therapeutic agent having a protonatable nitrogen; wherein the pKa of the basic therapeutic agent is at least about 1.0 pKa units greater than the pKa of the hydrophobic acid; and about 50 to about 99.75 weight percent of a diblock poly(lactic) acid-poly(ethylene)glycol copolymer or a diblock poly(lactic acid-co-glycolic acid)-poly(ethylene)glycol copolymer, wherein the therapeutic nanoparticle comprises about 10 to about 30 weight percent poly(ethylene)glycol as disclosed in WO2014043625 to Figueiredo et al. (BIND Therapeutics), the contents of which are incorporated herein by reference in their entirety. Any therapeutical particle composition in US 20140149158, 20140248358, 20140178475 to Figueiredo et al., the contents of each of which are incorporated herein by reference in their entirety, may also be used.

In some embodiments, the particles comprise a chemotherapeutic agent; a diblock copolymer of poly(ethylene)glycol and polylactic acid; and a ligand conjugate, as disclosed in US 20140235706 to Zale et al. (BIND Therapeutics), the contents of which are incorporated herein by reference in their entirety. Any of the particle compositions in U.S. Pat. Nos. 8,603,501, 8,603,500, 8,603,499, 8,273,363, 8,246,968, 20130172406 to Zale et al., may also be used.

In some embodiments, the particles comprise a targeting moiety. As a non-limiting example, the particles may comprise about 1 to about 20 mole percent PLA-PEG-basement vascular membrane targeting peptide, wherein the targeting peptide comprises PLA having a number average molecular weight of about 15 to about 20 kDa and PEG having a number average molecular weight of about 4 to about 6 kDa; about 10 to about 25 weight percent anti-neointimal hyperplasia (NIH) agent; and about 50 to about 90 weight percent non-targeted poly-lactic acid-PEG, wherein the therapeutic particle is capable of releasing the anti-NIH agent to a basement vascular membrane of a blood vessel for at least about 8 hours when the therapeutic particle is placed in the blood vessel as disclosed in U.S. Pat. No. 8,563,041 to Grayson et al. (BIND Therapeutics), the contents of which are incorporated herein by reference in their entirety.

In some embodiments, the particles comprise about 4 to about 25% by weight of an anti-cancer agent; about 40 to about 99% by weight of poly(D,L-lactic)acid-poly(ethylene)glycol copolymer; and about 0.2 to about 10 mole percent PLA-PEG-ligand; wherein the pharmaceutical aqueous suspension have a glass transition temperature between about 39 and 41° C., as disclosed in U.S. Pat. No. 8,518,963 to Ali et al. (BIND Therapeutics), the contents of which are incorporated herein by reference in their entirety.

In some embodiments, the particles comprise about 0.2 to about 35 weight percent of a therapeutic agent; about 10 to about 99 weight percent of a diblock poly(lactic) acid-poly(ethylene)glycol copolymer or a diblock poly(lactic)-co-poly (glycolic) acid-poly(ethylene)glycol copolymer; and about 0 to about 75 weight percent poly(lactic) acid or poly(lactic) acid-co-poly (glycolic) acid as disclosed in WO2012166923 to Zale et al. (BIND Therapeutics), the contents of which are incorporated herein by reference in their entirety.

In some embodiments, the particles are long circulating and may be formulated in a biocompatible and injectable formulation. For example, the particles may be a sterile, biocompatible and injectable nanoparticle composition comprising a plurality of long circulating nanoparticles having a diameter of about 70 to about 130 nm, each of the plurality of the long circulating nanoparticles comprising about 70 to about 90 weight percent poly(lactic) acid-co-poly(ethylene) glycol, wherein the weight ratio of poly(lactic) acid to poly(ethylene) glycol is about 15 kDa/2 kDa to about 20 kDa/10 kDa, and a therapeutic agent encapsulated in the nanoparticles as disclosed in US 20140093579 to Zale et al. (BIND Therapeutics), the content of which is incorporated herein by reference in its entirety.

In some embodiments, provided is a reconstituted lyophilized pharmaceutical composition suitable for parenteral administration comprising the particles of the present invention. For example, the reconstituted lyophilized pharmaceutical composition may comprise a 10-100 mg/mL concentration of polymeric nanoparticles in an aqueous medium; wherein the polymeric nanoparticles comprise: a poly(lactic) acid-block-poly(ethylene)glycol copolymer or poly(lactic)-co-poly(glycolic) acid-block-poly(ethylene)glycol copolymer, and a taxane agent; 4 to 6 weight percent sucrose or trehalose; and 7 to 12 weight percent hydroxypropyl β-cyclodextrin, as disclosed in U.S. Pat. No. 8,637,083 to Troiano et al. (BIND Therapeutics), the contents of which are incorporated herein by reference in their entirety. Any pharmaceutical composition in U.S. Pat. Nos. 8,603,535, 8,357,401, 20130230568, 20130243863 to Troiano et al. may also be used.

In some embodiments, the conjugates and/or particles of the invention may be delivered with a bacteriophage. For example, a bacteriophage may be conjugated through a labile/non labile linker or directly to at least 1,000 therapeutic drug molecules such that the drug molecules are conjugated to the outer surface of the bacteriophage as disclosed in US 20110286971 to Yacoby et al., the content of which is incorporated herein by reference in its entirety. According to Yacoby et al., the bacteriophage may comprise an exogenous targeting moiety that binds a cell surface molecule on a target cell.

In some embodiments, the conjugates and/or particles of the invention may be delivered with a dendrimer. The conjugates may be encapsulated in a dendrimer, or disposed on the surface of a dendrimer. For example, the conjugates may bind to a scaffold for dendritic encapsulation, wherein the scaffold is covalently or non-covalently attached to a polysaccharide, as disclosed in US 20090036553 to Piccariello et al., the content of which is incorporated herein by reference in its entirety. The scaffold may be any peptide or oligonucleotide scaffold disclosed by Piccariello et al.

In some embodiments, the conjugates and/or particles of the invention may be delivered by a cyclodextrin. In one embodiment, the conjugates may be formulated with a polymer comprising a cyclodextrin moiety and a linker moiety as disclosed in US 20130288986 to Davis et al., the content of which is incorporated herein by reference in its entirety. Davis et al. also teaches that the conjugate may be covalently attached to a polymer through a tether, wherein the tether comprises a self-cyclizing moiety.

In some embodiments, the conjugates and/or particles of the invention may be delivered with an aliphatic polymer. For example, the aliphatic polymer may comprise polyesters with grafted zwitterions, such as polyester-graft-phosphorylcholine polymers prepared by ring-opening polymerization and click chemistry as disclosed in U.S. Pat. No. 8,802,738 to Emrick; the content of which is incorporated herein by reference in its entirety.

### A. Excipients

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, Md., 2006; incorporated herein by reference in its entirety) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEENn®60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ®45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC®F 68, POLOXAMER®188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN®II, NEOLONE™, KATHON™, and/or EUXYL®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition, according to the judgment of the formulator.

### B. Lipidoids

Lipidoids may be used to deliver conjugates of the present invention. Complexes, micelles, liposomes or particles can be prepared containing these lipidoids and therefore, can result in an effective delivery of the conjugates of the present invention, for a variety of therapeutic indications including vaccine adjuvants, following the injection of a lipidoid formulation via localized and/or systemic routes of administration. Lipidoid complexes of conjugates of the present invention can be administered by various means including, but not limited to, intravenous, intramuscular, or subcutaneous routes.

The lipidoid formulations can include particles comprising either 3 or 4 or more components in addition to conjugates of the present invention.

The use of lipidoid formulations for the localized delivery of conjugates to cells (such as, but not limited to, adipose cells and muscle cells) via either subcutaneous or intramuscular delivery, may not require all of the formulation components desired for systemic delivery, and as such may comprise only the lipidoid and the conjugates.

### C. Liposomes, Lipid Nanoparticles and Lipoplexes

The conjugates of the invention can be formulated using one or more liposomes, lipoplexes, or lipid nanoparticles. In one embodiment, pharmaceutical compositions of the conjugates of the invention include liposomes. Liposomes are artificially-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In one embodiment, pharmaceutical compositions described herein may include, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety).

In one embodiment, the conjugates of the invention may be formulated in a lipid vesicle which may have crosslinks between functionalized lipid bilayers.

In one embodiment, the conjugates of the invention may be formulated in a lipid-polycation complex. The formation of the lipid-polycation complex may be accomplished by methods known in the art and/or as described in U.S. Pub. No. 20120178702, herein incorporated by reference in its entirety. As a non-limiting example, the polycation may include a cationic peptide or a polypeptide such as, but not limited to, polylysine, polyornithine and/or polyarginine and the cationic peptides described in International Pub. No. WO2012013326; herein incorporated by reference in its entirety. In another embodiment, the conjugates of the invention may be formulated in a lipid-polycation complex which may further include a neutral lipid such as, but not limited to, cholesterol or dioleoyl phosphatidylethanolamine (DOPE).

The liposome formulation may be influenced by, but not limited to, the selection of the cationic lipid component, the degree of cationic lipid saturation, the nature of the PEGylation, ratio of all components and biophysical parameters such as size.

In one embodiment, the cationic lipid may be selected from, but not limited to, a cationic lipid described in International Publication Nos. WO2012040184, WO2011153120, WO2011149733, WO2011090965, WO2011043913, WO2011022460, WO2012061259, WO2012054365, WO2012044638, WO2010080724, WO201021865 and WO2008103276, U.S. Pat. Nos. 7,893,302, 7,404,969 and 8,283,333 and US Patent Publication No. US20100036115 and US20120202871; each of which is herein incorporated by reference in their entirety. In another embodiment, the cationic lipid may be selected from, but not limited to, formula A described in International Publication Nos. WO2012040184, WO2011153120, WO2011149733, WO2011090965, WO2011043913, WO2011022460, WO2012061259, WO2012054365 and WO2012044638; each of which is herein incorporated by reference in their entirety. In yet another embodiment, the cationic lipid may be selected from, but not limited to, formula CLI-CLXXIX of International Publication No. WO2008103276, formula CLI-CLXXIX of U.S. Pat. No. 7,893,302, formula CLI-CLXXXXI of U.S. Pat. No. 7,404,969 and formula I-VI of US Patent Publication No. US20100036115; the contents of each of which are herein incorporated by reference in their entirety.

In one embodiment, the cationic lipid may be synthesized by methods known in the art and/or as described in International Publication Nos. WO2012040184, WO2011153120, WO2011149733, WO2011090965, WO2011043913, WO2011022460, WO2012061259, WO2012054365, WO2012044638, WO2010080724 and WO201021865; each of which is herein incorporated by reference in their entirety.

In one embodiment, the LNP formulation may be formulated by the methods described in International Publication Nos. WO2011127255 or WO2008103276, each of which is herein incorporated by reference in their entirety. As a non-limiting example, conjugates described herein may be encapsulated in LNP formulations as described in WO2011127255 and/or WO2008103276; each of which is herein incorporated by reference in their entirety. As another non-limiting example, conjugates described herein may be formulated in a nanoparticle to be delivered by a parenteral route as described in U.S. Pub. No. 20120207845; herein incorporated by reference in its entirety.

The nanoparticle formulations may be a carbohydrate nanoparticle comprising a carbohydrate carrier and a conjugate. As a non-limiting example, the carbohydrate carrier may include, but is not limited to, an anhydride-modified phytoglycogen or glycogen-type material, phtoglycogen octenyl succinate, phytoglycogen beta-dextrin, anhydride-modified phytoglycogen beta-dextrin. (See e.g., International Publication No. WO2012109121; herein incorporated by reference in its entirety).

Nanoparticles may be engineered to alter the surface properties of particles so the lipid nanoparticles may penetrate the mucosal barrier. Mucus is located on mucosal tissue such as, but not limited to, oral (e.g., the buccal and esophageal membranes and tonsil tissue), ophthalmic, gastrointestinal (e.g., stomach, small intestine, large intestine, colon, rectum), nasal, respiratory (e.g., nasal, pharyngeal, tracheal and bronchial membranes), genital (e.g., vaginal, cervical and urethral membranes). Nanoparticles larger than 10-200 nm which are preferred for higher drug encapsulation efficiency and the ability to provide the sustained delivery of a wide array of drugs have been thought to be too large to rapidly diffuse through mucosal barriers. Mucus is continuously secreted, shed, discarded or digested and recycled so most of the trapped particles may be removed from the mucosa tissue within seconds or within a few hours. Large polymeric nanoparticles (200nm -500 nm in diameter) which have been coated densely with a low molecular weight polyethylene glycol (PEG) diffused through mucus only 4 to 6-fold lower than the same particles diffusing in water (Lai et al. PNAS 2007 104(5):1482-487; Lai et al. Adv Drug Deliv Rev. 2009 61(2): 158-171; each of which is herein incorporated by reference in their entirety). The transport of nanoparticles may be determined using rates of permeation and/or fluorescent microscopy techniques including, but not limited to, fluorescence recovery after photo bleaching (FRAP) and high resolution multiple particle tracking (MPT). As a non-limiting example, compositions which can penetrate a mucosal barrier may be made as described in U.S. Pat. No. 8,241,670, herein incorporated by reference in its entirety.

Nanoparticle engineered to penetrate mucus may comprise a polymeric material (i.e. a polymeric core) and/or a polymer-vitamin conjugate and/or a tri-block co-polymer. The polymeric material may include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates. The polymeric material may be biodegradable and/or biocompatible. The polymeric material may additionally be irradiated. As a non-limiting example, the polymeric material may be gamma irradiated (See e.g., International App. No. WO201282165, herein incorporated by reference in its entirety). Non-limiting examples of specific polymers include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poly(ortho)esters, poly(butyric acid), poly(valeric acid), poly(lactide-co-caprolactone), and trimethylene carbonate, polyvinylpyrrolidone. The nanoparticle may be coated or associated with a co-polymer such as, but not limited to, a block co-polymer, and (poly(ethylene glycol))-(poly(propylene oxide))-(poly(ethylene glycol)) triblock copolymer (see e.g., US Publication 20120121718 and US Publication 20100003337 and U.S. Pat. No. 8,263,665; each of which is herein incorporated by reference in their entirety). The co-polymer may be a polymer that is generally regarded as safe (GRAS) and the formation of the lipid nanoparticle may be in such a way that no new chemical entities are created. For example, the lipid nanoparticle may comprise poloxamers coating PLGA nanoparticles without forming new chemical entities which are still able to rapidly penetrate human mucus (Yang et al. Angew. Chem. Int. Ed. 2011 50:2597-2600; herein incorporated by reference in its entirety).

The vitamin of the polymer-vitamin conjugate may be vitamin E. The vitamin portion of the conjugate may be substituted with other suitable components such as, but not limited to, vitamin A, vitamin E, other vitamins, cholesterol, a hydrophobic moiety, or a hydrophobic component of other surfactants (e.g., sterol chains, fatty acids, hydrocarbon chains and alkylene oxide chains).

In one embodiment, the conjugate of the invention is formulated as a lipoplex, such as, without limitation, the ATUPLEX™ system, the DACC system, the DBTC system and other conjugate-lipoplex technology from Silence Therapeutics (London, United Kingdom), STEMFECT™ from STEMGENT™ (Cambridge, Mass.), and polyethylenimine (PEI) or protamine-based targeted and non-targeted delivery of therapeutic agents (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293 Weide et al. J Immunother. 2009 32:498-507; Weide et al. J Immunother. 2008 31:180-188; Pascolo, Expert Opin. Biol. Ther. 4:1285-1294; Fotin-Mleczek et al., 2011 J. Immunother. 34:1-15; Song et al., Nature Biotechnol. 2005, 23:709-717; Peer et al., Proc Natl Acad Sci USA. 2007 6;104:4095-4100; deFougerolles Hum Gene Ther. 2008 19:125-132; all of which are incorporated herein by reference in its entirety).

In one embodiment such formulations may also be constructed or compositions altered such that they passively or actively are directed to different cell types in vivo, including but not limited to hepatocytes, immune cells (e.g., antigen presenting cells, dendritic cells, T lymphocytes, B lymphocytes, natural killer cells and leukocytes), tumor cells and endothelial cells, (Akinc et al. Mol Ther. 2010 18:1357-1364; Song et al., Nat Biotechnol. 2005 23:709-717; Judge et al., J Clin Invest. 2009 119:661-673; Kaufmann et al., Microvasc Res 2010 80:286-293; Santel et al., Gene Ther 2006 13:1222-1234; Santel et al., Gene Ther 2006 13:1360-1370; Gutbier et al., Pulm Pharmacol. Ther. 2010 23:334-344; Basha et al., Mol. Ther. 2011 19:2186-2200; Fenske and Cullis, Expert Opin Drug Deliv. 2008 5:25-44; Peer et al., Science. 2008 319:627-630; Peer and Lieberman, Gene Ther. 2011 18:1127-1133; all of which are incorporated herein by reference in its entirety). Formulations can also be selectively targeted through expression of different ligands on their surface as exemplified by, but not limited by, folate, transferrin, N-acetylgalactosamine (GalNAc), and antibody targeted approaches (Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011, 12:2708-2714; Zhao et al., Expert Opin Drug Deliv. 2008, 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer, J Control Release. 2010, 20:63-68; Peer et al., Proc Natl Acad Sci USA. 2007, 104:4095-4100; Kim et al., Methods Mol Biol. 2011, 721:339-353; Subramanya et al., Mol Ther. 2010, 18:2028-2037; Song et al., Nat Biotechnol. 2005, 23:709-717; Peer et al., Science. 2008, 319:627-630; Peer and Lieberman, Gene Ther. 2011, 18:1127-1133; all of which are incorporated herein by reference in its entirety).

In one embodiment, the conjugates of the invention are formulated as a solid lipid nanoparticle. A solid lipid nanoparticle (SLN) may be spherical with an average diameter between 10 to 1000 nm. SLN possess a solid lipid core matrix that can solubilize lipophilic molecules and may be stabilized with surfactants and/or emulsifiers. In a further embodiment, the lipid nanoparticle may be a self-assembly lipid-polymer nanoparticle (see Zhang et al., ACS Nano, 2008, 2 (8), pp 1696-1702; herein incorporated by reference in its entirety).

In one embodiment, the conjugates of the invention can be formulated for controlled release and/or targeted delivery. As used herein, “controlled release” refers to a pharmaceutical composition or compound release profile that conforms to a particular pattern of release to effect a therapeutic outcome. In one embodiment, the conjugates of the invention may be encapsulated into a delivery agent described herein and/or known in the art for controlled release and/or targeted delivery. As used herein, the term “encapsulate” means to enclose, surround or encase. As it relates to the formulation of the conjugates of the invention, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of conjugate of the invention may be enclosed, surrounded or encased within the particle. “Partially encapsulation” means that less than 10, 10, 20, 30, 40 50 or less of the conjugate of the invention may be enclosed, surrounded or encased within the particle. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the invention are encapsulated in the particle.

In another embodiment, the conjugates of the invention may be encapsulated into a nanoparticle or a rapidly eliminated nanoparticle and the nanoparticles or a rapidly eliminated nanoparticle may then be encapsulated into a polymer, hydrogel and/or surgical sealant described herein and/or known in the art. As a non-limiting example, the polymer, hydrogel or surgical sealant may be PLGA, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethicon Inc. Cornelia, Ga.), TISSELL® (Baxter International, Inc Deerfield, Ill.), PEG-based sealants, and COSEAL® (Baxter International, Inc Deerfield, Ill.).

In another embodiment, the nanoparticle may be encapsulated into any polymer known in the art which may form a gel when injected into a subject. As a non-limiting example, the nanoparticle may be encapsulated into a polymer matrix which may be biodegradable.

In one embodiment, the conjugate formulation for controlled release and/or targeted delivery may also include at least one controlled release coating. Controlled release coatings include, but are not limited to, OPADRY®, polyvinylpyrrolidone/vinyl acetate copolymer, polyvinylpyrrolidone, hydroxypropyl methylcellulose, hydroxypropyl cellulose, hydroxyethyl cellulose, EUDRAGIT RL®, EUDRAGIT RS® and cellulose derivatives such as ethylcellulose aqueous dispersions (AQUACOAT® and SURELEASE®).

In one embodiment, the controlled release and/or targeted delivery formulation may comprise at least one degradable polyester which may contain polycationic side chains. Degradable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

In one embodiment, the conjugate of the present invention may be encapsulated in a therapeutic nanoparticle. Therapeutic nanoparticles may be formulated by methods described herein and known in the art such as, but not limited to, International Pub Nos. WO2010005740, WO2010030763, WO2010005721, WO2010005723, WO2012054923, US Pub. Nos. US20110262491, US20100104645, US20100087337, US20100068285, US20110274759, US20100068286 and US20120288541, and U.S. Pat. Nos. 8,206,747, 8,293,276 8,318,208 and 8,318,211; each of which is herein incorporated by reference in their entirety. In another embodiment, therapeutic polymer nanoparticles may be identified by the methods described in US Pub No. US20120140790, herein incorporated by reference in its entirety.

In one embodiment, the therapeutic nanoparticle may be formulated for sustained release. As used herein, “sustained release” refers to a pharmaceutical composition or compound that conforms to a release rate over a specific period of time. The period of time may include, but is not limited to, hours, days, weeks, months and years. As a non-limiting example, the sustained release nanoparticle may comprise a polymer and a therapeutic agent such as, but not limited to, the conjugate of the present invention (see International Pub No. 2010075072 and US Pub No. US20100216804, US20110217377 and US20120201859, each of which is herein incorporated by reference in their entirety).

In one embodiment, the therapeutic nanoparticles may be formulated to be target specific. As a non-limiting example, the therapeutic nanoparticles may include a corticosteroid (see International Pub. No. WO2011084518 herein incorporated by reference in its entirety). In one embodiment, the therapeutic nanoparticles of the present invention may be formulated to be antiviral immunotherapeutics or vaccine adjuvants. As a non-limiting example, the therapeutic nanoparticles may be formulated in nanoparticles described in International Pub No. WO2008121949, WO2010005726, WO2010005725, WO2011084521 and US Pub No. US20100069426, US20120004293 and US20100104655, each of which is herein incorporated by reference in their entirety.

In one embodiment, the nanoparticles of the present invention may comprise a polymeric matrix. As a non-limiting example, the nanoparticle may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof.

In one embodiment, the therapeutic nanoparticle comprises a diblock copolymer. In one embodiment, the diblock copolymer may include PEG in combination with a polymer such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof.

As a non-limiting example the therapeutic nanoparticle comprises a PLGA-PEG block copolymer (see US Pub. No. US20120004293 and U.S. Pat. No. 8,236,330, each of which is herein incorporated by reference in their entirety). In another non-limiting example, the therapeutic nanoparticle is a stealth nanoparticle comprising a diblock copolymer of PEG and PLA or PEG and PLGA (see U.S. Pat. No. 8,246,968, herein incorporated by reference in its entirety).

In one embodiment, the therapeutic nanoparticle may comprise a multiblock copolymer (See e.g., U.S. Pat. No. 8,263,665 and 8,287,910; each of which is herein incorporated by reference in its entirety).

In one embodiment, the block copolymers described herein may be included in a polyion complex comprising a non-polymeric micelle and the block copolymer. (See e.g., U.S. Pub. No. 20120076836; herein incorporated by reference in its entirety).

In one embodiment, the therapeutic nanoparticle may comprise at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

In one embodiment, the therapeutic nanoparticles may comprise at least one cationic polymer described herein and/or known in the art.

In one embodiment, the therapeutic nanoparticles may comprise at least one amine-containing polymer such as, but not limited to polylysine, polyethylene imine, poly(amidoamine) dendrimers, poly(beta-amino esters) (See e.g., U.S. Pat. No. 8,287,849; herein incorporated by reference in its entirety) and combinations thereof.

In one embodiment, the therapeutic nanoparticles may comprise at least one degradable polyester which may contain polycationic side chains. Degradable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

In another embodiment, the therapeutic nanoparticle may include a conjugation of at least one targeting ligand. The targeting ligand may be any ligand known in the art such as, but not limited to, a monoclonal antibody. (Kirpotin et al, Cancer Res. 2006 66:6732-6740; herein incorporated by reference in its entirety).

In one embodiment, the therapeutic nanoparticle may be formulated in an aqueous solution which may be used to target cancer (see International Pub No. WO2011084513 and US Pub No. US20110294717, each of which is herein incorporated by reference in their entirety).

In one embodiment, the conjugates of the invention may be encapsulated in, linked to and/or associated with synthetic nanocarriers. Synthetic nanocarriers include, but are not limited to, those described in International Pub. Nos. WO2010005740, WO2010030763, WO201213501, WO2012149252, WO2012149255, WO2012149259, WO2012149265, WO2012149268, WO2012149282, WO2012149301, WO2012149393, WO2012149405, WO2012149411 and WO2012149454 and US Pub. Nos. US20110262491, US20100104645, US20100087337 and US20120244222, each of which is herein incorporated by reference in their entirety. The synthetic nanocarriers may be formulated using methods known in the art and/or described herein. As a non-limiting example, the synthetic nanocarriers may be formulated by the methods described in International Pub Nos. WO2010005740, WO2010030763 and WO201213501 and US Pub. Nos. US20110262491, US20100104645, US20100087337 and US20120244222, each of which is herein incorporated by reference in their entirety. In another embodiment, the synthetic nanocarrier formulations may be lyophilized by methods described in International Pub. No. WO2011072218 and U.S. Pat. No. 8,211,473; each of which is herein incorporated by reference in their entirety.

In one embodiment, the synthetic nanocarriers may contain reactive groups to release the conjugates described herein (see International Pub. No. WO20120952552 and US Pub No. US20120171229, each of which is herein incorporated by reference in their entirety).

In one embodiment, the synthetic nanocarriers may be formulated for targeted release. In one embodiment, the synthetic nanocarrier is formulated to release the conjugates at a specified pH and/or after a desired time interval. As a non-limiting example, the synthetic nanoparticle may be formulated to release the conjugates after 24 hours and/or at a pH of 4.5 (see International Pub. Nos. WO2010138193 and WO2010138194 and US Pub Nos. US20110020388 and US20110027217, each of which is herein incorporated by reference in their entirety).

In one embodiment, the synthetic nanocarriers may be formulated for controlled and/or sustained release of conjugates described herein. As a non-limiting example, the synthetic nanocarriers for sustained release may be formulated by methods known in the art, described herein and/or as described in International Pub No. WO2010138192 and US Pub No. 20100303850, each of which is herein incorporated by reference in their entirety.

In one embodiment, the nanoparticle may be optimized for oral administration. The nanoparticle may comprise at least one cationic biopolymer such as, but not limited to, chitosan or a derivative thereof. As a non-limiting example, the nanoparticle may be formulated by the methods described in U.S. Pub. No. 20120282343; herein incorporated by reference in its entirety.

### D. Polymers, Biodegradable Nanoparticles, and Core-Shell Nanoparticles

The conjugates of the invention can be formulated using natural and/or synthetic polymers. Non-limiting examples of polymers which may be used for delivery include, but are not limited to, DYNAMIC POLYCONJUGATE® (Arrowhead Research Corp., Pasadena, Calif.) formulations from MIRUS® Bio (Madison, Wis.) and Roche Madison (Madison, Wis.), PHASERX™ polymer formulations such as, without limitation, SMARTT POLYMER TECHNOLOGY™ (Seattle, Wash.), DMRI/DOPE, poloxamer, VAXFECTIN® adjuvant from Vical (San Diego, Calif.), chitosan, cyclodextrin from Calando Pharmaceuticals (Pasadena, Calif.), dendrimers and poly(lactic-co-glycolic acid) (PLGA) polymers, RONDEL™ (RNAi/Oligonucleotide Nanoparticle Delivery) polymers (Arrowhead Research Corporation, Pasadena, Calif.) and pH responsive co-block polymers such as, but not limited to, PHASERX™ (Seattle, Wash.).

A non-limiting example of chitosan formulation includes a core of positively charged chitosan and an outer portion of negatively charged substrate (U.S. Pub. No. 20120258176; herein incorporated by reference in its entirety). Chitosan includes, but is not limited to N-trimethyl chitosan, mono-N-carboxymethyl chitosan (MCC), N-palmitoyl chitosan (NPCS), EDTA-chitosan, low molecular weight chitosan, chitosan derivatives, or combinations thereof.

In one embodiment, the polymers used in the present invention have undergone processing to reduce and/or inhibit the attachment of unwanted substances such as, but not limited to, bacteria, to the surface of the polymer. The polymer may be processed by methods known and/or described in the art and/or described in International Pub. No. WO2012150467, herein incorporated by reference in its entirety.

A non-limiting example of PLGA formulations include, but are not limited to, PLGA injectable depots (e.g., ELIGARD® which is formed by dissolving PLGA in 66% N-methyl-2-pyrrolidone (NMP) and the remainder being aqueous solvent and leuprolide. Once injected, the PLGA and leuprolide peptide precipitates into the subcutaneous space).

In one embodiment, the pharmaceutical compositions may be sustained release formulations. In a further embodiment, the sustained release formulations may be for subcutaneous delivery. Sustained release formulations may include, but are not limited to, PLGA microspheres, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethicon Inc. Cornelia, Ga.), TISSELL® (Baxter International, Inc Deerfield, Ill.), PEG-based sealants, and COSEAL® (Baxter International, Inc Deerfield, Ill.).

As a non-limiting example modified mRNA may be formulated in PLGA microspheres by preparing the PLGA microspheres with tunable release rates (e.g., days and weeks) and encapsulating the conjugate in the PLGA microspheres while maintaining the integrity of the conjugate during the encapsulation process. EVAc are non-biodegradable, biocompatible polymers which are used extensively in pre-clinical sustained release implant applications (e.g., extended release products Ocusert a pilocarpine ophthalmic insert for glaucoma or progestasert a sustained release progesterone intrauterine device; transdermal delivery systems Testoderm, Duragesic and Selegiline; catheters). Poloxamer F-407 NF is a hydrophilic, non-ionic surfactant triblock copolymer of polyoxyethylene-polyoxypropylene-polyoxyethylene having a low viscosity at temperatures less than 5° C. and forms a solid gel at temperatures greater than 15° C. PEG-based surgical sealants comprise two synthetic PEG components mixed in a delivery device which can be prepared in one minute, seals in 3 minutes and is reabsorbed within 30 days. GELSITE® and natural polymers are capable of in-situ gelation at the site of administration. They have been shown to interact with protein and peptide therapeutic candidates through ionic interaction to provide a stabilizing effect.

Polymer formulations can also be selectively targeted through expression of different ligands as exemplified by, but not limited by, folate, transferrin, and N-acetylgalactosamine (GalNAc) (Benoit et al., Biomacromolecules. 2011 12:2708-2714; Rozema et al., Proc Natl Acad Sci USA. 2007 104:12982-12887; Davis, Mol Pharm. 2009, 6:659-668; Davis, Nature, 2010, 464:1067-1070; each of which is herein incorporated by reference in its entirety).

The conjugates of the invention may be formulated with or in a polymeric compound. The polymer may include at least one polymer such as, but not limited to, polyethenes, polyethylene glycol (PEG), poly(l-lysine)(PLL), PEG grafted to PLL, cationic lipopolymer, biodegradable cationic lipopolymer, polyethylenimine (PEI), cross-linked branched poly(alkylene imines), a polyamine derivative, a modified poloxamer, a biodegradable polymer, elastic biodegradable polymer, biodegradable block copolymer, biodegradable random copolymer, biodegradable polyester copolymer, biodegradable polyester block copolymer, biodegradable polyester block random copolymer, multiblock copolymers, linear biodegradable copolymer, poly[α-(4-aminobutyl)-L-glycolic acid) (PAGA), biodegradable cross-linked cationic multi-block copolymers, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester), acrylic polymers, amine-containing polymers, dextran polymers, dextran polymer derivatives or combinations thereof.

As a non-limiting example, the conjugates of the invention may be formulated with the polymeric compound of PEG grafted with PLL as described in U.S. Pat. No. 6,177,274; herein incorporated by reference in its entirety. In another example, the conjugate may be suspended in a solution or medium with a cationic polymer, in a dry pharmaceutical composition or in a solution that is capable of being dried as described in U.S. Pub. Nos. 20090042829 and 20090042825; each of which are herein incorporated by reference in their entireties.

As another non-limiting example the conjugate of the invention may be formulated with a PLGA-PEG block copolymer (see US Pub. No. US20120004293 and U.S. Pat. No. 8,236,330, each of which are herein incorporated by reference in their entireties) or PLGA-PEG-PLGA block copolymers (See U.S. Pat. No. 6,004,573, herein incorporated by reference in its entirety). As a non-limiting example, the conjugate of the invention may be formulated with a diblock copolymer of PEG and PLA or PEG and PLGA (see U.S. Pat. No 8,246,968, herein incorporated by reference in its entirety).

A polyamine derivative may be used to deliver conjugates of the invention or to treat and/or prevent a disease or to be included in an implantable or injectable device (U.S. Pub. No. 20100260817 herein incorporated by reference in its entirety). As a non-limiting example, a pharmaceutical composition may include the conjugates of the invention and the polyamine derivative described in U.S. Pub. No. 20100260817 (the contents of which are incorporated herein by reference in its entirety). As a non-limiting example the conjugates of the invention may be delivered using a polyamide polymer such as, but not limited to, a polymer comprising a 1,3-dipolar addition polymer prepared by combining a carbohydrate diazide monomer with a dilkyne unite comprising oligoamines (U.S. Pat. No. 8,236,280; herein incorporated by reference in its entirety).

The conjugate of the invention may be formulated with at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

In one embodiment, the conjugates of the invention may be formulated with at least one polymer and/or derivatives thereof described in International Publication Nos. WO2011115862, WO2012082574 and WO2012068187 and U.S. Pub. No. 20120283427, each of which are herein incorporated by reference in their entireties. In another embodiment, the conjugates of the invention may be formulated with a polymer of formula Z as described in WO2011115862, herein incorporated by reference in its entirety. In yet another embodiment, the conjugates of the invention may be formulated with a polymer of formula Z, Z′ or Z″ as described in International Pub. Nos. WO2012082574 or WO2012068187, each of which are herein incorporated by reference in their entireties. The polymers formulated with the conjugates of the present invention may be synthesized by the methods described in International Pub. Nos. WO2012082574 or WO2012068187, each of which are herein incorporated by reference in their entireties.

Formulations of conjugates of the invention may include at least one amine-containing polymer such as, but not limited to polylysine, polyethylene imine, poly(amidoamine) dendrimers or combinations thereof.

For example, the conjugate of the invention may be formulated in a pharmaceutical compound including a poly(alkylene imine), a biodegradable cationic lipopolymer, a biodegradable block copolymer, a biodegradable polymer, or a biodegradable random copolymer, a biodegradable polyester block copolymer, a biodegradable polyester polymer, a biodegradable polyester random copolymer, a linear biodegradable copolymer, PAGA, a biodegradable cross-linked cationic multi-block copolymer or combinations thereof. The biodegradable cationic lipopolymer may be made by methods known in the art and/or described in U.S. Pat. No. 6,696,038, U.S. App. Nos. 20030073619 and 20040142474 each of which is herein incorporated by reference in their entireties. The poly(alkylene imine) may be made using methods known in the art and/or as described in U.S. Pub. No. 20100004315, herein incorporated by reference in its entirety. The biodegradable polymer, biodegradable block copolymer, the biodegradable random copolymer, biodegradable polyester block copolymer, biodegradable polyester polymer, or biodegradable polyester random copolymer may be made using methods known in the art and/or as described in U.S. Pat. Nos. 6,517,869 and 6,267,987, the contents of which are each incorporated herein by reference in their entirety. The linear biodegradable copolymer may be made using methods known in the art and/or as described in U.S. Pat. No. 6,652,886. The PAGA polymer may be made using methods known in the art and/or as described in U.S. Pat. No. 6,217,912 herein incorporated by reference in its entirety. The PAGA polymer may be copolymerized to form a copolymer or block copolymer with polymers such as but not limited to, poly-L-lysine, polyarginine, polyornithine, histones, avidin, protamines, polylactides and poly(lactide-co-glycolides). The biodegradable cross-linked cationic multi-block copolymers may be made my methods known in the art and/or as described in U.S. Pat. No. 8,057,821 or U.S. Pub. No. 2012009145 each of which are herein incorporated by reference in their entireties. For example, the multi-block copolymers may be synthesized using linear polyethylenimine (LPEI) blocks which have distinct patterns as compared to branched polyethyleneimines. Further, the composition or pharmaceutical composition may be made by the methods known in the art, described herein, or as described in U.S. Pub. No. 20100004315 or U.S. Pat. Nos. 6,267,987 and 6,217,912 each of which are herein incorporated by reference in their entireties.

The conjugates of the invention may be formulated with at least one degradable polyester which may contain polycationic side chains. Degradable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

The conjugate of the invention may be formulated with at least one cross linkable polyester. Cross linkable polyesters include those known in the art and described in US Pub. No. 20120269761, herein incorporated by reference in its entirety.

In one embodiment, the polymers described herein may be conjugated to a lipid-terminating PEG. As a non-limiting example, PLGA may be conjugated to a lipid-terminating PEG forming PLGA-DSPE-PEG. As another non-limiting example, PEG conjugates for use with the present invention are described in International Publication No. WO2008103276, herein incorporated by reference in its entirety. The polymers may be conjugated using a ligand conjugate such as, but not limited to, the conjugates described in U.S. Pat. No. 8,273,363, herein incorporated by reference in its entirety.

In one embodiment, the conjugates of the invention may be conjugated with another compound. Non-limiting examples of conjugates are described in U.S. Pat. Nos. 7,964,578 and 7,833,992, each of which are herein incorporated by reference in their entireties. In another embodiment, the conjugates of the invention may be conjugated with conjugates of formula 1-122 as described in U.S. Pat. Nos. 7,964,578 and 7,833,992, each of which are herein incorporated by reference in their entireties. The modified RNA described herein may be conjugated with a metal such as, but not limited to, gold. (See e.g., Giljohann et al. Journ. Amer. Chem. Soc. 2009 131(6): 2072-2073; herein incorporated by reference in its entirety). In another embodiment, the conjugates of the invention may be conjugated and/or encapsulated in gold-nanoparticles. (International Pub. No. WO201216269 and U.S. Pub. No. 20120302940; each of which is herein incorporated by reference in its entirety).

In one embodiment, the polymer formulation of the present invention may be stabilized by contacting the polymer formulation, which may include a cationic carrier, with a cationic lipopolymer which may be covalently linked to cholesterol and polyethylene glycol groups. The polymer formulation may be contacted with a cationic lipopolymer using the methods described in U.S. Pub. No. 20090042829 herein incorporated by reference in its entirety. The cationic carrier may include, but is not limited to, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane (DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-propanaminium trifluoroacetate (DOSPA), 3B-[N(N′,N′-Dimethylaminoethane)-carbamoyl]Cholesterol Hydrochloride (DC-Cholesterol HCl) diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride DODAC) and combinations thereof.

The conjugates of the invention may be formulated in a polyplex of one or more polymers (U.S. Pub. No. 20120237565 and 20120270927; each of which is herein incorporated by reference in its entirety). In one embodiment, the polyplex comprises two or more cationic polymers. The catioinic polymer may comprise a poly(ethylene imine) (PEI) such as linear PEI.

The conjugates of the invention can also be formulated as a nanoparticle using a combination of polymers, lipids, and/or other biodegradable agents, such as, but not limited to, calcium phosphate. Components may be combined in a core-shell, hybrid, and/or layer-by-layer architecture, to allow for fine-tuning of the nanoparticle so that delivery of the conjugates of the invention may be enhanced (Wang et al., Nat Mater. 2006, 5:791-796; Fuller et al., Biomaterials. 2008, 29:1526-1532; DeKoker et al., Adv Drug Deliv Rev. 2011, 63:748-761; Endres et al., Biomaterials. 2011, 32:7721-7731; Su et al., Mol Pharm. 2011, Jun. 6;8(3):774-87; each of which is herein incorporated by reference in its entirety). As a non-limiting example, the nanoparticle may comprise a plurality of polymers such as, but not limited to hydrophilic-hydrophobic polymers (e.g., PEG-PLGA), hydrophobic polymers (e.g., PEG) and/or hydrophilic polymers (International Pub. No. WO20120225129; herein incorporated by reference in its entirety).

Biodegradable calcium phosphate nanoparticles in combination with lipids and/or polymers have been shown to deliver therapeutic agents in vivo. In one embodiment, a lipid coated calcium phosphate nanoparticle, which may also contain a targeting ligand such as anisamide, may be used to deliver the conjugate of the present invention. For example, to effectively deliver a therapeutic agent in a mouse metastatic lung model a lipid coated calcium phosphate nanoparticle was used (Li et al., J Contr Rel. 2010, 142: 416-421; Li et al., J Contr Rel. 2012, 158:108-114; Yang et al., Mol Ther. 2012, 20:609-615; herein incorporated by reference in its entirety). This delivery system combines both a targeted nanoparticle and a component to enhance the endosomal escape, calcium phosphate, in order to improve delivery of the therapeutic agent.

In one embodiment, a PEG-charge-conversional polymer (Pitella et al., Biomaterials. 2011, 32:3106-3114) may be used to form a nanoparticle to deliver the conjugate of the present invention. The PEG-charge-conversional polymer may improve upon the PEG-polyanion block copolymers by being cleaved into a polycation at acidic pH, thus enhancing endosomal escape.

The use of core-shell nanoparticles has additionally focused on a high-throughput approach to synthesize cationic cross-linked nanogel cores and various shells (Siegwart et al., Proc Natl Acad Sci USA. 2011, 108:12996-13001). The complexation, delivery, and internalization of the polymeric nanoparticles can be precisely controlled by altering the chemical composition in both the core and shell components of the nanoparticle. For example, the core-shell nanoparticles may efficiently deliver a therapeutic agent to mouse hepatocytes after they covalently attach cholesterol to the nanoparticle.

The use of core-shell nanoparticles has additionally focused on a high-throughput approach to synthesize cationic cross-linked nanogel cores and various shells (Siegwart et al., Proc Natl Acad Sci USA. 2011, 108:12996-13001). The complexation, delivery, and internalization of the polymeric nanoparticles can be precisely controlled by altering the chemical composition in both the core and shell components of the nanoparticle. For example, the core-shell nanoparticles may efficiently deliver a therapeutic agent to mouse hepatocytes after they covalently attach cholesterol to the nanoparticle.

In one embodiment, the lipid nanoparticles may comprise a core of the conjugates disclosed herein and a polymer shell. The polymer shell may be any of the polymers described herein and are known in the art. In an additional embodiment, the polymer shell may be used to protect the modified nucleic acids in the core.

Core-shell nanoparticles for use with the conjugates of the present invention are described and may be formed by the methods described in U.S. Pat. No. 8,313,777 herein incorporated by reference in its entirety.

In one embodiment, the core-shell nanoparticles may comprise a core of the conjugates disclosed herein and a polymer shell. The polymer shell may be any of the polymers described herein and are known in the art. In an additional embodiment, the polymer shell may be used to protect the modified nucleic acid molecules in the core.

### E. Inorganic Nanoparticles

Inorganic nanoparticles exhibit a combination of physical, chemical, optical and electronic properties and provide a highly multifunctional platform to image and diagnose diseases, to selectively deliver therapeutic agens, and to sensitive cells and tissues to treatment regiments. Not wishing to be bound to any theory, enhanced permeability and retention (EPR) effect provides a basis for the selective accumulation of many high-molecular-weight drugs. Circulating inorganic nanoparticles preferentially accumulate at tumor sites and in inflamed tissues (Yuan et al., Cancer Res., vol. 55(17):3752-6, 1995, the contents of which are incorporated herein by reference in their entirety) and remain lodged due to their low diffusivity (Pluen et al., PNAS, vol.98(8):4628-4633, 2001, the contents of which are incorporated herein by reference in their entirety). The size of the inorganic nanoparticles may be 10 nm-500 nm, 10 nm-100 nm or 100 nm-500 nm. The inorganic nanoparticles may comprise metal (gold, iron, silver, copper, nickel, etc.), oxides (ZnO, TiO₂, Al₂O₃, SiO₂, iron oxide, copper oxide, nickel oxide, etc.), or semiconductor (CdS, CdSe, etc.). The inorganic nanoparticles may also be perfluorocarbon or FeCo.

Inorganic nanoparticles have high surface area per unit volume. Therefore, they may be loaded with therapeutic drugs and imaging agents at high densitives. A variety of methods may be used to load therapeutic drugs into/onto the inorganic nanoparticles, including but not limited to, colvalent bonds, electrostatic interactions, entrapment, and encapsulation. In addition to therapeutic agent drug loads, the inorganic nanoparticles may be funcationalized with targeting moieties, such as tumor-targeting ligands, on the surface. Formulating therapeutic agents with inorganic nanoparticles allows imaging, detection and monitoring of the therapeutic agents.

In one embodiment, the conjugate of the invention is hydrophobic and may be form a kinetically stable complex with gold nanoparticles funcationalized with water-soluble zwitterionic ligands disclosed by Kim et al. (Kim et al., JACS, vol. 131(4):1360-1361, 2009, the contents of which are incorporated herein by reference in their entirety). Kim et al. demonstrated that hydrophobic drugs carried by the gold nanoparticles are efficiently released into cells with little or no cellular uptake of the gold nanoparticles.

In one embodiment, the conjugates of the invention may be formulated with gold nanoshells. As a non-limiting example, the conjugates may be delivered with a temperature sensitive system comprising polymers and gold nanoshells and may be released photothermally. Sershen et al. designed a delivery vehicle comprising hydrogel and gold nanoshells, wherein the hydrogels are made of copolymers of N-isopropylacrylamide (NIPAAm) and acrylamide (AAm) and the gold nanoshells are made of gold and gold sulfide (Sershen et al., J Biomed Mater, vol. 51:293-8, 2000, the contents of which are incorporated herein by reference in their entirety). Irradiation at 1064 nm was absorbed by the nanoshells and converted to heat, which led to the collapse of the hydrogen and release of the drug. The conjugate of the invention may also be encapsulated inside hollow gold nanoshells.

In some embodiments, the conjugates of the invention may be attached to gold nanoparticles via covalent bonds. Covalent attachment to gold nanoparticles may be achieved through a linker, such as a free thiol, amine or carboxylate functional group. In some embodiments, the linkers are located on the surface of the gold nanoparticles. In some embodiments, the conjugates of the invention may be modified to comprise the linkers. The linkers may comprise a PEG or oligoethylene glycol moiety with varying length to increase the particles' stability in biological environment and to control the density of the drug loads. PEG or oligoethylene glycol moieties also minimize nonspecific adsorption of undesired biomolecules. PEG or oligoethylene gycol moieties may be branched or linear. Tong et al. disclosed that branched PEG moieties on the surface of gold nanoparticles increase circulatory half-life of the gold nanoparticles and reduced serum protein binding (Tong et al., Langmuir, vol. 25(21):12454-9, 2009, the contents of which are incorporated herein by reference in their entirety).

In one embodiment, the conjugate of the invention may comprise PEG-thiol groups and may attach to gold nanoparticles via the thiol group. The synthesis of thiol-PEGylated conjugates and the attachment to gold nanoparticles may follow the method disclosed by El-Sayed et al. (El-Sayed et al., Bioconjug. Chem., vol. 20(12):2247-2253, 2010, the contents of which are incorporated herein by reference in their entirety).

In another embodiment, the conjugate of the invention may be tethered to an amine-functionalized gold nanoparticles. Lippard et al. disclosed that Pt(IV) prodrugs may be delivered with amine-functionalized polyvalent oligonucleotide gold nanoparticles and are only activated into their active Pt(II) forms after crossing the cell membrane and undergoing intracellular reduction (Lippard et al., JACS, vol. 131(41):14652-14653, 2009, the contents of which are incorporated herein by reference in their entirety). The cytotoxic effects for the Pt(IV)-gold nanoparticle complex are higher than the free Pt(IV) drugs and free cisplatin.

In some embodiments, conjugates of the invention are formulated with magnetic nanoparticle such as iron, cobalt, nickel and oxides thereof, or iron hydroxide nanoparticles. Localized magnetic field gradients may be used to attract magnetic nanoparticles to a chosen site, to hold them until the therapy is complete, and then to remove them. Magnetic nanoparticles may also be heated by magnetic fields. Alexiou et al. prepared an injection of magnetic particle, Ferro fluids (FFs), bound to anticancer agents and then concentrated the particles in the desired tumor area by an external magnetic field (Alexiou et al., Cancer Res. vol. 60(23):6641-6648, 2000, the contents of which are incorporated herein by reference in their entirety). The desorption of the anticancer agent took place within 60 min to make sure that the drug can act freely once localized to the tumor by the magnetic field.

In some embodiments, the conjugates of the invention are loaded onto iron oxide nanoparticles. In some embodiments, the conjugates of the invention are formulated with super paramagnetic nanoparticles based on a core consisting of iron oxides (SPION). SPION are coated with inorganic materials (silica, gold, etc.) or organic materials (phospholipids, fatty acids, polysaccharides, peptides or other surfactants and polymers) and can be further functionalized with drugs, proteins or plasmids.

In one embodiment, water-dispersible oleic acid (OA)-poloxamer-coated iron oxide magnetic nanoparticles disclosed by Jain et al. (Jain, Mol. Pharm., vol. 2(3):194-205, 2005, the contents of which are incorporated herein by reference in their entirety) may be used to deliver the conjugates of the invention. Therapeutic drugs partition into the OA shell surrounding the iron oxide nanoparticles and the poloxamer copolymers (i.e., Pluronics) confers aqueous dispersity to the formulation. According to Jain et al., neither the formulation components nor the drug loading affected the magnetic properties of the core iron oxide nanoparticles. Sustained release of the therapeutic drugs was achieved.

In one embodiment, the conjugates of the invention are bonded to magnetic nanoparticles with a linker. The linker may be a linker capable of undergoing an intramolecular cyclization to release the conjugates of the invention. Any linker and nanoparticles disclosed in WO2014124329 to Knipp et al., the contents of which are incorporated herein by reference in their entirety, may be used. The cyclization may be induced by heating the magnetic nanoparticle or by application of an alternating electromagnetic field to the magnetic nanoparticle.

In one embodiment, the conjugates of the invention may be delivered with a drug delivery system disclosed in U.S. Pat. No. 7,329,638 to Yang et al., the contents of which are incorporated herein by reference in their entirety. The drug delivery system comprises a magnetic nanoparticle associated with a positively charged cationic molecule, at least one therapeutic agent and a molecular recognition element.

In one embodiment, nanoparticles having a phosphate moiety are used to deliver the conjugates of the invention. The phosphate-containing nanoparticle disclosed in U.S. Pat. No. 8,828,975 to Hwu et al., the contents of which are incorporated herein by reference in their entirety, may be used. The nanoparticles may comprise gold, iron oxide, titanium dioxide, zinc oxide, tin dioxide, copper, aluminum, cadmium selenide, silicon dioxide or diamond. The nanoparticles may contain a PEG moiety on the surface.

### F. Peptides and Proteins

The conjugate of the invention can be formulated with peptides and/or proteins in order to increase peneration of cells by the conjugates of the invention. In one embodiment, peptides such as, but not limited to, cell penetrating peptides and proteins and peptides that enable intracellular delivery may be used to deliver pharmaceutical formulations. A non-limiting example of a cell penetrating peptide which may be used with the pharmaceutical formulations of the present invention include a cell-penetrating peptide sequence attached to polycations that facilitates delivery to the intracellular space, e.g., HIV-derived TAT peptide, penetratins, transportans, or hCT derived cell-penetrating peptides (see, e.g., Caron et al., Mol. Ther. 3(3):310-8 (2001); Langel, Cell-Penetrating Peptides: Processes and Applications (CRC Press, Boca Raton Fla., 2002); El-Andaloussi et al., Curr. Pharm. Des. 2003, 11(28):3597-611; and Deshayes et al., Cell. Mol. Life Sci. 2005, 62(16):1839-49, all of which are incorporated herein by reference). The compositions can also be formulated to include a cell penetrating agent, e.g., liposomes, which enhance delivery of the compositions to the intracellular space. The conjugates of the invention may be complexed to peptides and/or proteins such as, but not limited to, peptides and/or proteins from Aileron Therapeutics (Cambridge, Mass.) and Permeon Biologics (Cambridge, Mass.) in order to enable intracellular delivery (Cronican et al., ACS Chem. Biol. 2010, 5:747-752; McNaughton et al., Proc. Natl. Acad. Sci. USA 2009, 106:6111-6116; Sawyer, Chem Biol Drug Des. 2009, 73:3-6; Verdine and Hilinski, Methods Enzymol. 2012, 503:3-33; all of which are herein incorporated by reference in its entirety). In one embodiment, the cell-penetrating polypeptide may comprise a first domain and a second domain. The first domain may comprise a supercharged polypeptide. The second domain may comprise a protein-binding partner. As used herein, “protein-binding partner” includes, but are not limited to, antibodies and functional fragments thereof, scaffold proteins, or peptides. The cell-penetrating polypeptide may further comprise an intracellular binding partner for the protein-binding partner. The cell-penetrating polypeptide may be capable of being secreted from a cell where conjugates of the invention may be introduced.

### G. Vaccines

In some embodiments of the present invention, compositions of the present invention may be formulated as vaccines, such as cancer vaccines. Cancer vaccines aim to augment immune responses with the tumor antigen-expressing targets already present, such as by inducing antigen specific T cells. The general composition of cancer vaccines may include a source of TAAs and adjuvants that results in activation of dendritic cells for productive antigen presentation. The adjuvants may be oil-based formulations, Toll like receptor (TLR) ligands, recombinant cytokines or the natural innate ligands.

Adjuvants may be aluminium based adjuvants including but not limiting to aluminium hydroxide and aluminium phosphate; saponins such as steroid saponins and triterpenold saponins; bacterial flagellin and some cytokines such as GM-CSF. Adjuvants selection may depend on antigens, vaccines and routes of administrations.

Adjuvants may include, but are not limited to, alpha glucose bearing glycosphingolipid compounds disclosed by Chen et al (US Patent publication NO. 2015/0071960, the content of which is incorporated herein by reference in its entirety). Those compounds when added into the present particles in combination with conjugates of the present invention, can elevate invariant natural killer T (iNKT) cells and increases cytokine and/or chemokine production, where the cytokine production is sufficient to transactivate downstream immune cells including dendritic cells, natural killer cells, B cells, CD+4 T and CD8+ T cells.

In some embodiments, adjuvants improve the adaptive immune response to a vaccine antigen by modulating innate immunity or facilitating transport and presentation. Adjuvants act directly or indirectly on antigen presenting cells (APCs) including dendritic cells (DCs). Adjuvants may be ligands for toll-like receptors (TLRs) and can directly affect DCs to alter the strength, potency, speed, duration, bias, breadth, and scope of adaptive immunity. In other instances, adjuvants may signal via proinflammatory pathways and promote immune cell infiltration, antigen presentation, and effector cell maturation. This class of adjuvants includes mineral salts, oil emulsions, nanoparticles, and polyelectrolytes and comprises colloids and molecular assemblies exhibiting complex, heterogeneous structures (Powell et al., Clin Exp. Vaccine Res., Polyionic vaccine adjuvants: another look at aluminum salts and polyelectrolytes. 2015, 4(1):23-45).

In one example, heat shock proteins or their peptide derivatives may be used as an adjuvant in the composition as disclosed in Shevtsov et al. (Frontiers in Immunology, vol. 7:article 171 (2016)). For example, HSP70 protein, HSP70 peptide derived thereof, HSP90 protein, HSP90 peptide derived thereof may be combined with conjuates or particles of the present invention to produce vaccine compositions.

In another example, the vaccine composition comprises conjugates or particles of the present invention and synthetic toll like receptor-4 (TLR-4) agonist peptides disclosed in Shanmugam et al. (PloS ONE, vol. 7(2):e30839 (2012)) as adjuvants.

In yet another example, the vaccine composition comprises conjugates or particles of the present invention and pidotimod as an adjuvant.

In some embodiments, conjugates of the present invention may be formulated as peptide based vaccines. Such vaccines may target directly to dendritic cells in vivo to activate dendritic cells for presenting antigens. Conjugates may be formulated as micro- or nanoparticles, liposomes and/or virus-like particles (VLP) to increase intracellular membrane permeability. In some embodiments, nanoparticle cancer vaccines may be formulated to deliver several TAAs and adjuvants simultaneously, enabling a coordinated activation of dendritic cells. In other embodiments, nanoparticles can also be functionalized in order to actively target dendritic cells in vivo, to increase their cellular internalization and immunogenicity or even target specific intracellular compartments (Silva, J M., et al., J. Control. Release 2013, 168:179-199; the contents of which are incorporated herein by reference in their entirety).

In some embodiments, nanoparticle formulations as described may be modified for imaging, diagnostic, and targeted delivery of conjugates to immune cells.

In one embodiment, the conjugate of the present invention may be delivered with a liposomal drug delivery system as reported by van Broekhoven, C L., et al., Cancer Res. 2004, 12:4357-65; the contents of which are incorporated herein by reference in their entirety, which targets dendritic cells as a platform to induce a highly effective immunity against tumor cells. Studies of liposome-DNA complexes have also been described, constituting an effective strategy to elicit anti-tumor immunity (U'Ren, L., et al., Cancer Gene Ther. 2006, 11:1033-44; the contents of which are incorporated herein by reference in their entirety).

In one embodiment, the conjugate of the present invention may be formulated into self-assembling spherical polymeric micelles formed by amphiphilic block copolymers in an aqueous medium. A hydrophobic core and a hydrophilic surface compose these structures and their size ranges from 10 to 100 nm (Torchilin, V P., J. Control. Release 2001, 73(2-3):137-72; the contents of which are incorporated herein by reference in their entirety). In another embodiment, novel pH-responsive polymer micelles formed by an N-(2-hydroxypropyl) methacrylamide corona and a propylacrylic acid (PAA)/dimethylaminoethyl methacrylate (DMAEMA)/butyl methacrylate (BMA) core have been investigated for antigen trafficking modulation in dendritic cells. The results showed that this nanosystem facilitates antigen delivery to dendritic cells in the lymph nodes and enhances CD8+ T cell responses, being thus a potential carrier for cancer vaccines. Keller S., et al., J. Control. Release 2014, 191:24-33; the contents of which are incorporated herein by reference in their entirety. In another embodiment, micelles formed by DMAEMA and pyridyl disulfide ethyl methacrylate (PDSEMA), carrying both short single-stranded synthetic DNA molecules which contain a cytosine triphosphate deoxynucleotide followed by a guanine triphosphate deoxynucleotide (CpG ODN) and protein antigens, have shown to elicit and increase the cellular and humoral immune response by modulating and stimulating antigen cross-presentation, as summarized by Wilson J T., et al., ACS Nano. 2013, 7(5):3912-25; the contents of which are incorporated herein by reference in their entirety.

In another embodiment, polymers from different origins already described as useful materials for polymeric nanoparticle production and used in other preclinical studies may be formulated with the conjugate of the present invention. Polymers can be from natural origin, such as chitosan, or synthesized, as polylactic acid and poly-lactic-co-glycolic acid (PLGA). Particulate adjuvants, such as PLGA and polycaprolactones (PCL) nanoparticles, have generated a lot of interest due to their biodegradability, biocompatibility and mechanical strength. Danhier F., et al., J. Control. Release 2012, 161(2):505-22; the contents of which are incorporated herein by reference in their entirety, characterizes these adjuvant nanoparticles as maintaining the antigenicity and immunogenicity of their encapsulated proteins. PLGA has been used for decades in humans and is the most studied polymer for vaccine formulations and has shown to increase antibody and cellular responses to antigen-loaded PLGA nanoparticle. (Johansen, P., et al., Vaccine. 2000, 19(9-10):1047-54; Shen, H., et al., Immunology. 2006, 117(1):78-88; Chen, M. et al., Cell Immunol. 2014, 287(2):91-9; the contents of each of which are incorporated herein by reference in their entirety). Further, PCL has the characteristics of an antigen controlled release matrices by its low degradation rate, hydrophobicity, good drug permeability, in vitro stability and low toxicity. The adjuvant effect of PCL nanoparticles to induce immune responses against an infectious disease was previously confirmed by several studies (Benoit, M A., et al., Int. J. Pharm. 1999, 184(1):73-84; Florindo, H F., et al., Vaccine. 2008, 26(33):4168-77; Florindo, H F., et al., Biomaterials. 2009, 30(5):879-91; Labet, M., et al., Chem Soc Rev. 2009, 38(12):3484-504; the contents of each of which are incorporated herein by reference in their entirety). If the encapsulated antigen fails to induce dendritic cell activation, these nanoparticles may be modified with maturation signals at their surface for direct ligand-receptor interaction, as mannose receptors are overexpressed at dendritic cell and macrophage cell surfaces.

In another embodiment, the conjugate of the present invention may be loaded into PLGA nanoparticles with melanoma antigens to elicit effective anti-tumor activity by CTLs in vivo (Zhang, Z., et al., Biomaterials. 2011, 32(14):3666-78; Ma, W., et al., Int. J. Nanomedicine. 2012, 7:1475-87; the contents of each of which are incorporated herein by reference in their entirety). In addition, chitosan nanoparticles targeting dendritic cells carrying IL-12 were administered in an animal model that resulted in suppression of tumor growth and increased induction of apoptosis (Kim, T H., et al., Mol Cancer Ther. 2006, 5(7):1723-32; the contents of which are incorporated herein by reference in their entirety).

In another embodiment, the conjugate of the present invention may be delivered with a hyper-branched spherical dendrimer nanocarrier with a hydrophilic surface and a hydrophobic central core. (Lee, C C., et al., Nat Biotechnol. 2005, 23(12):1517-26; the contents of which are incorporated herein by reference in their entirety). The linear poly(glutamic acid) is a poly(amino acid) polymer has been reported to have considerable potential for antigen delivery to dendritic cells, adjuvant properties for dendritic cell maturation, and able to induce CTLs (Yoshikawa, T., et al., Vaccine. 2008, 26(10):1303-13; the contents of which are incorporated herein by reference in their entirety).

### IV. Administration, Dose and Dosage Form

Administration: Compositions and formulations containing an effective amount of conjugates or particles of the present invention may be administered to a subject in need thereof by any route which results in a therapeutically effective outcome in said subject. These include, but are not limited to enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), intramyocardial (entering the myocardium), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal. In specific embodiments, compositions may be administered in a way which allows them cross the blood-brain barrier, vascular barrier, or other epithelial barrier.

In some embodiments, particles, nanoparticles and/or polymeric nanoparticles are administered to bone marrow. In some embodiments, particles, nanoparticles and/or polymeric nanoparticles are administered to areas having a lot of dendritic cells, such as subcutaneous space.

Dose and Dosage forms: Compositions in accordance with the invention are typically formulated in dosage unit form for ease of administration and uniformity of dosage. It will be understood, however, that the total daily usage of the compositions of the present invention may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, compositions in accordance with the present invention may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect. The desired dosage may be delivered three times a day, two times a day, once a day, every other day, every third day, every week, every two weeks, every three weeks, or every four weeks. In some embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used.

As used herein, a “split dose” is the division of single unit dose or total daily dose into two or more doses, e.g., two or more administrations of the single unit dose. As used herein, a “single unit dose” is a dose of any therapeutic administed in one dose/at one time/single route/single point of contact, i.e., single administration event. As used herein, a “total daily dose” is an amount given or prescribed in 24 hr. period. It may be administered as a single unit dose.

A pharmaceutical composition described herein can be formulated into a dosage form described herein, such as a topical, intranasal, intratracheal, or injectable (e.g., intravenous, intraocular, intravitreal, intramuscular, intracardiac, intraperitoneal, and subcutaneous).

In some embodiments, the dosage forms may be liquid dosage forms. Liquid dosage forms for parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art including, but not limited to, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. In certain embodiments for parenteral administration, compositions may be mixed with solubilizing agents such as CREMOPHOR®, alcohols, oils, modified oils, glycols, polysorbates, cyclodextrins, polymers, and/or combinations thereof.

In certain embodiments, the dosages forms may be injectable. Injectable preparations, for example, sterile injectable aqueous or oleaginous suspensions may be formulated according to the known art and may include suitable dispersing agents, wetting agents, and/or suspending agents. Sterile injectable preparations may be sterile injectable solutions, suspensions, and/or emulsions in nontoxic parenterally acceptable diluents and/or solvents, for example, a solution in 1,3-butanediol. Among the acceptable vehicles and solvents that may be employed include, but are not limited to, water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables. Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it may be desirable to slow the absorption of the active ingredient from subcutaneous or intramuscular injection. This may be accomplished by the use of a liquid suspension of crystalline or amorphous material with poor water solubility. The rate of absorption of the compounds then depends upon its rate of dissolution which, in turn, may depend upon crystal size and crystalline form. Injectable depot forms are made by forming microencapsule matrices of the conjugates in biodegradable polymers such as polylactide-polyglycolide. Depending upon the ratio of conjugates to polymer and the nature of the particular polymer employed, the rate of active agents in the conjugates can be controlled. Examples of other biodegradable polymers include, but are not limited to, poly(orthoesters) and poly(anhydrides). Depot injectable formulations may be prepared by entrapping the conjugates in liposomes or microemulsions which are compatible with body tissues.

In some embodiments, solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

### Immuno-Oncology Therapies

Compositions of the present invention may be used to harness the immune system to eliminate tumor cells. In some embodiments, compositions of the present invention may be used as vaccines. Vaccines may be peptide vaccines such as conjugates, particles comprising conjugates of TAAs, TAA epitopes or derivatives thereof; or dendritic cell vaccines to increase the frequency of tumor specific cytotoxic T lymphocytes, wherein the DCs are primed with conjugates and/or particles comprising conjugates of TAAs, TAA epitopes or derivatives thereof; or adoptive cellular immunotherapies involving adoptive transfer of effector T cells which are actived by compositions of the present inventions.

In accordance with the present inventions, compositions as discussed above may be used either for active immunotherapy and adoptive immunotherapy to prevent/treat a disease such as cancer. In an active immunotherapy, compositions of the present invention may be used to prime and amplify Tumor antigen specific T cells in vivo. For an adoptive immunotherapy, T cells are isolated from a subject to be treated and may be primed and amplified using compositions of the present invention ex vivo prior to their infusion. Adoptive immunotherapy is a procedure whereby an individual's own T cells are expanded ex vivo and re-infused back into the body. In one embodiment, particles of the present invention may facilitate the delivery of the T cells that have been activated ex vivo. Both active and adoptive immunotherapy can be used as therapeutic strategies for treatment of cancer.

In other embodiments, compositions of the present invention may be used for antibody-based cancer immunotherapy. Conjugates comprising antibodies or derivatives thereof may be used for this therapy. In other embodiments, compositions of the present invention may be used for cytokine based cancer immunotherapy.

### A. Tumor Antigen Peptide Vaccines

In accordance with the present invention, conjugates comprising one or more tumor antigenic peptides, and/or particles and formulations comprising such conjugates may be used as peptide vaccines to treat a tumor. Peptide vaccines may be used to induce antibodies that can react with the native protein expressed in tumor cells and promote complement-mediated lysis of tumor cells, or elicit T cell based cellular immune response to destroy tumor cells, or both.

Classically, tumor antigens of interest to cancer vaccine development have been categorized into “unique or neoantigens,” which are unique to a tumor tissue and are not present in normal tissue and “shared antigens” which are common among two or more tumors or populations.

Shared antigens can fall into several categories. The first category is composed of certain shared tumor associated antigens derived from proteins that are expressed in cancer but not expressed in most normal tissues. This category includes the cancer/testis (CT) antigens, which are expressed in certain tumors, but in normal tissue are found only in placental trophoblasts and testicular germ cells. Alternatively, a tumor antigen may be an antigen that is specific to the tissue in which the tumor arises. Because of their limited tissue distribution, the CT antigens or lineage/tissue specific antigens may not cause “off target” immune response-related toxicities, even if high-affinity T cells are elicited upon antigen vaccination. The third group under consideration is consists of certain tumor associated antigens, which arise from genes which are overexpressed in certain tumors, but are also expressed in normal tissues, albeit at lower levels. This group is more precarious, since an immunotherapeutic approach targeting these antigens may result in side effects in the normal tissues if a certain threshold of response is overstepped.

Shared antigens have the benefit of being present on many tumors of a certain type, which allows a broader applicability of the tumor antigen as a vaccine component and therefore scalability of the vaccination approach. However, cancer vaccines which have used self-antigens that are selectively expressed or overexpressed in tumors have failed to elicit therapeutic immunity and had disappointing clinical outcomes (as reviewed in Platten and Ofringa, Cell Research (2015) 25:887-888). This may be due to the fact that these self-antigens must overcome central tolerance, which normally results in deletion of the auto-reactive T-cell repertoire during development and peripheral tolerance, which suppresses mature T cells through the development of suppressive T-regulatory cells.

Unique antigens or “neoantigens” result from peptides encoded by a gene, which may be widely expressed throughout normal tissues, but which contains a mutation which is exclusively present in the tumor. The mutation is typically in the coding region of the gene, and some of the mutations may be causal to tumor formation. These very tumor-specific antigens may play an important role in the natural anti-tumor immune response of individual patients, and therefore may be ideally exploited for vaccine or other immunotherapy development (e.g. reviewed in Hacohen et al., Cancer Immunol Res. 2013 July; 1(1): 11-15 and references therein). For example, mice and humans can mount T-cell responses against neoantigens, and mice are tumor protected by immunization with a single mutated peptide. Moreover, memory cytotoxic T lymphocyte (CTL) responses to neoantigens are generated in patients with unexpected long-term survival or those who have undergone effective immunotherapy.

Several different mutation types can lead to the generation of immunotherapeutically useful progenitor sequences comprising or encoding neoepitopes (antigens or immunogens). The first type are somatic point mutations, which lead to the expression of one or more different amino acids in the protein in the tumor. Other mutations lead to the generation of entirely novel tumor-specific protein sequences (progenitor sequences). These include frameshift mutations, which can be either insertions or deletions, and which lead to a new open reading frame with a novel tumor-specific protein sequence. Read-through mutations, in which a stop codon is modified or deleted, allow the translation of a longer protein, and thereby also generate a novel tumor specific progenitor protein sequence. Splice site mutations cause the inclusion of an intron into the mature mRNA and thus a unique tumor-specific progenitor protein sequence. Lastly, chromosomal rearrangements, which lead to the generation of chimeric proteins, create a tumor-specific progenitor sequence at the junction of the two proteins.

In some embodiments, any of the foregoing types of antigens or neoantigen peptides identified on neoORFs and missense neoantigens are synthesized in vitro and linked to conjugates of the present invention. The conjugates and compositions thereof may be administered to a subject with a powerful immune adjuvant and coupled with complementary immunotherapeutics such as checkpoint-blockade inhibitors.

### B. Dendritic Cell Vaccines

Antigen presenting cells (APC), in particular the professional APCs: dendritic cells (DCs) function at the frontier of the immune system and at the interface of the innate and adaptive immune responses, making them uniquely suited for cancer immunotherapy.

In accordance with the present invention, conjugates, and particles and/or formulations packaging conjugates may be used to harness dendritic cells (DCs) to enhance antigen presentations. In some embodiments, conjugates comprising tumor antigens as payloads may be used to prime dendritic cells and primed DCs then can be used as cellular vaccines for treating a cancer.

Approaches that target to dendritic cells directly and indirectly for inducing anti-tumor immune response use a wide variety of ex vivo DC culture conditions, antigen (Ag) source and loading strategies, maturation agents, and routes of vaccination. In general, ex vivo DCs are generated from in vitro differentiation of peripheral blood mononuclear cells (PBMCs) in the presence of stimulating factors including granulocyte-macrophage colony-stimulating factor (GM-CSF) and interleukin (IL)-4 or IL-13 (Alters S E et al., IL-13 can substitute for IL-4 in the generation of dendritic cells for the induction of cytotoxic T lymphocytes and gene therapy, J Immunother., 1999, 22: 229-236).

The DCs can comprise TAA peptides of the invention by any means known or to be determined in the art. Such means include pulsing of dendritic cells with one or more conjugates comprising one or more antigenic peptides. As a non-limiting example, to induce a strong and durable anti-tumor T cell responses, conjugates comprising multiple TAA peptide epitopes may be used to pulse DCs. In general, in vitro cultured autologous DCs are transformed with conjugates comprising one or more TAA peptide epitopes. Pulsed DCs may be amplifies in vitro before infusion.

In accordance with the invention, DC cellular vaccines are dendritic cells that comprise one or more antigenic peptides included in the present compositions.

### C. Adoptive T Cell Immunotherapy (ACT)

Adoptive T cell transfer is a direct strategy to increase the frequency of tumor antigen specific T cells. Tumor antigen specific T cells can be largely expanded in vitro, thus by-pass the early stages of endogenous T cell activation. According to this strategy, conjugates comprising TAA peptide epitopes, alone or in combination with so-stimulatory agents may be coupled to the surface of APCs (e.g., DCs) to activate T cells in vitro.

In some embodiments, conjugates, particles and formulations comprising conjugates may be used to enhance T cells mediated immune response, particularly anti-cancer immune response. The T cells may be from a variety of sources such as a cultured T cell, e.g., a primary T cell, or a T cell from a cultured T cell line, e.g., Jurkat, SupT1, etc., or a T cell obtained from a subject. If obtained from a subject, the T cell can be obtained from numerous sources, including but not limited to blood, bone marrow, lymph node, the thymus, or other tissues (e.g., tumor tissue) or fluids. T cells can also be enriched for or purified. In some aspects, T cell is a human T cell. The T cell can be any type of T lymphocyte and can be of any developmental stage, including but not limited to, CD4⁺/CD8⁺ double positive T lymphocytes, CD4⁺ helper T lymphocytes, e.g., Th₁ and Th₂ cells, CD8⁺ T lymphocytes (e.g., cytotoxic T lymphocytes), peripheral blood mononuclear cells (PBMCs), peripheral blood leukocytes (PBLs), tumor infiltrating lymphocytes (TILs), memory T cells, naive T cells, and the like.

TIL (tumor infiltrating lymphocytes) expansion: In some embodiments, tumor infiltrating lymphocytes (TILs) which are a class of lymphocytes derived from primary or metastatic tumor tissue fragments, regionally tumor-draining lymph nodes or malignant ascites, may be expanded in vitro in IL-2-supplemented media and enriched predominantly in CD8+ cytotoxic T lymphocytes (CTLs) in order to eradicate autologous tumor antigens in a MHC-restricted pattern. In this strategy, conjugates comprising co-stimulatory molecules, and/or cytokines that can support T cell growth and maintenance, may be used to expand in vitro TLRs. In that strategy, a patient, prior to T cell infusion, is conditioned with a lymphodepleting regimen, and then is given IL-2 post infusion.

CD4+ or CD8+ antigen specific T cell clones: Some studies have proven that neoantigens of mutated peptides identified in a cancer subject may be the major natural targets of tumor specific TILs (Lenneiz et al., The response of autologous T cells to a human melanoma is dominated by mutated neoantigens. Proc Natl Acad Sci USA, 2005, 102: 16013-16018). Accordingly, conjugates comprising tumor specific -neoantigens may be used ex vivo for expanding patient derived T cells such as PBMCs before adoptive T cell therapy.

In this context, conjugates comprising tumor specific neoantigens may further comprises one or more non-specific T cell receptor stimulating agent as payloads, wherein the non-specific T cell receptors stimulators may be a T cell growth factor, including but not limited to interleukin (IL)-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2. IL-12 is a preferred T-cell growth factor. The T cell growth factor may be included in the same conjugate as one or more tumor specific antigens, or the T cell growth factor may be in separate conjugate but is packaged together with the conjugates comprising one or more tumor specific antigens in the same particle or other formulations.

In this strategy, The method for producing activated cytotoxic T lymphocytes (CTL), comprises contacting in vitro autologous T cells from a patient himself/herself with TAA antigenic peptide loaded class I MHC/HLA molecules expressed on the surface of a suitable APC (e.g., a DC) or an artificial composition mimicking an antigen-presenting cell for a period of time sufficient to activate said CTL in an antigen specific manner.

Engineered T cells: Engineered autologous T cells may be used for adoptive T cell immunotherapy. Autologous T cells may be engineered to express a defined T cell receptor (TCR) that are directed against target TAAs, either wild-type TCR, or mutated/engineered TCR towards a higher affinity to the antigen peptide/MHC molecule complexes. Alternatively, a genetic engineered novel receptor consisting of a chimera between an antibody molecule and TCR segments (Chimeric Antigen Receptor, CAR) may be used for transduction of autologous T cells.

CAR-engineered T cells combine TAA-recognized single-chain antibody with the activation motif of T cells, freeing antigen recognition from MHC restriction and thus breaking one of the barriers to more widespread application of ACI. It means combining the high affinity of antibody to TAA with the killing mechanism of T cells. It had been bolstered that CAR-engineered T cells exhibited antitumor function to prostate cancer and other advanced malignancies. As non-limiting examples, CARs may include NKG2D based CARs (Sentman and Meetah, NKG2D CARs as cell therapy for cancer, 2014, Cancer J., 20(2): 156-159); CD28z CARs and armored CARs (reviewed by Pegram et al., CD28z CARs and armored CARs, 2014, Cancer 20(2): 127-133).

In some aspects, conjugates comprising active agents that can promote T cell migration and function may be transduced into engineered T cells to facilitate, after T cell infusion, the trafficking of infused T cells to tumor sites and penetrating the tumor microenvironments and functional maintenance.

D. γδ T cells

γδ T cells are a special type of T lymphocytes which were found to act as interface for the cross talk between innate and cell-mediated immune cells, because of its expression of both natural killer receptors and γδ T cell receptors (Wu Y L, et al. γδ T cells and their potential for immunotherapy. Int J Biol Sci 2014; 10: 119-35). γδ TCR recognize non-peptide antigens like glycerolipids and other small molecules, polypeptides that are soluble or membrane anchored, and/or cross linked to major histocompatibility complex (MHC) molecules or MHC-like molecules in an antigen-independent manner (Reviewed by Born et al., Diversity of gammadelta T-cell antigens. Cell Mol Immunol, 2013, 10(1):13-20). γδ T cells have a unique role in the immune-surveillance against malignancies as they can directly recognize molecules that are expressed on cancer cells without need of antigen processing and presentation.

γδ T cells may be used to cross-present antigens to effector T cells with αβ receptor as effective APCs (See, e.g., U.S. Pat. No. 8,338,173). In some embodiments, γδ T cells may be isolated and enriched in vitro from human peripheral blood cells. Isolated and expanded γδ T cells may be stimulated or loaded with tumor antigens comprised in conjugates, particles and formulations as discussed in the present application. Prior to loading tumor antigens, in vitro isolated γδ T cells may be stimulated using (E)-4-hydroxy-3-methyl-but-2-enyl pyrophosphate (HMBPP), isopentenyl pyrophosphate (IPP) or other small molecular weight non-peptide compounds with selectivity for γδ T cells, or other stimulators for induction of antigen-uptake, of presentation function and of expression of co-stimulatory molecules, e.g., phytohemagglutinin (PHA). Stimulated γδ T cells may be loaded with one or more conjugates comprising one or more TAA peptide epitopes. The loaded γδ T cells may be used to activate anti-tumor T cells as antigen presenting cells (APCs). TAA-loaded γδ T cells may be used to prime naïve T cells to generate effector T cells.

### E. Manipulation of Costimulatory Pathways

Given the critical role of costimulatory receptors in regulating T cell activation, pharmaceutical manipulation of these pathways can be a promising therapeutic approach. In accordance with the present invention, compositions may be used as agonistic agents to ligate the positive costimulatory receptors, or blocking agents that attenuate signaling through inhibitory receptors. In one example, a conjugate that comprises an antibody against the positive costimulatory receptor 4-1BB (CD137), or an antibody against OX40, or antibodies against CD137 and OX40, may be used as an agonistic agent. In another example, a conjugate that comprises an antibody against the inhibitory receptor cytotoxic T-lymphocyte antigen-4 (CTLA-4), or a fragment thereof, may be used as a blocking agent to inhibit the immunosuppression.

In some embodiments, agonistic agents and blocking agents of the present conjugates may be used in combination with other immunological conjugates, in particular, conjugates comprising active agents (e.g. TAAs, and antigenic peptides) that can stimulate initial antigen recognition of TCR.

### F. Cytokine Based Immunotherapy

Compositions, such as conjugates, particles and/or formulations of the present invention may be used for cytokine based immunotherapy.

In some embodiments, immunological conjugates of cytokines may be used to expand cytotoxic T cells. If a lower level of endogenous T cell priming has occurred in a tumor patient, cytokines, such as T cell growth factor, may be used to expand these activated T cells. In this strategy, conjugates comprising such cytokines, or particles/formulations that comprise such conjugates may be administered to the patient to expand activated T cells in vivo. For example, conjugates comprising IL-2, IL-7, IL-12 or in combination thereof, as a payload may be used for this purpose.

Conjugates comprising cytokines may also be used to induce killer cells (known as cytokine induced killer cells CIK) are a heterogeneous population of effector CD8+T cells with diverse TCR specificities, possessing non-MHC restricted cytolytic activities against tumor cells with the dual characteristics of T cells and NK cells, which could identify the target cells not only through the TCR and MHC, but also could through the Natural Killer (NK) cell activated receptor.

### G. Antibody Based Immunotherapy

Conjugates and compositions of the present invention comprising antibodies or fragments thereof against a tumor specific antigen may be used for treatment of cancer. Monoclonal antibodies that elicit an antigen-antibody response specific to tumor specific antigens (TAAs) induce various types of immune response including cell-mediated cytotoxicity (ADCC) and complement-dependent cytotoxicity (CDC), etc. to attack cancer cells, thereby inducing cell death. Antibodies against TAAs as target are disclosed in the art and can be incorporated to the conjugates or particles of the present invention, such as antibodies against 17-1A (also known as EpCAM, EGP-40 or GA 733-2 (U.S. Pat. No. 7,632,925); oculospanin (U.S. Pat No. 7,361,340); antibodies in U.S. Pat. Nos. 8,637,084; 8,444,974; 8,034,902; 7,785,816; 7,824,678; 7,626,011; 7,691,372; 7,674,883; 7,378,091; 7,288,248; 7,232,888; 5,824,311; 5,876,691; 5,688,657; 5, 639,622; 5,637,493; 4,960,716; and US patent publication No.: 2011/0135570; and Chimeric antibodies disclosed in U.S. Pat. No. 5,354,847; the contents of which are incorporated by reference in their entirety.

H. Allogeneic Stem Cell Transplantation (alloSCT)

AlloSCT from a compatible donor peripheral blood has gained recognition as a potential immunotherapy for a number of different hematological malignancies and in advanced solid malignant tumors such as mRCC and castration resistant prostate cancer (CRPC). Immunological conjugates of the present invention may be used to prime stem cells for transplantation.

### I. Innate Immune Response

In some embodiments, conjugates and other compositions of the present invention may be used to enhance an innate immune response to increase the anti-cancer immunity in a subject.

### J. Targeting Immunologic Barriers in the Tumor Microenvironment

In addition to elicit a positive cancer specific immune response, strategies may also aim to break the major barriers to immune-mediated tumor destruction. In these strategies, conjugates of the present invention are used to block or reverse inhibitory mechanisms in tumors. In one example, conjugates that comprise antibodies against PD-1, or antibodies against PD-L1, or both may be used to block the interaction between PD-1/PD-L1. Another inhibitory receptor may be LAG-3 which is expressed on activated T cells.

It has been shown that some tumors show increased expression of the immunosuppressive enzyme indoleamine-2,3-dioxygenase (IDO), which is a metabolic enzyme that metabolizes tryptophan and limits T- and NK-cell activation in local tissue microenvironments. Blockade of IDO activity can be immune-potentiating in some tumors. In this strategy, conjugates comprising one or more small molecule IDO inhibitor may be used to block its activity.

In some embodiments, conjugates that comprise active agents which can deplete Treg cells, or myeloid-derived suppressor cells (MDSCs) in the tumor microenvironment. Such active agents may be antibodies against components of Treg cells MDSCs, for example, antiCD25 antibody.

### K. Combination Immunotherapy

In some embodiments, an effective immunotherapy may combine different interventions including strategies to increase systemically the frequency of anti-cancer T cells, strategies to overcome distinct immune suppressive pathways within the tumor microenvironment and strategies to trigger innate immune activation and inflammation in tumor sites.

### V. Applications

### A. Cancer

In accordance with the present invention, conjugates, particles and formulations comprising conjugates and vaccines may be used to treat cancer; the cancer may be any cancer, including but not limited to any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the vulva, chronic lymphocytic leukemia, chronic myeloid cancer, cervical cancer, glioma, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, peritoneum, omentum, and mesentery cancer, pharynx cancer, prostate cancer, rectal cancer, renal cancer, skin cancer, soft tissue cancer, testicular cancer, thyroid cancer, ureter cancer, urinary bladder cancer, and digestive tract cancer such as, e.g., esophageal cancer, gastric cancer, pancreatic cancer, stomach cancer, small intestine cancer, gastrointestinal carcinoid tumor, cancer of the oral cavity, colon cancer, and hepatobiliary cancer.

### Equivalents and Scope

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

### EXAMPLES

### Example 1

### Preparation of Vaccine Conjugates

An antigen or tumor antigen is prepared as a component of a conjugate. In some embodiments, the antigen or tumor antigen is a shared antigen or neoantigen. The binding of conjugate moiety to antigen presenting cells is measured by flow cytometric analysis and/or fluorescence-activated cell sorting (FACS).

Once the antigen presenting cells have internalizled the conjugate, presentation occurs via the MHC presentation system of the cells.

### T Cell Lines

Antigen containing conjugate specific T cell lines are generated according to published methodologies.

### Immune Assays

Antigen containing conjugate stimulated PBMCs are cultured with T cells and evaluated using the ELISPOT assay (MBL, Nagoya, Japan) in 96-well ELISPOT plate (MultiScreen HTS, Millipore) and counted by an ELISPOT reader (CTL Technologies).

### Cytotoxicity

Antigen containing conjugate stimulated PBMCs are also tested for cytotoxicity against one or more cancer cells.

Presentation on the surface of APCs then triggers the immune response of T-cells and other immune cells in response to the presentation of the antigen of the conjugate.

What is claimed is:

1. A conjugate for eliciting a cancer specific immune response comprising the structure of the formula X—Y—Z, wherein X is a targeting moiety; Y is a linker; and Z is an active agent that is capable of increasing a cancer specific immune response.

2. The conjugate of claim 1, wherein the active agent Z is a tumor specific antigenic peptide, wherein said antigenic peptide is derived from a tumor specific antigen (TAA) selected from the group consisting of an oncofetal antigen, an oncoviral antigen, an overexpressed tumor antigen, a cancer-testis antigen, a neoantigen, and a post-translationally modified antigen.

3. The conjugate of claim 2, wherein the active agent is a MHC/HLA class I specific antigenic peptide or a MHC/HLA class II specific antigenic peptide.

4. The conjugate of claim 3, wherein the peptide is about 6 amino acids to about 30 amino acids in length.

5.-6. (canceled)

7. The conjugate of claim 2, wherein the active agent is a B cell specific antigenic peptide.

8. The conjugate of claim 2, wherein the antigenic peptide is a naturally occurred peptide or an analog thereof.

9. (canceled)

10. The conjugate of claim 2 comprising two or more antigenic peptides.

11.-13. (canceled)

14. The conjugate of claim 10, wherein said two or more antigenic peptides are a mixture of MHC/HLA class I specific antigenic peptides and MHC/HLA class II specific antigenic peptides.

15. The conjugate of claim 2, wherein the targeting moiety is a tumor specific antigenic peptide, which is derived from a tumor specific antigen (TAA) selected from the group consisting of an oncofetal antigen, an oncoviral antigen, an overexpressed tumor antigen, a cancer-testis antigen, a neoantigen, and a post-translationally modified antigen.

16.-17. (canceled)

18. The conjugate of claim 1, wherein the active agent is an agent that can stimulate the proliferation, maturation, migration and antigen presentation of dendritic cells.

19. The conjugate of claim 18, wherein the active agent is a chemokine selected from CCL3 (MIP1α), CCL5 (RANTES), CCL7 (MCP-3), CCL8 (MCP-2), CCL9 (MRP-2), CCL14 (HCC1), CCL16 (HCC4); CCL2 (MCP-1), CCL7 (MCP-3), CCL12 (MCP-5), CCL8 (MCP-2), CCL16 (HCC4); CCL17 (TARC), CCL19 (MIP-3β, ELC), CCL3 (MIP1α), CCL4 (MIP1β), CCL5 (RANTES), CCL8 (MCP-2), CCL11 (eotaxin), CCL14 (HCC1), CCL16 (HCC4), CCL20 (MIP-3α), CCL1 (TCA3), CCL25 (TECK), CXCL9 (Mig), CXCL10 (IP10), CXCL11 (ITAC), CX3Cl1 (fractalkine), CCL12 (SDF-1), CCL19 (MIP-3β, ELC), and CCL21 (6-Ckine, SLC).

20. The conjugate of claim 1, wherein the active agent is an agent that can stimulate the proliferation, recruitment and activation of a cancer specific T cells.

21. The conjugate of claim 20, wherein T cells is CD8+ T cell and CD4+ T cells

22. The conjugate of claim 21, wherein the active agent is an agonistic agent of a co-stimulatory molecule.

23. The conjugate of claim 22, wherein the co-stimulatory molecule includes CD7, B7-1 (CD80), B7-2 (CD86), 4-1BBL receptor (CD137), 4-1BB ligand (CD137-L), OX40L, inducible co-stimulatory ligand (ICOS-L), intercellular adhesion molecule (ICAM), CD2, CD5, CD9, CD30L, CD40, CD70, CD83, HLA-G, MICA, MICB, HVEM, lymphotoxin beta receptor, 3/TR6, ILT3, ILT4, HVEM, GITR, GITR-L, TLR agonist, B7-H3 ligand, CD27, CD28, 4-IBB, OX40, CD30, CD40, CD226, ICOS, LFA-1, CD2, CD7, LIGHT, NKG2C, and B7-H3.

24. The conjugate of claim 21, wherein the active agent is a T cell adhesion molecule which is selected from CD11a (LFA-1), CD11c, CD49d/29(VLA-4), CD50 (ICAM-2), CD54 (ICAM-1), CD58 (LFA-3), CD102 (ICAM-3), CD106 (VCAM), and antibodies to selectins L, E, and P.

25. The conjugate of claim 21, wherein the active agent is a cytokine selected granulocyte macrophage colony stimulating factor (GM-CSF), tumor necrosis factor alpha (TNFα), tumor necrosis factor beta (TNFβ), macrophage colony stimulating factor (M-CSF), interleukin-1 (IL-1), interleukin-2 (IL-2), interleukin-4 (IL-4), interleukin-5 (IL-5), interleukin-6 (IL-6), interleukin-10 (IL-10), interleukin-12 (IL-12), interleukin-15 (IL-15), interleukin-21 (IL-21), interferon alpha (IFNα), interferon beta (IFNβ), interferon gamma (IFNγ), and interferon-gamma inducing factor (IGIF).

26. The conjugate of claim 21, wherein the active agent may be a TCR, engineered TCR, a chimeric antigen receptor (CAR), or a T cell coreceptor.

27. The conjugate of claim 26, wherein the TCR is specific to a tumor associated antigen.

28. The conjugate of claim 1, wherein the active agent may be an antibody specific to a tumor antigen, a TLR agonist, a chemokine, a cytokine, or a cytotoxic agent.

29. The conjugate of claim 1, wherein the active agent is a CD3 antibody or a CD3-binding fragment thereof, or a CD16 antibody or a CD16-binding fragment thereof.

30. (canceled)

31. The conjugate of claim 1, wherein the active agent is a cell surface antigen or a fragment thereof.

32.-33. (canceled)

34. The conjugate of claim 1, wherein the active agent is mifamurtide.

35. The conjugate of claim 1, wherein the linker is a cleavable linker.

36.-37. (canceled)

38. The conjugate of claim 1, wherein the linker is selected from the group consisting of an alkyl chain, a peptide, a beta-glucuronide, a self-stabilizing group, a hydrophilic group and a disulfide group.

39. (canceled)

40. The conjugate of claim 1, wherein the targeting moiety is an antibody, an antibody fragment, scFv, or an antibody mimic, which specifically binds to a tumor cell, a tumor antigen, or tumor infiltrating immune cells.

41. The conjugate of claim 40, wherein the targeting moiety specifically targets to tumor infiltrating T lymphocytes (CTLs) or dendritic cells.

42. (canceled)

43. The conjugate of claim 1, wherein the targeting moiety is an aptamer.

44. The conjugate of claim 1, wherein the targeting moiety X is a targeting moiety complex comprising a target binding moiety (TBM) and a masking moiety (MM) attached to the TBM via a cleavable moiety (CM).

45. The conjugate of claim 44, wherein the MM is a peptide.

46. The conjugate of claim 44, wherein the CM is cleaved by an enzyme.

47. The conjugate of claim 46, wherein the enzyme is selected from the group consisting of MMP1, MMP2, MMP3, MMP8, MMP9, MMP14, plasmin, PSA, PSMA, CATHEPSIN D, CATHEPSIN K, CATHEPSIN S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, and TACE.

48. The conjugate of claim 44, wherein the CM is cleaved by a reducing agent.

49. The conjugate of claim 48, wherein the CM comprises a disulfide bond.

50. The conjugate of claim 44, wherein the binding of the TBM to its target is inhibited or hindered sterically with the presence of MM.

51. The conjugate of claim 1, wherein the targeting moiety X is a targeting moiety complex comprising a target binding moiety (TBM) attached to a photocleavable moiety.

52. (canceled)

53. The conjugate of claim 51, wherein the photocleavable moiety is selected from nitorphenyl methyl alcohol, 1-nitrophenylethan-1-ol and substituted analogues.

54. The conjugate of claim 53, wherein the photocleavable moiety couples to hydroxy or amino residues present in the TBM.

55. (canceled)

56. The conjugate of claim 1, further comprising a reacting group that reacts with a functional group on a protein or an engineered protein or derivatives/analogs/mimics thereof.

57. The conjugate of claim 56, wherein the protein is a naturally occurring protein such as a serum or plasma protein, or a fragment thereof.

58. The conjugate of claim 57, wherein the protein is thyroxine-binding protein, transthyretin, α1-acid glycoprotein (AAG), transferrin, fibrinogen, albumin, an immunoglobulin, α-2-macroglobulin, a lipoprotein, or a fragment thereof.

59. The conjugate of claim 1, further comprising a pharmacokinetic modulating unit.

60. The conjugate of claim 59, wherein the pharmacokinetic modulating unit is a natural or synthetic protein or fragment thereof, a natural or synthetic polymer, or a particle.

61. The conjugate of claim 60, wherein the pharmacokinetic modulating unit comprises a polysialic acid unit, a hydroxyethyl starch (HES) unit, or a polyethylene glycol (PEG) unit.

62. The conjugate of claim 60, wherein the pharmacokinetic modulating unit comprises dendrimers, inorganic nanoparticles, organic nanoparticles, or liposomes.

63. A nanoparticle for eliciting a cancer specific immune response comprising the conjugate of claim 1.

64. The nanoparticle of claim 63, wherein the nanoparticle comprises a polymeric matrix.

65. The nanoparticle of claim 64, wherein the polymeric matrix comprises one or more polymers selected from the group consisting of hydrophobic polymers, hydrophilic polymers, and copolymers thereof.

66.-67. (canceled)

68. The nanoparticle of claim 64, wherein the polymeric matrix comprises one or more polymers selected from the group consisting of poly(lactic acid), poly(glycolic acid), poly(lactic-co-glycolic acid), poly(ethylene oxide), poly(ethylene glycol), poly(propylene glycol), and copolymers thereof.

69. The nanoparticle of claim 64, wherein the size of the nanoparticle is between 10 nm and 5000 nm.

70. (canceled)

71. The nanoparticle of claim 63, wherein the weight percentage of the conjugate is between 0.1% and 35%.

72. A pharmaceutical formulation for eliciting a cancer specific immune response comprising the conjugate of claim 1.

73. The pharmaceutical formulation of claim 72, wherein the formulation is a cancer vaccine which further comprising one or more excipient.

74. The pharmaceutical formulation of claim 73 further comprising at least one adjuvant.

75. A method for priming an immune cell comprising contacting the conjugate of claim 1 with an immune cell.

76. The method of claim 75, wherein the active agent of the conjugate comprises one or more tumor specific antigenic peptides; wherein the immune cell is an antigen presenting cell.

77. The method of claim 76, wherein the antigen presenting cell is a dendritic cell.

78.-89. (canceled)

90. A method for treating a cancer in a subject comprising administering to the subject a pharmaceutically effective amount of the conjugate of claim 1.
